(19) United States
(12) Patent Application Publication
(10) Pub. No.: US 2026/0108475 A1
(43) Pub. Date: Apr. 23, 2026

(54) PROCESS FOR INSERTING TARGETING LIGAND INTO A LIPID NANOPARTICLE ENCAPSULATING NUCLEIC ACID AND COMPOSITIONS PRODUCED THEREFROM

(71) Applicant: Genzyme Corporation, Cambridge, MA (US)

(72) Inventors: Amey Bandekar, Acton, MA (US); Isidro Zarraga, Waltham, MA (US)

(21) Appl. No.: 19/364,189

(22) Filed: Oct. 21, 2025

Related U.S. Application Data
(60) Provisional application No. 63/710,457, filed on Oct. 22, 2024.

(51) Int. Cl. A61K 9/51; A61K 31/7105; A61K 47/18; A61K 47/22; A61K 47/68

(52) U.S. Cl. CPC A61K 9/5192; A61K 9/5123; A61K 31/7105; A61K 47/18; A61K 47/22; A61K 47/6835

(57) ABSTRACT

Methods for inserting a targeting moiety (such as an antigen-binding protein moiety, a fragment antigen-binding moiety, or the like) into a lipid nanoparticle and compositions resulting from such methods are provided. The methods generally utilize a reaction that forms targeted nanoparticles by combining a targeting moiety and a lipid nanoparticle. A quenching operation is then provided by cooling the reaction to a temperature that stops the insertion of the targeting moiety. The reaction substantially preserves the integrity of a nucleic acid cargo (such as an mRNA encoding a VHH binding molecule).

### CROSS REFERENCE TO RELATED APPLICATIONS

The present application claims priority U.S. Provisional Patent Application No. 63/710,457, filed Oct. 22, 2024, each of which is hereby incorporated by reference herein in its entirety for all purposes.

### TECHNICAL FIELD

Lipid nanoparticles (LNPs) are nanostructures that have the ability to encapsulate nucleic acids and deliver them into cells of subject in a non-specific manner. Targeted delivery is a precise and effective strategy in medicine for targeted delivery of a therapeutic to a target destination in a subject's body. Targeted delivery enables, e.g., accurate drug delivery to tumor cells or tissues, enhancing therapeutic effects while minimizing undesirable side effects on normal cells or tissues. The present disclosure relates to processes for insertion of a targeting moiety(ies) into a lipid nanoparticle, as well as the products of the process.

### SUMMARY

Unless otherwise defined, all technical and scientific terms used herein have the same meaning as commonly understood by one of ordinary skills in the art to which this invention belongs. Methods (also referred to herein as “processes”) and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

In some aspects, the disclosure provides a process for the insertion of a targeting moiety into a lipid nanoparticle, the process comprising the steps of: inserting the targeting moiety into the lipid nanoparticle in a reaction that combines an amount of a targeting moiety with an amount of total lipids in a lipid nanoparticle for a time-period of at least one hour at a temperature no higher than 42° C., thereby forming an amount of targeted lipid nanoparticles; and quenching the insertion of the targeting moiety by cooling the reaction to about 20° C. or lower.

In some aspects, the disclosure provides a process for the insertion of a targeting moiety into a lipid nanoparticle, the process comprising the steps of inserting the targeting moiety into the lipid nanoparticle in a reaction that combines at least 0.15 μg of the targeting moiety per gram of the total lipids in the lipid nanoparticle for a time-period of at least one hour at a temperature that does not exceed 54° C., thereby forming an amount of targeted nanoparticles; quenching the insertion of the targeting moiety by cooling the reaction to about 20° C. or lower.

In some aspects, the disclosure provides a process for the insertion of a targeting moiety into a lipid nanoparticle, the process comprising the steps of: inserting from 0.15 μg to 30 g of the targeting moiety into the lipid nanoparticle in a soluble reaction by combining from 0.15 μg of the targeting moiety per gram of the total lipid nanoparticle to 30 g of the targeting moiety per gram of the total lipid nanoparticle for a time-period of at least one hour at a temperature that does not exceed 54° C., whereby the soluble reaction produces a targeted nanoparticle comprising from 0.15 μg to 30 mg of the targeted moiety incorporated into its outer membrane.

In some embodiments that can be combined with any of the above embodiments, the process produces a targeted nanoparticle comprising from 0.15 μg to 25 mg, from 0.15 μg to 20 mg, from 0.15 μg to 15 mg, from 0.15 μg to 10 mg, from 0.15 μg to 5 mg, from 0.15 μg to 1 mg of the targeted moiety incorporated into its outer membrane. In some embodiments that can be combined with any of the above embodiments, the process yields greater than 80% of targeted nanoparticles. In some embodiments that can be combined with any of the above embodiments, the inserting is conducted at a pH from about 6.2 to about 7.5. In some embodiments that can be combined with any of the above embodiments, the time-period is a time-period from about one hour to about four hours, or from about one hour to about two hours, or the time-period is no more than four hours at a temperature between 20° C. and 42° C. In some embodiments that can be combined with any of the above embodiments, the insertion is performed on a 2-(N-morpholino)ethanesulfonic acid (MES) buffer or a tris(hydroxymethyl)aminomethane (Tris) or a HEPES (4-(2-hydroxyethyl)-1-piperazineethanesulfonic acid) (HEPES) buffer, for example the buffer can be a 25 mM MES buffer comprising 150 mM NaCl at pH 6.5, 50 mM Tris buffer comprising 150 mM NaCl at pH 7.5, or 25 mM HEPES buffer comprising 150 mM NaCl at pH 7.4. In some embodiments that can be combined with any of the above embodiments, the targeting moiety is from about 10 kDA to about 160 kDA. In some embodiments that can be combined with any of the above embodiments, the targeting moiety is an antigen-binding protein, e.g., a fragment antigen-binding (Fab) or a variable heavy domain (VHH). In some embodiments that can be combined with any of the above embodiments, the targeting moiety is encapsulated in a polymeric micelle, e.g, a polyethylene glycol-micelle (PEG-micelle). In some embodiments that can be combined with any of the above embodiments, the lipid nanoparticle comprises at least one ionizable lipid, e.g.,

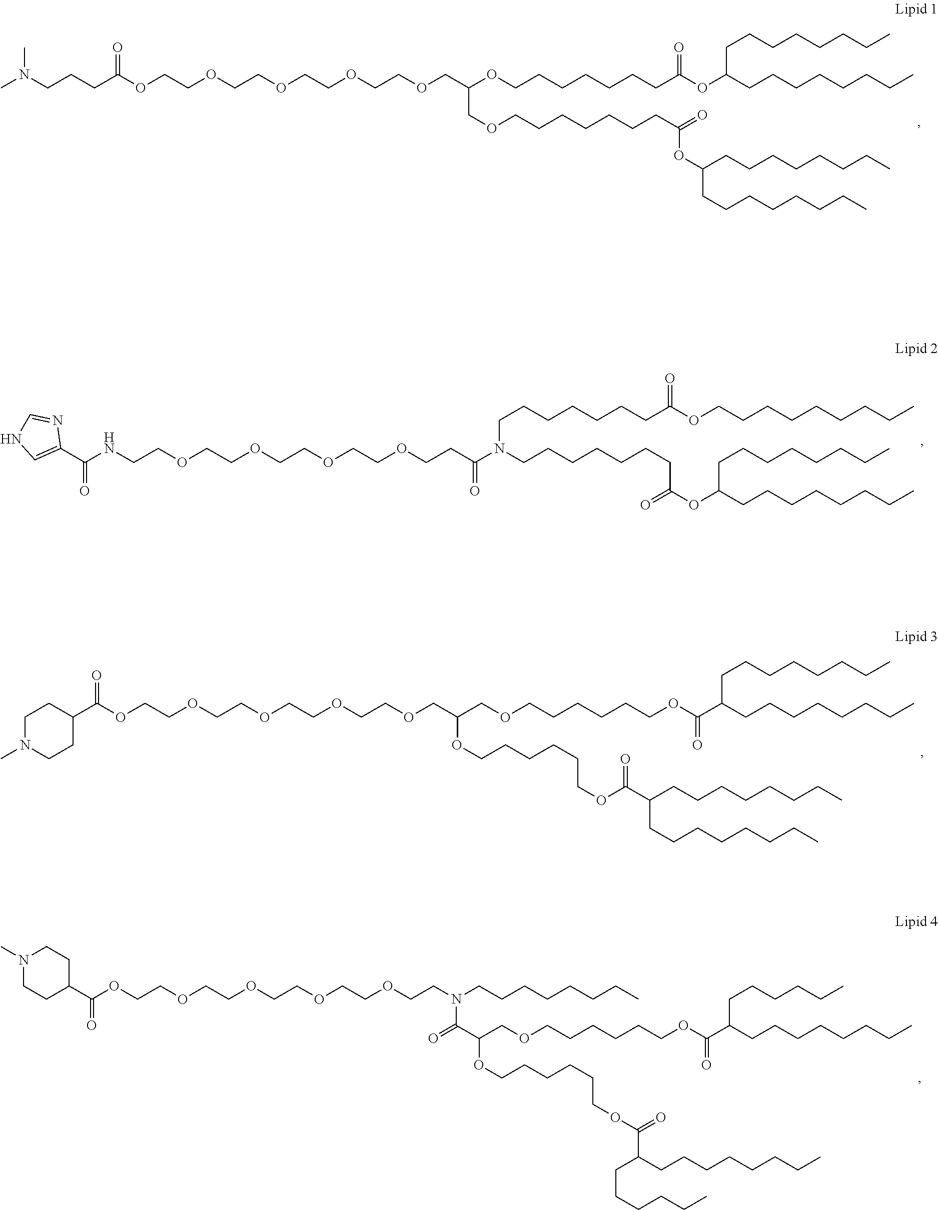

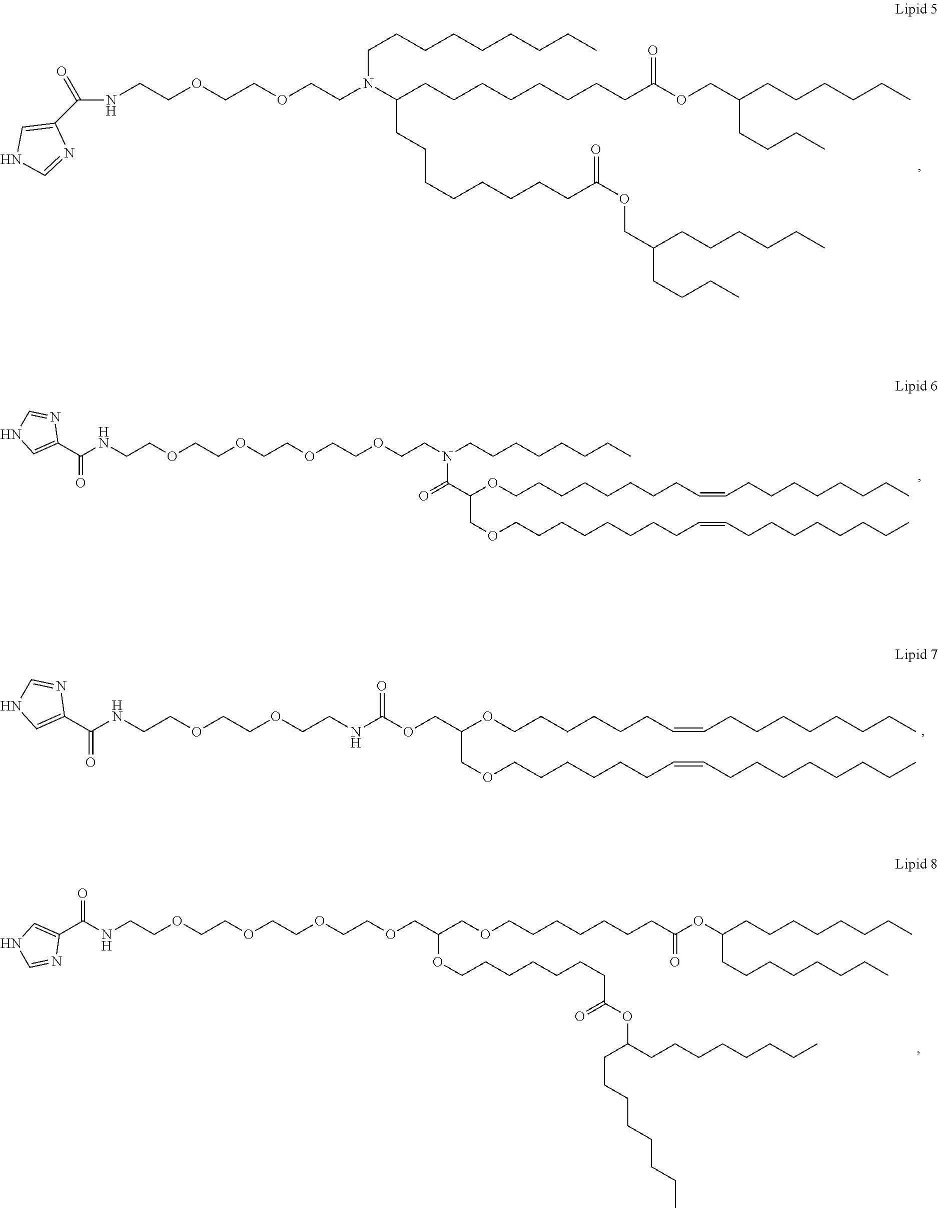 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC ; CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1 ; CCCCCCCC/C=C\CCCCCCOCC(COC(=O)NCCOCCOCCNC(=O)c1c[nH]cn1)OCCCCCC/C=C\CCCCCCCC ; CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

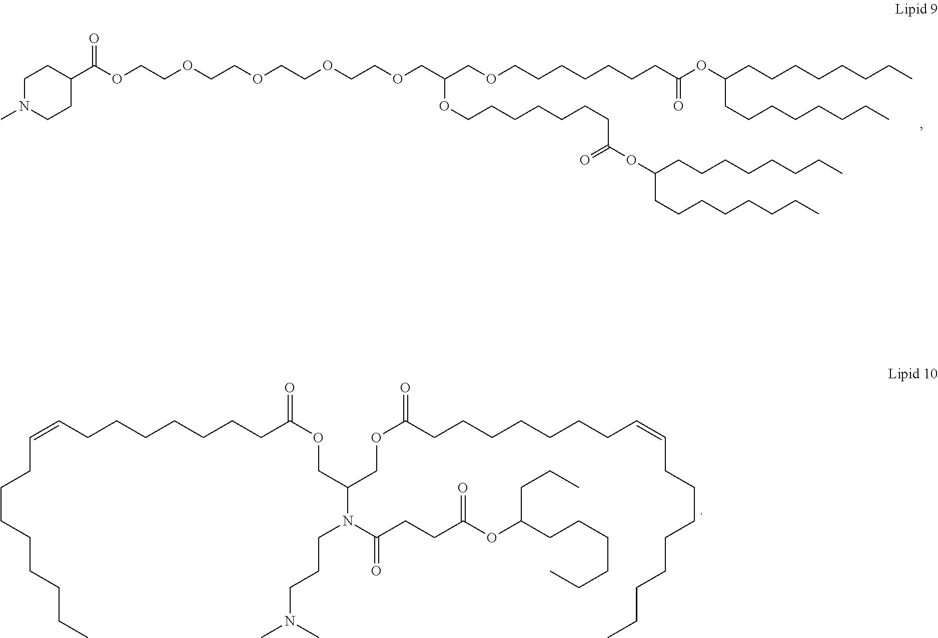 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC ; CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In some embodiments that can be combined with any of the above embodiments, the inserting occurs at a rotational speed of zero revolutions per minute (0 RMPs) during the time-period (i.e. no shaking is required). In other embodiments, the rotational speed is no more than three hundred revolutions per minute (300 RMPs) or no more than six hundred revolutions per minute (600 RMPs) during the time-period. In some embodiments that can be combined with any of the above embodiments, the lipid nanoparticle further comprises a nucleic acid cargo, e.g., an mRNA encoding a VHH antigen binding moiety. In some embodiments that can be combined with any of the above embodiments, the nucleic acid cargo is pre-encapsulated into the lipid nanoparticle prior to the insertion of the targeting moiety. In some embodiments that can be combined with any of the above embodiments, insertion of the targeting moiety does not fragment a nucleic acid cargo is pre-encapsulated into the lipid nanoparticle prior to the insertion of the targeting moiety. In some embodiments that can be combined with any of the above embodiments, the targeted lipid nanoparticle remains substantially unfragmented during at least 3 freeze thaw cycles. In some embodiments that can be combined with any of the above embodiments, greater than 50% of the nucleic acid cargo remains encapsulated in the targeted lipid nanoparticle during the reaction. In some embodiments that can be combined with any of the above embodiments, the lipid nanoparticle ranges from approximately 50 nanometers to 200 nanometers in size, from approximately 75 nanometers to 85 nanometers in size, from approximately 85 nanometers to 97 nanometers in size, or from approximately 80 nanometers to 90 nanometers in size. In some embodiments that can be combined with any of the above embodiments, the insertion of the targeting moiety into the lipid nanoparticle increases the size of the lipid nanoparticle between 0% to 25%. In some embodiments that can be combined with any of the above embodiments, the insertion of the targeting moiety into the lipid nanoparticle increases the size of the lipid nanoparticle by about 20%. In some embodiments that can be combined with any of the above embodiments, the targeted nanoparticle is stable for a time-period of at least 1 day at a temperature between 2° C. and 8° C. In some embodiments that can be combined with any of the above embodiments, the lipid nanoparticle comprises an amount of an ionizable lipid, an amount of cholesterol, an amount of neutral lipid, and an amount of a PEG-lipid, e.g., in some instances the lipid nanoparticle comprises amounts of the ionizable lipid, the cholesterol, the neutral lipid, and the PEG-lipid at molar ratio ranges respectively of 40%-60% ionizable lipid, 20%-35% cholesterol, 10%-30% of neutral lipid, 0.5%-5% of PEG-lipid. In some instances the ionizable lipid is selected from the group consisting of a Lipid 1, Lipid 2, Lipid 3, Lipid 4, Lipid 5, Lipid 6, Lipid 7, Lipid 8, Lipid 9, Lipid 10, a KC2 lipid, a DLin-MC2-DMA lipid, a DLin-MC3-DMA lipid, a DSDMA lipid, a DODMA lipid, a DLinDMA lipid, a DLenDMA lipid, a γ-DLenDMA lipid, a DLin-K-DMA lipid, a DLin-C2K-DMA lipid, a DLin-K-C3-DMA lipid, a DLin-K-C4-DMA lipid, a DLen-C2K-DMA lipid, a γ-DLen-C2K-DMA lipid, or a DLin-MP-DMA lipid. In some instances, the neutral lipid is a phosphatidylcholine lipid or a phosphatidylethanolamine lipid. In some instances, the phosphatidylcholine lipid or the phosphatidylethanolamine lipid is selected from the group consisting of DOPE, DSPC, DPPC, and POPC.

In some embodiments, the disclosure provides a targeted nanoparticle produced with methods disclosed herein. In some instances, the disclosure provides a targeted lipid nanoparticle produced by a process comprising the steps of: combining in a soluble reaction from 0.15 μg of the targeting moiety per gram of the total lipid nanoparticle to 30 g of the targeting moiety per gram of the total lipid nanoparticle for a time-period of at least one hour at in a lipid nanoparticle having a nucleic acid cargo encapsulated therein, whereby the soluble reaction is conducted for a time-period of at least one hour at a temperature no higher than 42° C., thereby producing an amount of targeted lipid nanoparticles whereby the nucleic acid cargo within the targeted lipid nanoparticles is less than 20% degraded compared to a nucleic acid cargo in a particle where the targeting moiety is not inserted by the process.

These aspects and other features and advantages of the invention are described below in more detail.

### BRIEF DESCRIPTION OF THE FIGURES

The foregoing and other features and advantages of the present invention will be more fully understood from the following detailed description of illustrative embodiments taken in conjunction with the accompanying drawings in which:

FIG. 1 is a schematic of steps in a process for insertion of a targeting moiety into a lipid nanoparticle.

FIG. 2 is a schematic of a composition comprising a targeted lipid nanoparticle.

FIG. 3 is a schematic of various process development parameters for inserting a targeting moiety into a lipid nanoparticle in a reaction. The schematics illustrate a pre-formed LNP with a nucleic acid cargo (e.g., mRNA) as an input to the reaction. The schematic depicts an antigen-binding protein complexed as a micelle as a second input to the reaction. The schematic shows combinations of various insertion parameters considered, including, temperatures (ranging from 20° C. to 54° C.), incubation time (ranging from 30 min to 8 hours), mixing (ranging from 0 RPMs to 600 RPMs). The schematic illustrates that the reaction mix can then be subject to a cooling step, which quenches the reaction inserting the antigen binding protein micelle into the lipid nanoparticle. The reaction produces a targeted lipid nanoparticle that can be subject to additional steps, e.g., filtration (e.g., tangential flow filtration and/or bulk filtration).

FIG. 4 is a chart outlining various physical parameters, including size, zeta potential (mV), and encapsulation efficiency (%) of targeted lipid nanoparticles produced by a process of the disclosure. The described particles were produced by insertion of a fragment antigen-binding (Fab) targeting moiety into lipid nanoparticle(s) (LNP) encapsulating reporter nucleic acid cargos (GFP-mRNAs or luciferase-mRNAs) as described herein.

FIG. 5 is a chart outlining mean FITC-A expression in Bend3 murine cell line as a function of mRNA dose and Fab to mRNA ratio. The in-vitro results were obtained with lipid nanoparticle (LNP) encapsulating reporter nucleic acid cargos (GFP-mRNAs).

FIG. 6 is a chart outlining mean FITC-A expression in hCMEC cell lines as a function of mRNA dose and Fab to mRNA ratio. The in-vitro results were obtained with lipid nanoparticle (LNP) encapsulating reporter nucleic acid cargos (GFP-mRNAs).

FIGS. 7A-7B are charts outlining mean FITC-A expression in hCMEC cell lines as a function of mRNA dose. FIGS. 7A-7B provide an expanded grafting density impact assessment.

FIG. 8 is a chart outlining the average diameter (nm) of targeted lipid nanoparticles encapsulating a nucleic acid encoding a cargo in various buffers. The Y-axis depicts the impact in the size of the targeted lipid nanoparticle (nm) as a function of repeated freeze thaw (F/T) cycles.

FIG. 9 is a chart outlining the percent (%) encapsulation efficiency of targeted lipid nanoparticles encapsulating a nucleic acid encoding a cargo in various buffers. The Y-axis depicts the impact in the size of the targeted lipid nanoparticle (nm) as a function of repeated freeze thaw (F/T) cycles.

FIG. 10 is a schematic of the insertion of a targeting moiety into a lipid nanoparticle via DSPE-PEG-Maleimide linker lipid micelles and steps of the process development.

FIGS. 11A-11B are charts evaluating the impact of various buffers on the efficacy of the targeted lipid nanoparticle encapsulating a nucleic acid encoding a cargo on human T-cells itself as measured by the mean fluorescence intensity of the cargo protein encoded by the nucleic acid cargo and expressed by the cells.

FIGS. 12A-C are charts outlining various biophysical properties of lipid nanoparticles manufactured in distinct buffers. FIG. 12A is a chart outlining the percent (%) distribution of various lipids in a targeted lipid nanoparticle. FIGS. 12A-B are charts outlining the integrity of the mRNA cargos after insertion of a targeting moiety when heating is used FIG. 12B and when heating is not FIG. 12C used in the reaction.

FIGS. 13A-13B are charts outlining various biophysical properties of lipid nanoparticles manufactured as described herein. FIG. 13A is a chart outlining the thermostability of the targeted lipid nanoparticle encapsulating a nucleic acid encoding a cargo in various buffers. As shown in FIG. 13A T0=time zero, T5D/2-8° C. (thermostability for 5 days between 2-8° C.), and T5D/25 (thermostability for 5 days at 25° C.). FIG. 13B is a chart outlining the relationship between the thermostability of the lipid nanoparticles and the percent encapsulation efficiency of the targeted lipid nanoparticle encapsulating a nucleic acid encoding a cargo in various buffers.

FIGS. 14A, 14B and 14C are charts evaluating the efficacy of lipid nanoparticles manufactured as described herein. FIG. 14A is a chart depicting the impact of various freeze thaw (F/T) cycles. FIG. 14B depicts the thermostability of the nanoparticle at T5D/25 (thermostability for 5 days at 25° C.). FIG. 14C depicts the thermostability of the nanoparticle for 5 days between 2-8° C.).

FIGS. 15A-15B are charts evaluating the impact of temperature, incubation time, and mixing in processes for insertion of targeting moieties into lipid nanoparticles.

FIGS. 16A, 16B, and 16C are charts evaluating the impact of pre-heating in processes for insertion of targeting moieties into lipid nanoparticles.

FIGS. 17A, 17B, and 17C are charts evaluating the impact of targeting moiety incubation time and temperature in processes for insertion of targeting moieties into lipid nanoparticles.

FIGS. 18A, 18B, 18C, and 18D are charts evaluating processes for insertion of targeting moieties into lipid nanoparticles with different lipid types.

FIG. 19 is a chart evaluating the effects of antigen binding protein to LNP ratio.

FIG. 20A is a chart showing the average particle diameter (nanometers) measured before and after Fab-PEG micelle insertion. Particle size remains essentially unchanged across all ethanol concentrations, indicating structural robustness both pre- and post-insertion.

FIG. 20B is a chart showing the polydispersity index (PDI) before and after Fab insertion. Low PDI values at every ethanol concentration confirm a narrow size distribution and minimal aggregation throughout the insertion process.

It should be understood that the drawings and pictures are not necessarily to scale.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Terminology

As used herein, the term “about” and the term “approximately,” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself. Thus, by way of example, “about 10” should be understood to mean “10” and a range no larger than “9-11.” For clarity, as used herein, designation of a range of values includes all integers within or defining the range, and all subranges defined by integers within the range.

As used herein, the term “and/or” refers to and encompasses any and all possible combinations of one or more of the associated listed items, as well as the lack of combinations when interpreted in the alternative (“or”).

As used herein, the term “or” refers to any one member of a particular list and also includes any combination of members of that list. That is, the term “or” shall be interpreted to carry both conjunctive and disjunctive meanings, unless any such meaning is impossible. For instance, “A, B, or C” shall be interpreted to mean A alone, B alone, C alone, A and B but not C, A and C but not B, B and C but not A, and all of A, B, and C, unless any such meaning is impossible.

The singular forms of the articles “a,” “an,” and “the” include plural references unless the context clearly dictates otherwise. For example, the term “a pest” or “at least one pest” can include a plurality of proteins, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the phrase “lipid particle” refers to a lipid composition that can be used to encapsulate a therapeutic nucleic acid (e.g., mRNA).

As used herein, the phrase “targeted lipid nanoparticle” refers to a lipid composition that comprises a functional targeting moiety(ies) for preferentially targeting the targeted lipid nanoparticle and its contents-often a therapeutic nucleic acid (e.g., mRNA)—to a target site of interest (e.g., cell, tissue, organ, and the like).

As used herein, the phrase “Lipid 1” refers to the following lipid:

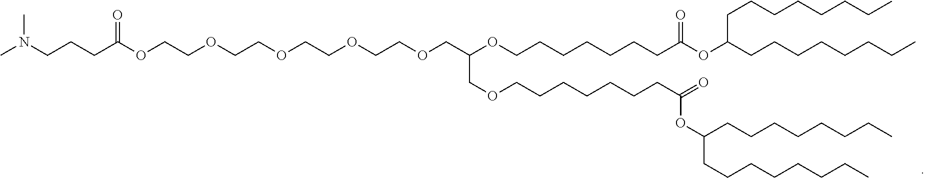 [SMILES: CCCCCCCCC(CCCCCCCC)OC(=O)CCCCCCCOCC(COCCOCCOCCOCCOC(=O)CCCN(C)C)OCCCCCCCC(=O)OC(CCCCCCCC)CCCCCCCC]

As used herein, the phrase “Lipid 2” refers to the following lipid:

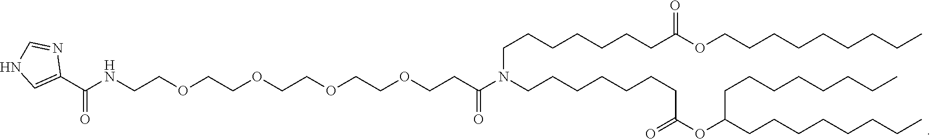 [SMILES: CCCCCCCCCOC(=O)CCCCCCCN(CCCCCCCC(=O)OC(CCCCCCCC)CCCCCCCC)C(=O)CCOCCOCCOCCOCCNC(=O)c1c[nH]cn1]

As used herein, the phrase “Lipid 3” refers to the following lipid:

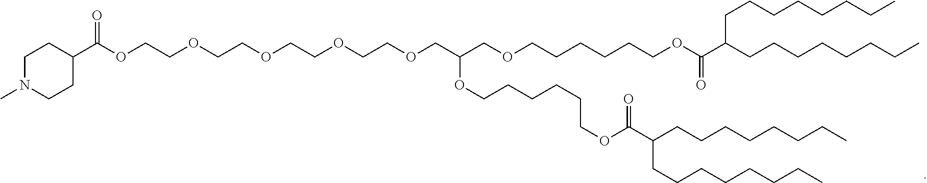 [SMILES: CCCCCCCCC(CCCCCCCC)C(=O)OCCCCCCOCC(COCCOCCOCCOCCOC(=O)C1CCN(C)CC1)OCCCCCCOC(=O)C(CCCCCCCC)CCCCCCCC]

As used herein, the phrase “Lipid 4” refers to the following lipid:

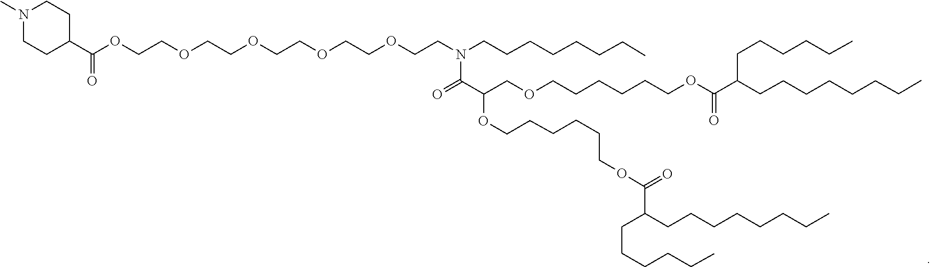 [SMILES: CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCOC(=O)C1CCN(C)CC1]

As used herein, the phrase “Lipid 5” refers to the following lipid:

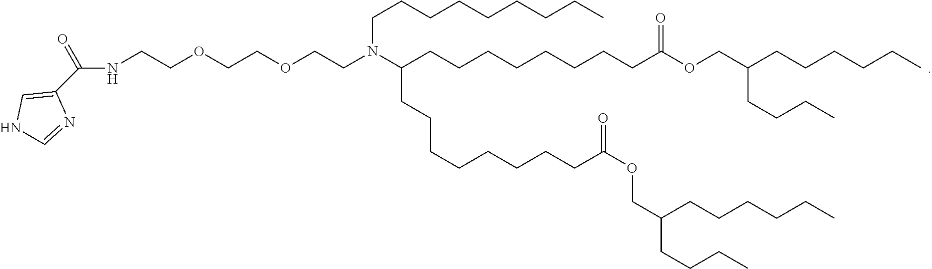 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC]

As used herein, the phrase “Lipid 6” refers to the following lipid:

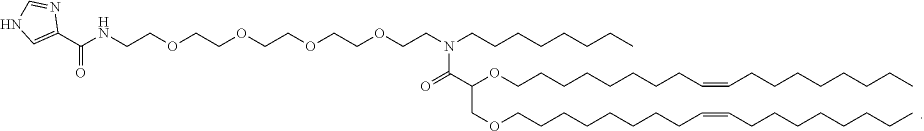 [SMILES: CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1]

As used herein, the phrase “Lipid 7” refers to the following lipid:

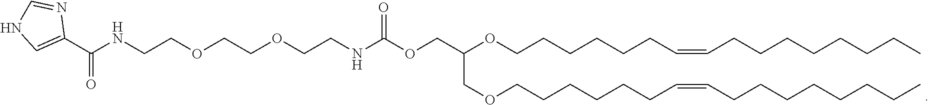 [SMILES: CCCCCCCC/C=C\CCCCCCOCC(COC(=O)NCCOCCOCCNC(=O)c1c[nH]cn1)OCCCCCC/C=C\CCCCCCCC]

As used herein, the phrase “Lipid 8” refers to the following lipid:

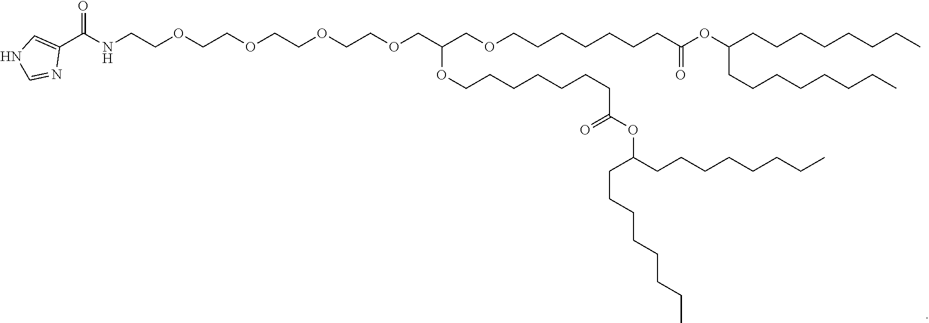 [SMILES: CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

As used herein, the phrase “Lipid 9” refers to the following lipid:

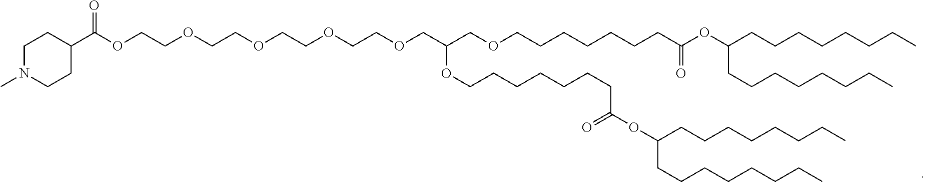 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC]

As used herein, the phrase “Lipid 10” refers to the following lipid:

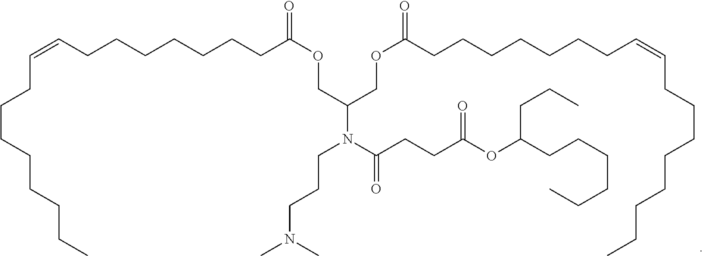 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

As used herein, the phrase “targeting moiety(ies),” includes proteins (such as antibodies and their fragments), peptides, nucleic acids (e.g., aptamers, mRNA, gene editing systems comprising a CRISPR-Cas complex), and/or small molecules, with an affinity for one or more specific target molecules. As used here, a targeting moiety can be inserted into a lipid nanoparticle, thereby producing a targeted lipid nanoparticle.

As used herein, the phrase “specifically binds”, “specific binding”, “specifically binding” or “binds” refers to an affinity of a targeted lipid nanoparticle to a target, e.g., measured either with in-vitro assays or within the antigen with greater affinity than for other antigens. Typically, the targeted lipid nanoparticle binds to its target with an equilibrium dissociation constant (KD) of about 1×10⁷ M or less, for example about 5×10⁸ M or less, about 1×10⁸ M or less, about 1×10⁹ M or less, about 1×10¹⁰ M or less, about 1×10¹¹ M or less, or about 1×10¹² M or less, typically with the KD that is at least one hundred fold less than its KD for binding to a non-specific antigen (e.g., KD of a lipid nanoparticle compared to the KD of a targeted lipid nanoparticle).

As used herein, the phrase “nucleic acid cargo,” as used herein, is intended to include DNA molecules and RNA molecules. A nucleic acid molecule may be single-stranded or double-stranded, and may be modified mRNA (i.e., mRNA comprising non-canonical nucleic acids).

As used herein, the phrase “CAR-T” or “Chimeric Antigen Receptor T” molecule refers to generically engineered proteins that target specific antigens, such as those found in cancer cells.

As used herein, the phrase “antigen-binding protein” includes a protein having at least one CDR and that is capable of selectively recognizing an antigen, i.e., is capable of binding an antigen with a KD that is at least in the micromolar range. Therapeutic antigen-binding proteins (e.g., therapeutic antibodies) frequently require a KD that is in the nanomolar or the picomolar range. “Antigen-binding protein” also includes a protein comprising a first and a second CH3 domain as described herein and a first protein or ligand recognition domain and a second protein or ligand recognition domain, wherein the first protein or ligand recognition domain and the second protein or ligand recognition domain each independently recognize the same protein or ligand, or together recognize the same protein or ligand, or each independently recognize a different protein or ligand. One example of such protein is an immunoadhesin, comprising a fusion protein (hetero- or homo-) dimer wherein the polypeptides of the dimer are fusion polypeptides that comprise a receptor component or a ligand component, wherein the ligand component comprises an amino acid sequence that binds a receptor.

As used herein, the phrase “immunoglobulin single variable domain (ISVD),” refers to a binding protein that has a VHH sequence of amino acids or a fragment or variant thereof, from a heavy chain only antibody. For instance, an ISVD of the type obtainable from Camelids may be referred to herein in the alternative, as a “VHH domain” or fragment thereof or as a “Nanobody”. An ISVD can be derived from certain species of shark (for example, “IgNAR domains”). It must be noted that NANOBODY® and NANOBODIES® are registered trademarks of Ablynx N. V. An ISVD can comprise the following structure: FR1-CDR-FR2-CDR-FR3-CDR-FR4, wherein CDR 1-3 refer to complementarity determining regions 1-3 and FR1-4 refer to framework regions 1 to 4.

As used herein, the phrase “fragment antigen-binding” or “Fab” is a particular antigen-binding protein composed of one constant and one variable domain of each of the heavy and the light chain. The variable domain contains the paratope (the antigen-binding site), comprising a set of complementarity-determining regions, at the amino terminal end of the monomer. Each arm of the Y thus binds an epitope on the antigen.

As used herein, the phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

As used herein, the phrase “Fc-containing protein” includes antibodies, bispecific antibodies, immunoadhesins, and other binding proteins that comprise at least a functional portion of an immunoglobulin CH2 and CH3 region and can serve as targeting moiety(ies) when incorporated into the lipid nanoparticle. A “functional portion” refers to a CH2 and CH3 region that can bind a target, e.g., a Fc receptor, and/or that can participate in the activation of complement. If the CH2 and CH3 region contains deletions, substitutions, and/or insertions or other modifications that render it unable to activate complement, the CH2 and CH3 region is not functional, but in some instances, a non-functional Fc-containing protein may suffice for targeting purposes.

As used herein, the term “antibody”, as used herein, includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10-9 M, at least 10-10 M; at least 10-11 M; or at least 10-12 M, as measured by surface plasmon resonance, e.g., BIACORE™ or solution-affinity ELISA.

As used herein, the term “cell” includes any cell that has a binding receptor for a particular targeting ligand. In some embodiments, as used herein, a cell is used as an expression vehicle for the cargo within the targeted lipid nanoparticles and/or the lipid nanoparticles (e.g., a hCMEC cell, Bend3 cells, hematopoietic stem cells, and/or T-cells). In some embodiments, the cell is a human, monkey, ape, hamster, rat, or mouse cell. In some embodiments, the cell is eukaryotic and is selected from the following cells: brain microvascular endothelial cell line (hCMEC) cell, bEnd.3 cells, myeloma cell, tumor cell, and a cell line derived from an aforementioned cell.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation. For example, where the disclosure describes a “lipid nanoparticle,” or a “targeted lipid nanoparticle,” without a “cargo,” this is also intended to provide antecedent basis for a negative limitation.

As used herein, the phrase “sequence identity” or “identity” in the context of two polynucleotide sequences makes reference to the residues in the two sequences that are the same when aligned for maximum correspondence over a specified comparison window.

As used herein, the phrase “percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, the term “homology” pertains to comparative studies. Homology indicates an ancient common origin and temporal evolution and refers to structural characteristics. In comparative anatomy, it is used to compare structures in different animal species. In comparative protein biochemistry, “homology” retains the original meaning of “having a common evolutionary origin” and is used to evolutionarily define two or more proteins by locating common structural characteristics and common spatial distribution of, for instance, beta strands, helices, and folds. Accordingly, homologous protein structures are defined by spatial analyses. Measuring structural homology involves computing the geometric-topological features of a space. One approach used to generate and analyze three-dimensional (3D) protein structures is homology modeling (also called comparative modeling or knowledge-based modeling). Homology modeling works by finding similar sequences on the basis of the obvious fact that 3D similarity reflects 2D similarity. Nonetheless, it is important to note that homologous structures do not imply sequence similarity as a necessary condition. Sequence identity is the amount of characters which match exactly between two different sequences. Hereby, gaps are not counted and the measurement is relational to the shorter of the two sequences. This has the effect that sequence identity is not transitive, i.e. if sequence A=B and B=C then A is not necessarily equal C (in terms of the identity distance measure): A: AAGGCT, B: AAGGC, C: AAGGCAT/Here identity (A,B)=100% (5 identical nucleotides/min (length (A),length (B))). Identity (B,C)=100%, but identity (A,C)=85% ((6 identical nucleotides/7)). So 100% identity does not mean two sequences are the same. Sequence similarity is first of all a general description of a relationship but nevertheless it's more or less common practice to define similarity as an optimal matching problem (for sequence alignments or unless defined otherwise). Hereby, the optimal matching algorithm finds the minimal number of edit operations (inserts, deletes, and substitutions) in order to transform the one sequence into an exact copy of the other sequence being aligned (edit distance). Using this, the percentage sequence similarity of the examples above are sim (A,B)=60%, sim (B,C)=60%, sim (A,C)=86% (semi-global, sim=1−(edit distance/unaligned length of the shorter sequence)). But there are other ways to define similarity between two objects (e.g. using tertiary structure of proteins).

As used herein, “statistically significant” indicates a statistical correlation having a p-value ≤0.05.

### DETAILED DESCRIPTION

### I. Overview

All of the functionalities described in connection with one embodiment of the methods, compositions, or formulations described herein are intended to be applicable to the additional embodiments of the methods, compositions, or formulations described herein except where expressly stated or where the feature or function is incompatible with the additional embodiments. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

Encapsulation of nucleic acid cargos into therapeutic nanoparticles is critical to prevent their degradation by nucleases upon administration to a subject. Many lipid nanoparticles (LNP) currently used in commercial settings are composed of cholesterol, a helper lipid, a PEGylated lipid and an ionizable amine-containing lipid. Within these nanoparticles, cholesterol and helper lipids typically support the maintenance of structural integrity, while PEGylated lipids ensure colloidal stability and reduce accumulation in the reticuloendothelial system (RES). The ionizable amine-containing lipid is believed to form a complex with a nucleic acid cargo. The ionizable nature of this lipid is relevant not only for the encapsulation of the nucleic acids cargo within the complex, but also during delivery of the cargo to a subject. This is because the ionizable lipid generally only becomes charged at non-physiological pH (pKa 6-7), remaining uncharged while in circulation within the subject. Upon cell uptake and lysosomal localization, the ionizable lipid becomes charged at the low lysosomal pH. This, combined with the unique features of other lipids, facilitates lysosomal escape and enables mRNA expression or siRNA gene silencing, or another form of gene expression from the nucleic acid cargo. However, an understanding of how even widely used formulations distribute within a subject's body upon administration remains limited. Existing passive targeting approaches suffer from several limitations.

An effective approach to overcoming these limitations involves attaching targeting moieties to the surfaces of nanoparticles, a process that produces “targeted lipid nanoparticles”. These targeted lipid nanoparticles can then bind to, e.g., specific target cells through ligand-receptor interactions, triggering receptor-mediated endocytosis and subsequent drug release within the cell. For higher efficiency of targeting, including higher binding and internalization of the nucleic acid cargo, the targeting moieties incorporated into the lipid nanoparticles can be, e.g., receptors expressed on the surface of target cancer cells. Such targeted delivery strategy may achieve high specificity and efficiency while minimizing nonspecific binding and the multidrug resistance efflux mechanism. It is therefore important to understand the process by which targeting moiety can be incorporated into lipid nanoparticles, thereby providing targeted lipid nanoparticles.

Provided herein are methods for inserting a targeting moiety (such as a fragment antigen-binding moiety, or a chimeric antigen receptor moiety, or the like) into a lipid nanoparticle and compositions resulting from such methods. A nucleic acid cargo can be pre-encapsulated into the lipid nanoparticle prior to the insertion. The insertion of the targeting moiety into the lipid nanoparticle itself can be performed under conditions that do not fragment the nucleic acid cargo. The methods described herein generally combine in a temperature-controlled reaction suitable amounts of a targeting moiety with suitable amounts of a lipid nanoparticle under controlled temperature conditions, for time periods no longer than 4 hours, optionally without any shaking. A quenching operation is then provided by rapidly cooling the reaction to about 20° C. or lower. The process yields greater than 80% of targeted lipid nanoparticles having substantially unfragmented nucleic acid cargos therein.

Subsequent to cooling, the lipid nanoparticles can be subject to filtration and packaging. The process described herein produces lipid nanoparticle with

Additional details are provided in the following sections.

### II. Components of the Targeted Lipid Nanoparticle

In many instances, the disclosure provides a targeted lipid nanoparticle produced by a process comprising the steps of combining in a, e.g., soluble reaction from 0.15 μg of the targeting moiety per gram of the total lipid nanoparticle to 30 g of the targeting moiety per gram of the total lipid nanoparticle for a time-period of at least one hour at in a lipid nanoparticle having a nucleic acid cargo encapsulated therein, whereby the soluble reaction is conducted for a time-period of at least one hour at a temperature no higher than 42° C., thereby producing an amount of targeted lipid nanoparticles whereby the nucleic acid cargo within the targeted lipid nanoparticles is less than 20% degraded compared to a nucleic acid cargo in a particle where the targeting moiety is not inserted by the process. In many instances, the lipid nanoparticle comprises an amount of an ionizable lipid, an amount of cholesterol, an amount of neutral lipid, and an amount of a PEG-lipid.

### Ionizable Cationic Lipids

Lipid nanoparticles and targeted lipid nanoparticles of the disclosure generally comprise an amount of an ionizable lipid, an amount of cholesterol, an amount of neutral lipid, and an amount of a PEG-lipid. In most instances, such particles encapsulate a pre-packaged cargo (e.g., a nucleic acid cargo). Lipid nanoparticles and targeted lipid nanoparticles of the disclosure generally comprise ionizable cationic lipids with pKa values typically ranging from 5 to 8. Depending on the number of amino heads, ionizable cationic lipids can be classified as either monoamino or polyamino lipids. Non-limiting examples of ionizable cationic lipids contemplated in particles of the disclosure include a Lipid 1, Lipid 2, Lipid 3, Lipid 4, Lipid 5, Lipid 6, Lipid 7, Lipid 8, Lipid 9, Lipid 10, KC2, DLin-MC3-DMA (MC3), SM-102, and ALC-0315. Non-limiting examples of monoamino ionizable cationic lipids contemplated in particles of the disclosure include 306O i10, C12-200, 5A2-SC8, TT3, FTT5. Non-limiting examples of polyamino ionizable cationic lipids contemplated in particles of the disclosure include cKK-E12 and OF-02. In many instances, the ionizable lipid is selected from the group consisting of lipid 2, lipid 3, lipid 4, lipid 5, KC2, DLin-MC2-DMA, DLin-MC3-DMA, DSDMA, DODMA, DLinDMA, DLenDMA, γ-DLenDMA, DLin-K-DMA, DLin-C2K-DMA, DLin-K-C3-DM A, DLin-K-CA-DMA, DLen-C2K-DMA, γ-DLen-C2K-DMA, or DLin-MP-DMA. In some embodiments, the ionizable lipid is a lipid disclosed in WO2022013443 or WO2023135305.

In some embodiments that can be combined with any of the above embodiments, the ionizable lipid is one or more lipids selected from the group consisting of the following:

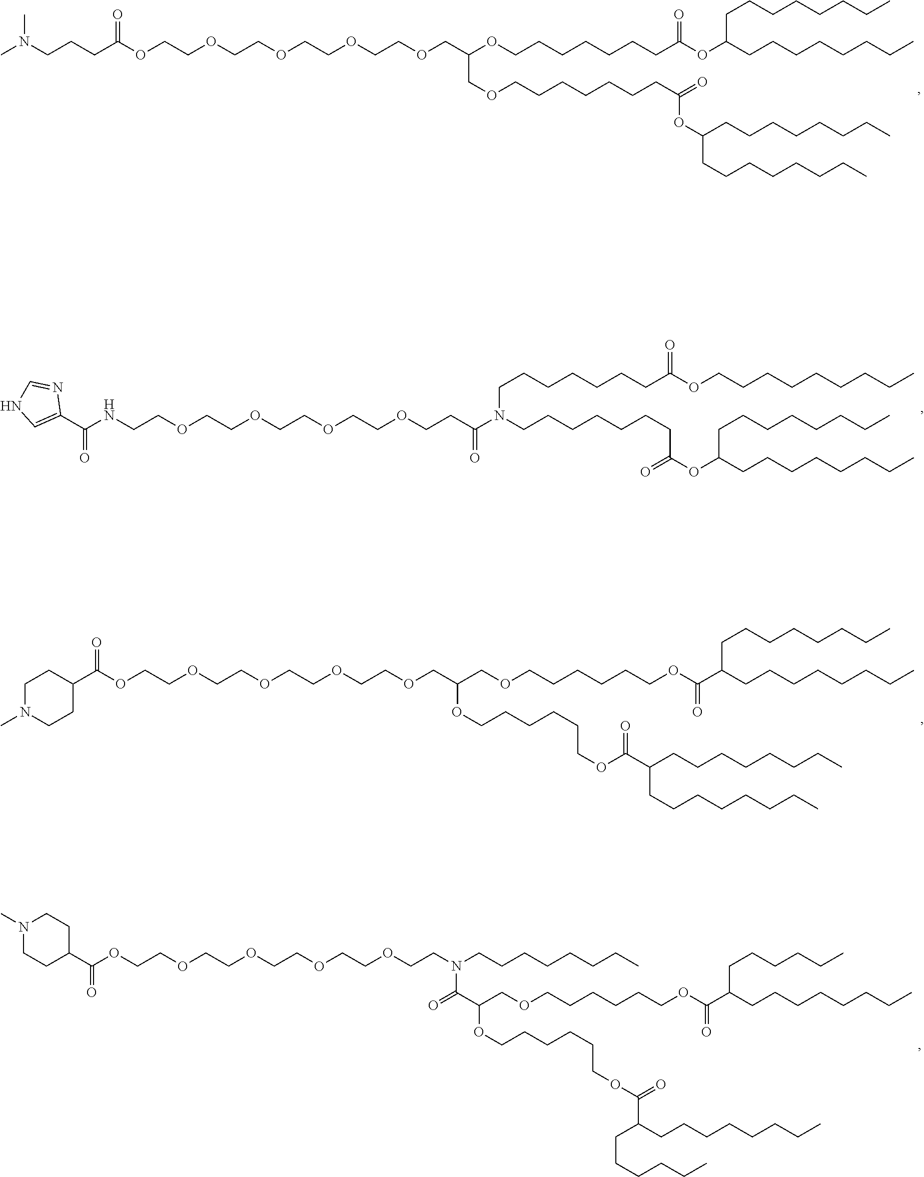

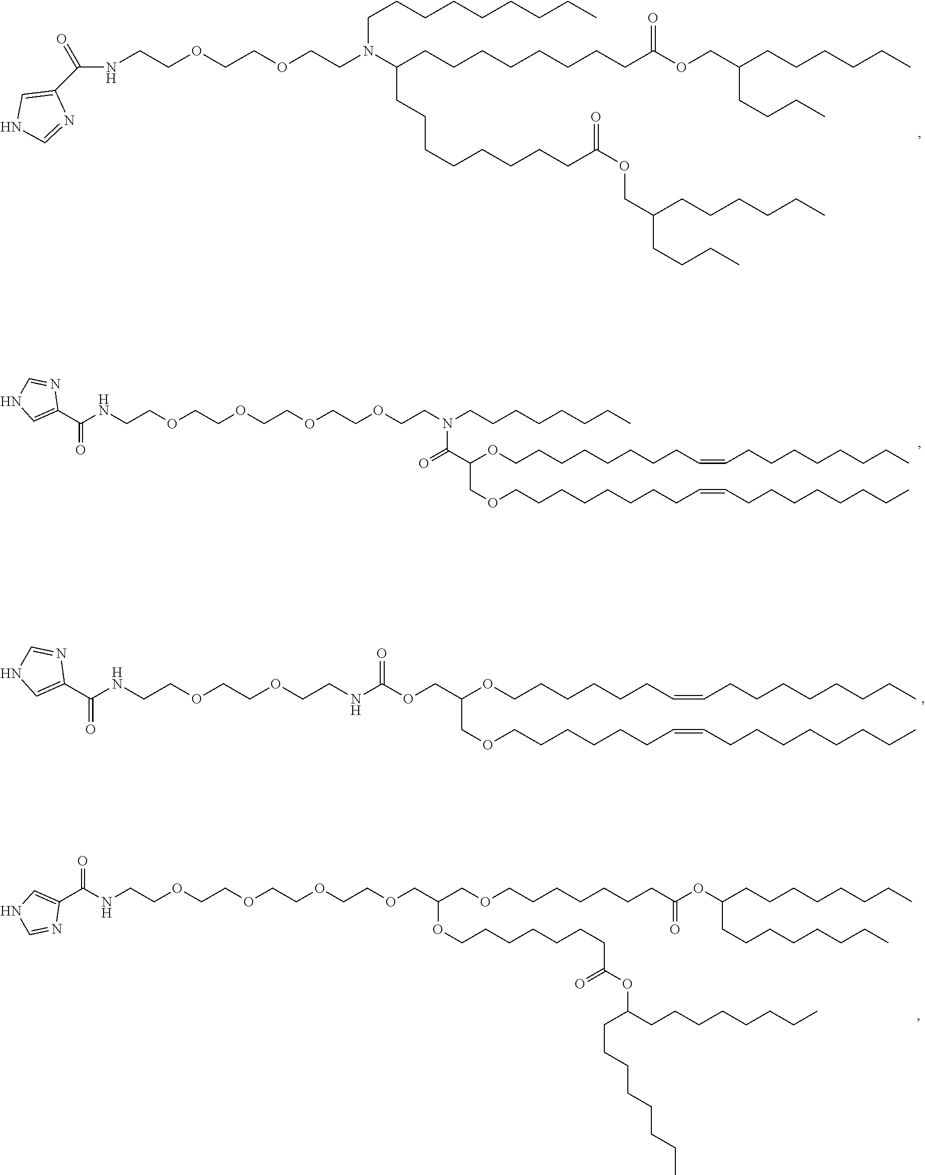 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC ; CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1 ; CCCCCCCC/C=C\CCCCCCOCC(COC(=O)NCCOCCOCCNC(=O)c1c[nH]cn1)OCCCCCC/C=C\CCCCCCCC ; CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

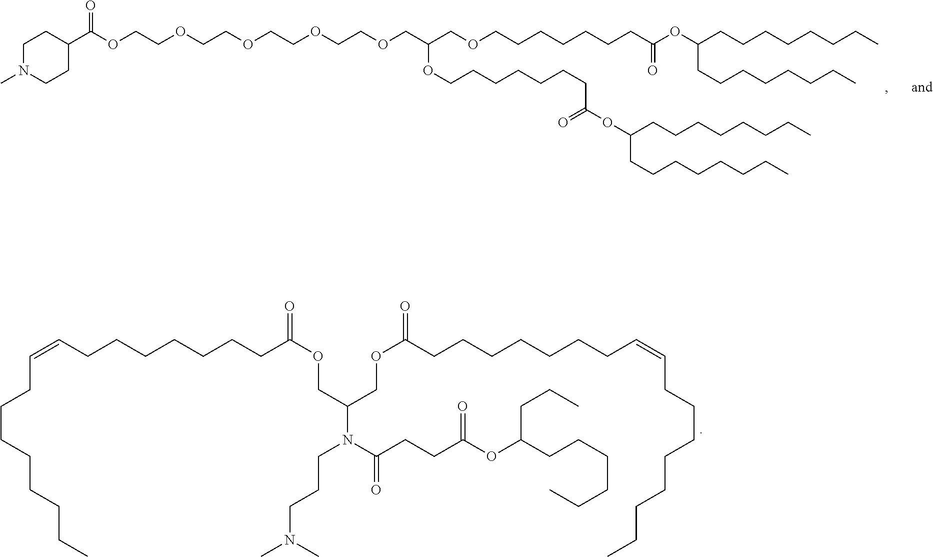 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC ; CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

Ionizable lipids for use with the instant LNPs can have the general structure—a polar head group, a hydrophobic tail region (either branched or unbranched), and a linker between the two domains, such as the lipids illustrated herein.

### PEG-Lipids

A variety of PEG lipids are contemplated for use with the LNPs of the disclosure, including terminally modified PEG lipids. PEG lipids for use in the present invention can be, for example, maleimide terminally modified PEG lipids that can be conjugated with cell targeting peptides. PEG lipids for use with the instant LNPs can have the general structure —(CH₂CH₂O)ₙ— or —(CH₂CH₂O)ₙCH₂CH₂. This general structure can further be modified with heterobifunctional maleimide linker. The disclosure contemplates that a variety of PEG molecules can be incorporated into its LNPs, including polyalkylene glycols, polypropylene or polybutylene glycols, methoxy poly(ethylene glycol), or methoxy poly(ethylene glycol) propionic acid (mPEG-acid) where n can be from about 1 to about 400. An LNP comprising a thiol reactive motive conjugated to a PEG molecule (e.g., heterobifunctional maleimide PEG) can be decorated with various types of peptides that are displayed on the surface of the LNP molecule.

In some instances, a PEG molecule is linked to a thiol reactive group for further conjugation to a peptide. In some instances the PEG molecule is a maleimide conjugated PEG molecule. Reactive PEGs can be used for amine pegylation, thiol pegylation, or N-terminal pegylation. The amine in the N-terminus and/or the carboxyl group in the C-terminus can react with a targeting peptide. In some instances the PEG molecule is methoxy poly(ethylene glycol) succinimidyl proprionate (mPEG-SPA). In some instances, a PEG molecule is a methoxy poly (ethylene glycol) propionic acid (mPEG-acid). In some cases, the polyethylene glycol molecule weighs from about 500 kilodaltons to about 5000 kilodaltons. The covalent attachment of a targeting peptide to an LNP via a thiol reactive linkage can change the physicochemical characteristics of the LNP. Examples of physicochemical characteristics that can be altered by binding to a PEG include its zeta potential, its polydispersity index (PDI), and the overall hydrodynamic size of the LNP.

Non-limiting examples of commercially available PEGs suitable for use in the invention include, but are not limited to those available from Nektar Therapeutics, San Carlos, CA, such as mPEG-NH₂ (Mw about 10 kDa, about 20 kDa), methoxy PEG Succinimidyl α-Methylbutanoate (SMB), SMB-PEG-SMB, methoxy PEG Succinimidyl Propionate (mPEG-SPA), Branched PEG N-Hydroxysuccinimide (mPEG2-NHS), mPEG-CM-HBA-NHS, NHS-HBA-CM-PEG-CM-HBA-NHS, mPEG-ButyrALD, ButyrALD-PEG-ButyrALD, Branched PEG ButyrALD (mPEG2-ButyrALD), Ortho-pyridylthioester (mPEG-OPTE), mPEG Maleimide (MAL), MAL-PEG-MAL, Branched PEG Maleimide (mPEG2-MAL), Forked Maleimide (mPEG-MAL2 and mPEG2-MAL2), mPEG-Ortho-pyridyldisulfide (mPEG-OPSS), OPSS-PEG-OPSS, mPEG-SH, SH-PEG-SH, Amine-PEG-Acid, Boc-PEG-NHS, Fmoc-PEG-NHS, MAL-PEG-NHS, Vinylsulfone-PEG-NHS, Acrylate-PEG-NHS Ester.

Non-limiting examples of PEGs that can be used in amine pegylation include, for example, PEGs manufactured by Jenken Technology USA such as: Y-shape PEG NHS Esters, Y-shape PEG Carboxyl, Glucose PEG NHS Ester, Galactose PEG NHS Ester, Methoxy PEG Succinimidyl Carboxymethyl Ester, Methoxy PEG Carboxyl, Methoxy PEG Succinimidyl Butanoate, Methoxy PEG Succinimidyl Hexanoate, Methoxy PEG Hexanoic Acid, Methoxy PEG Succinimidyl Succinamide, Methoxy PEG Succinimidyl Glutaramide, Methoxy PEG Succinimidyl Carbonate, Methoxy PEG Nitrophenyl Carbonate, Methoxy PEG Succinimidyl Succinate, Methoxy PEG Succinimidyl Glutarate. Non-limiting examples of PEGs that can be used in thiol pegylation include Y-shape PEG Maleimide, Methoxy PEG Maleimide, Methoxy PEG Vinylsulfone, Methoxy PEG Thiol. Non-limiting examples of PEGs that can be used in N-terminal pegylation include, for example, PEGs manufactured by Jenken Technology USA such as: Y-shape PEG Aldehyde, Y-shape PEG Acetaldehyde, Y-shape PEG Propionaldehyde, Methoxy PEG Propionaldehyde.

In some cases, the molecular weight of a PEG molecule can be greater than 500 daltons (Da), greater than 1 kilodalton (kDa), greater than 5 kilodaltons (kDa), greater than 10 kilodaltons (kDa), greater than 15 kilodaltons (kDa), greater than 20 kilodaltons (kDa), greater than 25 kilodaltons (kDa), greater than 30 kilodaltons (kDa), greater than 35 kilodaltons (kDa), greater than 40 kilodaltons (kDa), greater than 45 kilodaltons (kDa), greater than 50 kilodaltons (kDa), greater than 55 kilodaltons (kDa), greater than 60 kilodaltons (kDa), greater than 65 kilodaltons (kDa), greater than 70 kilodaltons (kDa), greater than 75 kilodaltons (kDa), greater than 80 kilodaltons (kDa), greater than 85 kilodaltons (kDa), greater than 90 kilodaltons (kDa), greater than 95 kilodaltons (kDa), greater than 100 kilodaltons (kDa).

In some cases the molecular weight of a PEG oligomer can be from about 1 kilodalton (kDa) to about 5 kilodaltons (kDa), from about 1 kilodalton (kDa) to about 10 kilodaltons (kDa), from about 10 kilodaltons (kDa) to about 20 kilodaltons (kDa), from about 10 kilodaltons (kDa) to about 30 kilodaltons (kDa), from about 10 kilodaltons (kDa) to about 40 kilodaltons (kDa), from about 10 kilodaltons (kDa) to about 50 kilodaltons (kDa), from about 20 kilodaltons (kDa) to about 30 kilodaltons (kDa), from about 20 kilodaltons (kDa) to about 40 kilodaltons (kDa), from about 20 kilodaltons (kDa) to about 50 kilodaltons (kDa), from about 30 kilodaltons (kDa) to about 40 kilodaltons (kDa), from about 30 kilodaltons (kDa) to about 50 kilodaltons (kDa).

### Neutral-Lipids—Helper Lipids—Phospholipids

Phospholipids are neutral “helper” lipids that contribute to the formation of lipid nanoparticles and the escape of endosomes. In many instances, a particle of the disclosure comprises a neutral lipid that is a phosphatidylcholine lipid or a phosphatidylethanolamine lipid. The phosphatidylcholine lipid or the phosphatidylethanolamine lipid can be selected from the group consisting of DSPC, DPPC, and POPC.

### Cholesterol

The inclusion of cholesterol in nucleic acid-containing LNP formulations is based primarily on two major findings obtained with liposomal formulations of small molecule therapeutics: 1) cholesterol is an exchangeable molecule that can accumulate within liposomes during circulation, 2) cholesterol dramatically reduces the amount of surface-bound proteins and improves the circulating half-life.

### Targeting Moieties

As described herein, targeting moieties are proteins (mainly antibodies and their fragments), peptides, nucleic acids (aptamers), small molecules, or others (vitamins or carbohydrates) that are either attached to a surface of a lipid nanoparticle or otherwise incorporated into the structure of a lipid nanoparticle, thus providing a targeted lipid nanoparticle. The affinity of a targeting moiety present in a targeting lipid nanoparticle of the disclosure and, e.g., a receptor on the surface of a cell and/or tissue can be an affinity of at least 10¹, 10², 10³, 10⁴, 10⁵, 10⁶, 10⁷, 10⁸, 10⁹, or 10¹⁰ M⁻¹. In some instances, the targeting moiety is antigen binding protein, an antibody fragment, a VHH domain, or a fragment thereof that is sufficient for binding to a cell surface receptor in vivo.

In many instances, the lipid nanoparticle comprises amounts of the ionizable lipid, the cholesterol, the neutral lipid, and the PEG-lipid at molar ratio ranges respectively of 40%-60% ionizable lipid, 20%-35% cholesterol, 10%-30% of neutral lipid, 0.5%-5% of PEG-lipid. In certain embodiments, the ionizable, cationic lipid described herein may be present in the total lipid amount in a range of 30-70 mole percent, 30-60 mole percent 30-50 mole percent 40-70 mole percent, 40-60 mole percent, 40-50 mole percent 50-70 mole percent, 50-60 mole percent, or of about 30 mole percent, about 35 mole percent, about 40 mole percent, about 45 mole percent, about 50 mole percent, about 55 mole percent, about 60 mole percent, about 65 mole percent, or about 70 mole percent. The cholesterol may be present in the total lipid amount in a range of 20-70 mole percent, 20-60 mole percent, 20-50 mole percent, 30-70 mole percent, 30-60 mole percent, 30-50 mole percent, 40-70 mole percent, 40-60 mole percent, 40-50 mole percent, 50-70 mole percent, 50-60 mole percent, or about 20 mole percent, about 25 mole percent, about 30 mole percent, about 35 mole percent, about 40 mole percent, about 45 mole percent, The neutral lipid may be present in the total lipid in a range of 1-10 mole percent, 1-15 mole percent, 1-12 mole percent, 1-10 mole percent, 3-15 mole percent, 3-12 mole percent, 3-10 mole percent, 4-15 mole percent, 4-12 mole percent, 4-10 mole percent, 4-8 mole percent, 5-15 mole percent, 5-12 mole percent, 5-10 mole percent, 6-15 mole percent, 6-12 mole percent, 6-10 more percent, or about 1 mole percent, about 2 mole percent, about 3 mole percent, about 4 mole percent, about 5 mole percent, about 6 mole percent, about 7 mole percent, about 8 mole percent, about 9 mole percent, about 10 mole percent, about 11 mole percent, about 12 mole percent, about 13 mole percent, about 14 mole percent, or about 15 mole percent. The PEG-lipid may be present in the total lipid amount in a range of 0.01-10 mole percent, 0.01-5 mole percent, 0.01-4 mole percent, 0.01-3 mole percent, 0.01-2 mole percent, 0.01-1 mole percent, 0.1-10 mole percent, 0.1-5 mole percent, 0.1-4 mole percent, 0.1-3 mole percent, 0.1-2 mole percent, 0.1-1 mole percent, 0.5-10 mole percent, 0.5-5 mole percent, 0.5-4 mole percent, 0.5-3 mole percent, 0.5-2 mole percent, 0.5-1 mole percent 1-2 mole percent, 3-4 mole percent, 4-5 mole percent, 5-6 mole percent, or 1.25-1.75 mole percent. In some embodiments, the PEG-lipid may be about 0.5 mole percent, about 1 mole percent, about 1.5 mole percent, about 2 mole percent, about 2.5 mole percent, about 3 mole percent, about 3.5 mole percent about 4 mole percent, about 4.5 mole percent, about 5 mole percent, or about 5.5 mole percent of the total lipid composition.

In some embodiments, an amount of targeting moiety is added to the reaction mixture and the ranges can be adjusted depending on the targeting moiety. For instances, in some cases from about 0.00015 mg (0.15 μg) per mol to about 30 g per mol of a VHH targeting moiety is added per mol of the total lipids, or from about 0.00015 mg per mol to about 15 g per mol, or from about 0.00015 mg per mol to about 5 g per mol. In some instances, from about 10 mg per mol to about 100 mg per mol, from about 50 mg per mol to about 500 mg per mol, from about 100 mg per mol to about 1 g per mol, from about 200 mg per mol to about 2 g per mol, from about 500 mg per mol to about 5 g per mol, from about 1 g per mol to about 5 g per mol of a targeting moiety (e.g. VHH) is added per mol of total lipids. In some instances, the amount of the VHH targeting moiety in the reaction whereby the ranges from 2.0 g-3.5 g of the VHH targeting moiety per 1 mol of the total lipids in the lipid nanoparticle, for a lipid nanoparticle with 50:38.5:10:1.5 composition (ionizable:cholesterol:helper:PEG lipid). In some instances, from about 0.00015 mg (0.15 μg) per mol to about 30 g per mol of a Fab fragment is added per mol of the total lipids. In some instances, from about 0.00015 mg per mol to about 15 g per mol of a Fab fragment is added per mol of the total lipids, or from about 0.00015 mg per mol to about 5 g per mol of a Fab fragment is added per mol of the total lipids. For instances, in some cases from about 10 mg per mol to about 50 mg per mol, from about 10 mg per mol to about 100 mg per mol, from about 10 mg per mol to about 200 mg per mol, from about 10 mg per mol to about 500 mg per mol, from about 10 mg per mol to about 1 g per mol, from about 20 mg per mol to about 100 mg per mol, from about 50 mg per mol to about 200 mg per mol, from about 50 mg per mol to about 500 mg per mol, from about 100 mg per mol to about 500 mg per mol, from about 100 mg per mol to about 1 g per mol, from about 200 mg per mol to about 1 g per mol, from about 200 mg per mol to about 2 g per mol, from about 500 mg per mol to about 2 g per mol, from about 500 mg per mol to about 5 g per mol, from about 1 g per mol to about 5 g per mol, from about 1 g per mol to about 10 g per mol, or from about 0.00015 mg (0.15 μg) per mol to about 30 g per mol of a Fab targeting moiety is added per mol of the total lipids. In some instances, the amount of the Fab targeting moiety in the reaction whereby the ranges from 1.0 g-10 g, or 1.0 g to 15 g of the Fab targeting moiety per 1 mol of the total lipids in the lipid nanoparticle, for a lipid nanoparticle with a reference 50:38.5:10:1.5 composition (ionizable:cholesterol:helper:PEG lipid).

In some embodiments, an amount of targeting moiety is added in relation to an amount of a nucleic acid cargo that is also added to the reaction mixture. In some embodiments, 1 mg to 400 mg of a targeting moiety is added per gram of a nucleic acid encoding a cargo, 1 mg to 390 mg of a targeting moiety is added per gram of a nucleic acid encoding a cargo, 1 mg to 380 mg of a targeting moiety is added per gram of a nucleic acid encoding a cargo, 1 mg to 370 mg of a targeting moiety is added per gram of a nucleic acid encoding a cargo, 1 mg to 360 mg of a targeting moiety is added per gram of a nucleic acid encoding a cargo, 1 mg to 350 mg of a targeting moiety is added per gram of a nucleic acid encoding a cargo, 1 mg to 340 mg of a targeting moiety is added per gram of a nucleic acid encoding a cargo, 1 mg to 330 mg of a targeting moiety is added per gram of a nucleic acid encoding a cargo, 1 mg to 320 mg of a targeting moiety is added per gram of a nucleic acid encoding a cargo, 1 mg to 310 mg of a targeting moiety is added per gram of a nucleic acid encoding a cargo, 1 mg to 300 mg of a targeting moiety is added per gram of a nucleic acid encoding a cargo, 1 mg to 290 mg of a targeting moiety is added per gram of a nucleic acid encoding a cargo, 1 mg to 280 mg of a targeting moiety is added per gram of a nucleic acid encoding a cargo, 1 mg to 270 mg of a targeting moiety is added per gram of a nucleic acid encoding a cargo, 1 mg to 260 mg of a targeting moiety is added per gram of a nucleic acid encoding a cargo, 1 mg to 250 mg of a targeting moiety is added per gram of a nucleic acid encoding a cargo, 1 mg to 240 mg of a targeting moiety is added per gram of a nucleic acid encoding a cargo, 1 mg to 230 mg of a targeting moiety is added per gram of a nucleic acid encoding a cargo, 1 mg to 220 mg of a targeting moiety is added per gram of a nucleic acid encoding a cargo, 1 mg to 210 mg of a targeting moiety is added per gram of a nucleic acid encoding a cargo, 1 mg to 200 mg of a targeting moiety is added per gram of a nucleic acid encoding a cargo, 1 mg to 190 mg of a targeting moiety is added per gram of a nucleic acid encoding a cargo, 1 mg to 180 mg of a targeting moiety is added per gram of a nucleic acid encoding a cargo, 1 mg to 170 mg of a targeting moiety is added per gram of a nucleic acid encoding a cargo, 1 mg to 160 mg of a targeting moiety is added per gram of a nucleic acid encoding a cargo, 1 mg to 150 mg of a targeting moiety is added per gram of a nucleic acid encoding a cargo, 1 mg to 160 mg of a targeting moiety is added per gram of a nucleic acid encoding a cargo, 1 mg to 150 mg of a targeting moiety is added per gram of a nucleic acid encoding a cargo, 1 mg to 140 mg of a targeting moiety is added per gram of a nucleic acid encoding a cargo, 1 mg to 130 mg of a targeting moiety is added per gram of a nucleic acid encoding a cargo, 1 mg to 120 mg of a targeting moiety is added per gram of a nucleic acid encoding a cargo, 1 mg to 110 mg of a targeting moiety is added per gram of a nucleic acid encoding a cargo, 1 mg to 100 mg of a targeting moiety is added per gram of a nucleic acid encoding a cargo, 1 mg to 90 mg of a targeting moiety is added per gram of a nucleic acid encoding a cargo, 1 mg to 80 mg of a targeting moiety is added per gram of a nucleic acid encoding a cargo, 1 mg to 70 mg of a targeting moiety is added per gram of a nucleic acid encoding a cargo, 1 mg to 60 mg of a targeting moiety is added per gram of a nucleic acid encoding a cargo, 1 mg to 50 mg of a targeting moiety is added per gram of a nucleic acid encoding a cargo, 1 mg to 40 mg of a targeting moiety is added per gram of a nucleic acid encoding a cargo, 1 mg to 30 mg of a targeting moiety is added per gram of a nucleic acid encoding a cargo, 1 mg to 20 mg of a targeting moiety is added per gram of a nucleic acid encoding a cargo, or 1 mg to 10 mg of a targeting moiety is added per gram of a nucleic acid encoding a cargo.

In some embodiments, an amount of targeting moiety is added to the reaction mixture. In some embodiments, an amount of targeting moiety is added per gram of a nucleic acid cargo, such as from 50 mg to 1,400 mg per gram of the nucleic acid cargo. Thus, the ranges include, without limitation, 50 mg to 100 mg, 50 mg to 150 mg, 50 mg to 200 mg, 50 mg to 250 mg, 50 mg to 300 mg, 50 mg to 350 mg, 50 mg to 400 mg, 50 mg to 450 mg, 50 mg to 500 mg, 50 mg to 550 mg, 50 mg to 600 mg, 50 mg to 650 mg, 50 mg to 700 mg, 50 mg to 750 mg, 50 mg to 800 mg, 50 mg to 850 mg, 50 mg to 900 mg, 50 mg to 950 mg, 50 mg to 1,000 mg, 50 mg to 1,050 mg, 50 mg to 1,100 mg, 50 mg to 1,150 mg, 50 mg to 1,200 mg, 50 mg to 1,250 mg, 50 mg to 1,300 mg, 50 mg to 1,350 mg, or 50 mg to 1,400 mg of a targeting moiety per gram of a nucleic acid encoding a cargo; 100 mg to 150 mg, 100 mg to 200 mg, 100 mg to 250 mg, 100 mg to 300 mg, 100 mg to 350 mg, 100 mg to 400 mg, 100 mg to 450 mg, 100 mg to 500 mg, 100 mg to 550 mg, 100 mg to 600 mg, 100 mg to 650 mg, 100 mg to 700 mg, 100 mg to 750 mg, 100 mg to 800 mg, 100 mg to 850 mg, 100 mg to 900 mg, 100 mg to 950 mg, 100 mg to 1,000 mg, 100 mg to 1,050 mg, 100 mg to 1,100 mg, 100 mg to 1,150 mg, 100 mg to 1,200 mg, 100 mg to 1,250 mg, 100 mg to 1,300 mg, or 100 mg to 1,350 mg of a targeting moiety per gram of a nucleic acid encoding a cargo; 150 mg to 200 mg, 150 mg to 250 mg, 150 mg to 300 mg, 150 mg to 350 mg, 150 mg to 400 mg, 150 mg to 450 mg, 150 mg to 500 mg, 150 mg to 550 mg, 150 mg to 600 mg, 150 mg to 650 mg, 150 mg to 700 mg, 150 mg to 750 mg, 150 mg to 800 mg, 150 mg to 850 mg, 150 mg to 900 mg, 150 mg to 950 mg, 150 mg to 1,000 mg, 150 mg to 1,050 mg, 150 mg to 1,100 mg, 150 mg to 1,150 mg, 150 mg to 1,200 mg, 150 mg to 1,250 mg, or 150 mg to 1,300 mg of a targeting moiety per gram of a nucleic acid encoding a cargo; 200 mg to 250 mg, 200 mg to 300 mg, 200 mg to 350 mg, 200 mg to 400 mg, 200 mg to 450 mg, 200 mg to 500 mg, 200 mg to 550 mg, 200 mg to 600 mg, 200 mg to 650 mg, 200 mg to 700 mg, 200 mg to 750 mg, 200 mg to 800 mg, 200 mg to 850 mg, 200 mg to 900 mg, 200 mg to 950 mg, 200 mg to 1,000 mg, 200 mg to 1,050 mg, 200 mg to 1,100 mg, 200 mg to 1,150 mg, 200 mg to 1,200 mg, 200 mg to 1,250 mg, or 200 mg to 1,300 mg of a targeting moiety per gram of a nucleic acid encoding a cargo; 250 mg to 300 mg, 250 mg to 350 mg, 250 mg to 400 mg, 250 mg to 450 mg, 250 mg to 500 mg, 250 mg to 550 mg, 250 mg to 600 mg, 250 mg to 650 mg, 250 mg to 700 mg, 250 mg to 750 mg, 250 mg to 800 mg, 250 mg to 850 mg, 250 mg to 900 mg, 250 mg to 950 mg, 250 mg to 1,000 mg, 250 mg to 1,050 mg, 250 mg to 1,100 mg, 250 mg to 1,150 mg, 250 mg to 1,200 mg, or 250 mg to 1,250 mg of a targeting moiety per gram of a nucleic acid encoding a cargo; 300 mg to 350 mg, 300 mg to 400 mg, 300 mg to 450 mg, 300 mg to 500 mg, 300 mg to 550 mg, 300 mg to 600 mg, 300 mg to 650 mg, 300 mg to 700 mg, 300 mg to 750 mg, 300 mg to 800 mg, 300 mg to 850 mg, 300 mg to 900 mg, 300 mg to 950 mg, 300 mg to 1,000 mg, 300 mg to 1,050 mg, 300 mg to 1,100 mg, 300 mg to 1,150 mg, or 300 mg to 1,200 mg of a targeting moiety per gram of a nucleic acid encoding a cargo; 350 mg to 400 mg, 350 mg to 450 mg, 350 mg to 500 mg, 350 mg to 550 mg, 350 mg to 600 mg, 350 mg to 650 mg, 350 mg to 700 mg, 350 mg to 750 mg, 350 mg to 800 mg, 350 mg to 850 mg, 350 mg to 900 mg, 350 mg to 950 mg, 350 mg to 1,000 mg, 350 mg to 1,050 mg, 350 mg to 1,100 mg, or 350 mg to 1,150 mg of a targeting moiety per gram of a nucleic acid encoding a cargo; 400 mg to 450 mg, 400 mg to 500 mg, 400 mg to 550 mg, 400 mg to 600 mg, 400 mg to 650 mg, 400 mg to 700 mg, 400 mg to 750 mg, 400 mg to 800 mg, 400 mg to 850 mg, 400 mg to 900 mg, 400 mg to 950 mg, 400 mg to 1,000 mg, 400 mg to 1,050 mg, or 400 mg to 1,100 mg of a targeting moiety per gram of a nucleic acid encoding a cargo; 450 mg to 500 mg, 450 mg to 550 mg, 450 mg to 600 mg, 450 mg to 650 mg, 450 mg to 700 mg, 450 mg to 750 mg, 450 mg to 800 mg, 450 mg to 850 mg, 450 mg to 900 mg, 450 mg to 950 mg, 450 mg to 1,000 mg, or 450 mg to 1,050 mg of a targeting moiety per gram of a nucleic acid encoding a cargo; 500 mg to 550 mg, 500 mg to 600 mg, 500 mg to 650 mg, 500 mg to 700 mg, 500 mg to 750 mg, 500 mg to 800 mg, 500 mg to 850 mg, 500 mg to 900 mg, 500 mg to 950 mg, or 500 mg to 1,000 mg of a targeting moiety per gram of a nucleic acid encoding a cargo; 550 mg to 600 mg, 550 mg to 650 mg, 550 mg to 700 mg, 550 mg to 750 mg, 550 mg to 800 mg, 550 mg to 850 mg, 550 mg to 900 mg, or 550 mg to 950 mg of a targeting moiety per gram of a nucleic acid encoding a cargo; 600 mg to 650 mg, 600 mg to 700 mg, 600 mg to 750 mg, 600 mg to 800 mg, 600 mg to 850 mg, 600 mg to 900 mg, or 600 mg to 950 mg of a targeting moiety per gram of a nucleic acid encoding a cargo; 650 mg to 700 mg, 650 mg to 750 mg, 650 mg to 800 mg, 650 mg to 850 mg, 650 mg to 900 mg, or 650 mg to 950 mg of a targeting moiety per gram of a nucleic acid encoding a cargo; 700 mg to 750 mg, 700 mg to 800 mg, 700 mg to 850 mg, 700 mg to 900 mg, or 700 mg to 950 mg of a targeting moiety per gram of a nucleic acid encoding a cargo; 750 mg to 800 mg, 750 mg to 850 mg, 750 mg to 900 mg, or 750 mg to 950 mg of a targeting moiety per gram of a nucleic acid encoding a cargo; 800 mg to 850 mg, 800 mg to 900 mg, 800 mg to 950 mg, 800 mg to 1,000 mg, 800 mg to 1,050 mg, 800 mg to 1,100 mg, 800 mg to 1,150 mg, or 800 mg to 1,200 mg of a targeting moiety per gram of a nucleic acid encoding a cargo; 850 mg to 900 mg, 850 mg to 950 mg, 850 mg to 1,000 mg, 850 mg to 1,050 mg, 850 mg to 1,100 mg, or 850 mg to 1,150 mg of a targeting moiety per gram of a nucleic acid encoding a cargo; 900 mg to 950 mg, 900 mg to 1,000 mg, 900 mg to 1,050 mg, 900 mg to 1,100 mg, or 900 mg to 1,150 mg of a targeting moiety per gram of a nucleic acid encoding a cargo; 950 mg to 1,000 mg, 950 mg to 1,050 mg, 950 mg to 1,100 mg, or 950 mg to 1,150 mg of a targeting moiety per gram of a nucleic acid encoding a cargo; 1,000 mg to 1,050 mg, 1,000 mg to 1,100 mg, or 1,000 mg to 1,150 mg of a targeting moiety per gram of a nucleic acid encoding a cargo; 1,050 mg to 1,100 mg, or 1,050 mg to 1,150 mg of a targeting moiety per gram of a nucleic acid encoding a cargo; 1,100 mg to 1,150 mg, 1,100 mg to 1,200 mg, 1,100 mg to 1,250 mg, 1,100 mg to 1,300 mg, 1,100 mg to 1,350 mg, or 1,100 mg to 1,400 mg of a targeting moiety per gram of a nucleic acid encoding a cargo; 1,150 mg to 1,200 mg, 1,150 mg to 1,250 mg, 1,150 mg to 1,300 mg, 1,150 mg to 1,350 mg, or 1,150 mg to 1,400 mg of a targeting moiety per gram of a nucleic acid encoding a cargo; 1,200 mg to 1,250 mg, 1,200 mg to 1,300 mg, 1,200 mg to 1,350 mg, or 1,200 mg to 1,400 mg of a targeting moiety per gram of a nucleic acid encoding a cargo; 1,250 mg to 1,300 mg, 1,250 mg to 1,350 mg, or 1,250 mg to 1,400 mg of a targeting moiety per gram of a nucleic acid encoding a cargo; 1,300 mg to 1,350 mg, or 1,300 mg to 1,400 mg of a targeting moiety per gram of a nucleic acid encoding a cargo; or 1,350 mg to 1,400 mg of a targeting moiety per gram of a nucleic acid encoding a cargo. In certain embodiments, the amount of the targeting moiety is from 800 mg to 1,200 mg per gram of the nucleic acid cargo. In some embodiments, the amount of the targeting moiety is from 0.5×, 0.6×, 0.7×, 0.8×, 0.9×, 1.0×, 1.1×, 1.2×, 1.3×, 1.4×, 1.5×, 1.6×, 1.7×, 1.8× the amount of nucleic acid cargo.

In some embodiments, the process produces a targeted nanoparticle comprising from 0.15 μg to 25 mg, from 0.15 μg to 20 mg, from 0.15 μg to 15 mg, from 0.15 μg to 10 mg, from 0.15 μg to 5 mg, from 0.15 μg to 1 mg of the targeted moiety incorporated into its outer membrane.

### III. Processes for Manufacturing a Targeted Lipid Nanoparticle

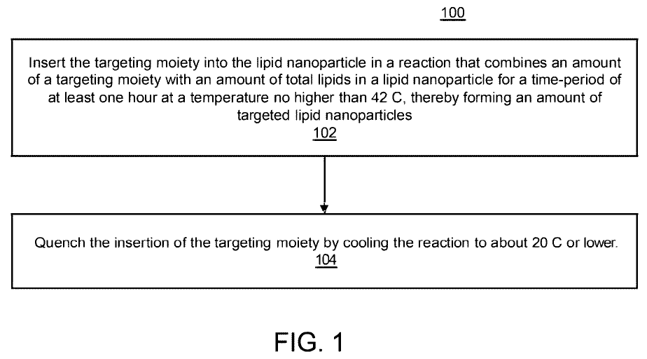

FIG. 1 is a schematic of various steps in a process 100 for insertion of a targeting moiety into a lipid nanoparticle that have been methodically modified and improved as described herein. In the example shown, the targeting moiety is inserted into the lipid nanoparticle at 110. In some embodiments, the targeting moiety is inserted into the lipid nanoparticle utilizing a reaction that combines suitable amounts of the targeting moiety and suitable amounts of the lipid nanoparticle. In many cases, the reaction can combine at least 0.15 μg of the targeting moiety per gram of the total lipids in the lipid nanoparticle. For instance, a soluble reaction can combine from 0.15 μg of the targeting moiety per gram of the total lipid nanoparticle to 30 g of the targeting moiety per gram of the total lipid nanoparticle for a time-period of at least one hour at in a lipid nanoparticle having a nucleic acid cargo encapsulated therein.

In some embodiments that can be combined with any of the preceding embodiments, an amount of the targeting moiety is used in the reaction. In some embodiments that can be combined with any of the preceding embodiments, the amount of the targeting moiety is at least about 10 milligrams of targeting moiety per mole of total lipids in the lipid nanoparticle (mg/mol), 20 mg/mol, 30 mg/mol, 40 mg/mol, 50 mg/mol, 60 mg/mol, 70 mg/mol, 80 mg/mol, 90 mg/mol, 100 mg/mol, 200 mg/mol, 300 mg/mol, 400 mg/mol, 500 mg/mol, 600 mg/mol, 700 mg/mol, 800 mg/mol, 900 mg/mol, 1 gram of targeting moiety per mole of total lipids in the lipid nanoparticle (g/mol), 2 g/mol, 3 g/mol, 4 g/mol, 5 g/mol, 6 g/mol, 7 g/mol, 8 g/mol, 9 g/mol, 10 g/mol, 20 g/mol, 30 g/mol, 40 g/mol, 50 g/mol, or more, at most about 50 g/mol, 40 g/mol, 30 g/mol, 20 g/mol, 10 g/mol, 9 g/mol, 8 g/mol, 7 g/mol, 6 g/mol, 5 g/mol, 4 g/mol, 3 g/mol, 2 g/mol, 1 g/mol, 900 mg/mol, 800 mg/mol, 700 mg/mol, 600 mg/mol, 500 mg/mol, 400 mg/mol, 300 mg/mol, 200 mg/mol, 100 mg/mol, 90 mg/mol, 80 mg/mol, 70 mg/mol, 60 mg/mol, 50 mg/mol, 40 mg/mol, 30 mg/mol, 20 mg/mol, 10 mg/mol, or less, or within a range defined by any two of the preceding values. For instance, in some embodiments, the amount of the targeting moiety is between about 10 mg/mol and about 20 mg/mol, between about 10 mg/mol and about 50 mg/mol, between about 10 mg/mol and about 100 mg/mol, between about 10 mg/mol and about 200 mg/mol, between about 10 mg/mol and about 500 mg/mol, between about 10 mg/mol and about 1 g/mol, between about 10 mg/mol and about 2 g/mol, between about 10 mg/mol and about 5 g/mol, between about 10 mg/mol and about 10 g/mol, between about mg/mol and about 20 g/mol, between about 10 mg/mol and about 50 g/mol, between about 20 mg/mol and about 50 mg/mol, between about 20 mg/mol and about 100 mg/mol, between about 20 mg/mol and about 200 mg/mol, between about 20 mg/mol and about 500 mg/mol, between about 20 mg/mol and about 1 g/mol, between about 20 mg/mol and about 2 g/mol, between about 20 mg/mol and about 5 g/mol, between about 20 mg/mol and about 10 g/mol, between about 20 mg/mol and about 20 g/mol, between about 20 mg/mol and about 50 g/mol, between about 50 mg/mol and about 100 mg/mol, between about 50 mg/mol and about 200 mg/mol, between about 50 mg/mol and about 500 mg/mol, between about 50 mg/mol and about 1 g/mol, between about 50 mg/mol and about 2 g/mol, between about 50 mg/mol and about 5 g/mol, between about 50 mg/mol and about 10 g/mol, between about 50 mg/mol and about 20 g/mol, between about 50 mg/mol and about 50 g/mol, between about 100 mg/mol and about 200 mg/mol, between about 100 mg/mol and about 500 mg/mol, between about 100 mg/mol and about 1 g/mol, between about 100 mg/mol and about 2 g/mol, between about 100 mg/mol and about 5 g/mol, between about 100 mg/mol and about 10 g/mol, between about 100 mg/mol and about 20 g/mol, between about 100 mg/mol and about 50 g/mol, between about 200 mg/mol and about 500 mg/mol, between about 200 mg/mol and about 1 g/mol, between about 200 mg/mol and about 2 g/mol, between about 200 mg/mol and about 5 g/mol, between about 200 mg/mol and about 10 g/mol, between about 200 mg/mol and about 20 g/mol, between about 200 mg/mol and about 50 g/mol, between about 500 mg/mol and about 1 g/mol, between about 500 mg/mol and about 2 g/mol, between about 500 mg/mol and about 5 g/mol, between about 500 mg/mol and about 10 g/mol, between about 500 mg/mol and about 20 g/mol, between about 500 mg/mol and about 50 g/mol, between about 1 g/mol and about 2 g/mol, between about 1 g/mol and about 5 g/mol, between about 1 g/mol and about 10 g/mol, between about 1 g/mol and about 20 g/mol, between about 1 g/mol and about 50 g/mol, between about 2 g/mol and about 5 g/mol, between about 2 g/mol and about 10 g/mol, between about 2 g/mol and about 20 g/mol, between about 2 g/mol and about 50 g/mol, between about 5 g/mol and about 10 g/mol, between about 5 g/mol and about 20 g/mol, between about 5 g/mol and about 50 g/mol, between about 10 g/mol and about 20 g/mol, between about 10 g/mol and about 50 g/mol, or between about 10 g/mol and about 50 g/mol.

In some embodiments that can be combined with any of the preceding embodiments, the reaction or the insertion of the targeting moiety into the lipid nanoparticles is carried out for a period of time (also referred to herein as a “time period” or “time-period”). In some embodiments, the period of time is at least about 30 minutes (min), 40 min, 50 min, 1 hour (h), 2 h, 3 h, 4 h, 5 h, 6 h, 7 h, 8 h, or more, at most about 8 h, 7 h, 6 h, 5 h, 4 h, 3 h, 2 h, 1 h, 50 min, 40 min, 30 min, or less, or a period of time that is within a range defined by any two of the preceding values. For instance, in some embodiments, the period of time is between about 30 min and about 8 h, between about 30 min and about 6 h, between about 30 min and about 4 h, between about 30 min and about 2 h, between about 30 min and about 1 h, between about 1 h and about 8 h, between about 1 h and about 6 h, between about 1 h and about 4 h, between about 1 h and about 2 h, between about 2 h and about 8 h, between about 2 h and about 6 h, between about 2 h and about 4 h, or between about 4 h and about 8 h.

In some embodiments that can be combined with any of the preceding embodiments, the reaction or the insertion of the targeting moiety into the lipid nanoparticles is carried out at a temperature. In some embodiments, the temperature is at least about 20 degrees Centigrade (° C.), 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., or more, at most about 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., or less, or within a range defined by any two of the preceding values. For instances, in some embodiments, the temperature is between about 20° C. and about 60° C., between about 20° C. and about 58° C., between about 20° C. and about 56° C., between about 20° C. and about 54° C., between about 20° C. and about 52° C., between about 20° C. and about 50° C., between about 20° C. and about 48° C., between about 20° C. and about 46° C., between about 20° C. and about 44° C., between about 20° C. and about 42° C., between about 20° C. and about 40° C., between about 20° C. and about 38° C., between about 20° C. and about 36° C., between about 20° C. and about 34° C., between about 20° C. and about 32° C., between about 20° C. and about 30° C., between about 20° C. and about 28° C., between about 20° C. and about 26° C., between about 20° C. and about 24° C., between about 20° C. and about 22° C., between about 22° C. and about 60° C., between about 22° C. and about 58° C., between about 22° C. and about 56° C., between about 22° C. and about 54° C., between about 22° C. and about 52° C., between about 22° C. and about 50° C., between about 22° C. and about 48° C., between about 22° C. and about 46° C., between about 22° C. and about 44° C., between about 22° C. and about 42° C., between about 22° C. and about 40° C., between about 22° C. and about 38° C., between about 22° C. and about 36° C., between about 22° C. and about 34° C., between about 22° C. and about 32° C., between about 22° C. and about 30° C., between about 22° C. and about 28° C., between about 22° C. and about 26° C., between about 22° C. and about 24° C., between about 24° C. and about 60° C., between about 24° C. and about 58° C., between about 24° C. and about 56° C., between about 24° C. and about 54° C., between about 24° C. and about 52° C., between about 24° C. and about 50° C., between about 24° C. and about 48° C., between about 24° C. and about 46° C., between about 24° C. and about 44° C., between about 24° C. and about 42° C., between about 24° C. and about 40° C., between about 24° C. and about 38° C., between about 24° C. and about 36° C., between about 24° C. and about 34° C., between about 24° C. and about 32° C., between about 24° C. and about 30° C., between about 24° C. and about 28° C., between about 24° C. and about 26° C., between about 26° C. and about 60° C., between about 26° C. and about 58° C., between about 26° C. and about 56° C., between about 26° C. and about 54° C., between about 26° C. and about 52° C., between about 26° C. and about 50° C., between about 26° C. and about 48° C., between about 26° C. and about 46° C., between about 26° C. and about 44° C., between about 26° C. and about 42° C., between about 26° C. and about 40° C., between about 26° C. and about 38° C., between about 26° C. and about 36° C., between about 26° C. and about 34° C., between about 26° C. and about 32° C., between about 26° C. and about 30° C., between about 26° C. and about 28° C., between about 28° C. and about 60° C., between about 28° C. and about 58° C., between about 28° C. and about 56° C., between about 28° C. and about 54° C., between about 28° C. and about 52° C., between about 28° C. and about 50° C., between about 28° C. and about 48° C., between about 28° C. and about 46° C., between about 28° C. and about 44° C., between about 28° C. and about 42° C., between about 28° C. and about 40° C., between about 28° C. and about 38° C., between about 28° C. and about 36° C., between about 28° C. and about 34° C., between about 28° C. and about 34° C., between about 28° C. and about 32° C., between about 28° C. and about 30° C., between about 30° C. and about 60° C., between about 30° C. and about 58° C., between about 30° C. and about 56° C., between about 30° C. and about 54° C., between about 30° C. and about 52° C., between about 30° C. and about 50° C., between about 30° C. and about 48° C., between about 30° C. and about 46° C., between about 30° C. and about 44° C., between about 30° C. and about 42° C., between about 30° C. and about 40° C., between about 30° C. and about 38° C., between about 30° C. and about 36° C., between about 30° C. and about 34° C., between about 30° C. and about 32° C., between about 32° C. and about 60° C., between about 32° C. and about 58° C., between about 32° C. and about 56° C., between about 32° C. and about 54° C., between about 32° C. and about 52° C., between about 32° C. and about 50° C., between about 32° C. and about 48° C., between about 32° C. and about 46° C., between about 32° C. and about 44° C., between about 32° C. and about 42° C., between about 32° C. and about 40° C., between about 32° C. and about 38° C., between about 32° C. and about 36° C., between about 32° C. and about 34° C., between about 34° C. and about 60° C., between about 34° C. and about 58° C., between about 34° C. and about 56° C., between about 34° C. and about 54° C., between about 34° C. and about 52° C., between about 34° C. and about 50° C., between about 34° C. and about 48° C., between about 34° C. and about 46° C., between about 34° C. and about 44° C., between about 34° C. and about 42° C., between about 34° C. and about 40° C., between about 34° C. and about 38° C., between about 34° C. and about 36° C., between about 36° C. and about 60° C., between about 36° C. and about 58° C., between about 36° C. and about 56° C., between about 36° C. and about 54° C., between about 36° C. and about 52° C., between about 36° C. and about 50° C., between about 36° C. and about 48° C., between about 36° C. and about 46° C., between about 36° C. and about 44° C., between about 36° C. and about 42° C., between about 36° C. and about 40° C., between about 36° C. and about 38° C., between about 38° C. and about 60° C., between about 38° C. and about 58° C., between about 38° C. and about 56° C., between about 38° C. and about 54° C., between about 38° C. and about 52° C., between about 38° C. and about 50° C., between about 38° C. and about 48° C., between about 38° C. and about 46° C., between about 38° C. and about 44° C., between about 38° C. and about 42° C., between about 38° C. and about 40° C., between about 40° C. and about 60° C., between about 40° C. and about 58° C., between about 40° C. and about 56° C., between about 40° C. and about 54° C., between about 40° C. and about 52° C., between about 40° C. and about 50° C., between about 40° C. and about 48° C., between about 40° C. and about 46° C., between about 40° C. and about 44° C., between about 40° C. and about 42° C., between about 42° C. and about 60° C., between about 42° C. and about 58° C., between about 42° C. and about 56° C., between about 42° C. and about 54° C., between about 42° C. and about 52° C., between about 42° C. and about 50° C., between about 42° C. and about 48° C., between about 42° C. and about 46° C., between about 42° C. and about 44° C., between about 44° C. and about 60° C., between about 44° C. and about 58° C., between about 44° C. and about 56° C., between about 44° C. and about 54° C., between about 44° C. and about 52° C., between about 44° C. and about 50° C., between about 44° C. and about 48° C., between about 44° C. and about 46° C., between about 46° C. and about 60° C., between about 46° C. and about 58° C., between about 46° C. and about 56° C., between about 46° C. and about 54° C., between about 46° C. and about 52° C., between about 46° C. and about 50° C., between about 46° C. and about 48° C., between about 48° C. and about 60° C., between about 48° C. and about 58° C., between about 48° C. and about 56° C., between about 48° C. and about 54° C., between about 48° C. and about 52° C., between about 48° C. and about 50° C., between about 50° C. and about 60° C., between about 50° C. and about 58° C., between about 50° C. and about 56° C., between about 50° C. and about 54° C., between about 50° C. and about 52° C., between about 52° C. and about 60° C., between about 52° C. and about 58° C., between about 52° C. and about 56° C., between about 52° C. and about 54° C., between about 54° C. and about 60° C., between about 54° C. and about 58° C., between about 54° C. and about 56° C., between about 56° C. and about 60° C., between about 56° C. and about 58° C., or between about 58° C. and about 60° C.

In some embodiments that can be combined with any of the preceding embodiments, the reaction or the insertion of the targeting moiety into the lipid nanoparticles is conducted at a pH. In some embodiments, the pH is at least about 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, or more, at most about 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, or less, or within a range defined by any two of the preceding values. For instance, in some embodiments, the pH is between about 6 and about 6.1, between about 6 and about 6.2, between about 6 and about 6.3, between about 6 and about 6.4, between about 6 and about 6.5, between about 6 and about 6.6, between about 6 and about 6.7, between about 6 and about 6.8, between about 6 and about 6.9, between about 6 and about 7, between about 6 and about 7.1, between about 6 and about 7.2, between about 6 and about 7.3, between about 6 and about 7.4, between about 6 and about 7.5, between about 6 and about 7.6, between about 6 and about 7.7, between about 6 and about 7.8, between about 6 and about 7.9, between about 6 and about 8, between about 6.1 and about 6.2, between about 6.1 and about 6.3, between about 6.1 and about 6.4, between about 6.1 and about 6.5, between about 6.1 and about 6.6, between about 6.1 and about 6.7, between about 6.1 and about 6.8, between about 6.1 and about 6.9, between about 6.1 and about 7, between about 6.1 and about 7.1, between about 6.1 and about 7.2, between about 6.1 and about 7.3, between about 6.1 and about 7.4, between about 6.1 and about 7.5, between about 6.1 and about 7.6, between about 6.1 and about 7.7, between about 6.1 and about 7.8, between about 6.1 and about 7.9, between about 6.1 and about 8, between about 6.2 and about 6.3, between about 6.2 and about 6.4, between about 6.2 and about 6.5, between about 6.2 and about 6.6, between about 6.2 and about 6.7, between about 6.2 and about 6.8, between about 6.2 and about 6.9, between about 6.2 and about 7, between about 6.2 and about 7.1, between about 6.2 and about 7.2, between about 6.2 and about 7.3, between about 6.2 and about 7.4, between about 6.2 and about 7.5, between about 6.2 and about 7.6, between about 6.2 and about 7.7, between about 6.2 and about 7.8, between about 6.2 and about 7.9, between about 6.2 and about 8, between about 6.3 and about 6.4, between about 6.3 and about 6.5, between about 6.3 and about 6.6, between about 6.3 and about 6.7, between about 6.3 and about 6.8, between about 6.3 and about 6.9, between about 6.3 and about 7, between about 6.3 and about 7.1, between about 6.3 and about 7.2, between about 6.3 and about 7.3, between about 6.3 and about 7.4, between about 6.3 and about 7.5, between about 6.3 and about 7.6, between about 6.3 and about 7.7, between about 6.3 and about 7.8, between about 6.3 and about 7.9, between about 6.3 and about 8, between about 6.4 and about 6.5, between about 6.4 and about 6.6, between about 6.4 and about 6.7, between about 6.4 and about 6.8, between about 6.4 and about 6.9, between about 6.4 and about 7, between about 6.4 and about 7.1, between about 6.4 and about 7.2, between about 6.4 and about 7.3, between about 6.4 and about 7.4, between about 6.4 and about 7.5, between about 6.4 and about 7.6, between about 6.4 and about 7.7, between about 6.4 and about 7.8, between about 6.4 and about 7.9, between about 6.4 and about 8, between about 6.5 and about 6.6, between about 6.5 and about 6.7, between about 6.5 and about 6.8, between about 6.5 and about 6.9, between about 6.5 and about 7, between about 6.5 and about 7.1, between about 6.5 and about 7.2, between about 6.5 and about 7.3, between about 6.5 and about 7.4, between about 6.5 and about 7.5, between about 6.5 and about 7.6, between about 6.5 and about 7.7, between about 6.5 and about 7.8, between about 6.5 and about 7.9, between about 6.5 and about 8, between about 6.6 and about 6.7, between about 6.6 and about 6.8, between about 6.6 and about 6.9, between about 6.6 and about 7, between about 6.6 and about 7.1, between about 6.6 and about 7.2, between about 6.6 and about 7.3, between about 6.6 and about 7.4, between about 6.6 and about 7.5, between about 6.6 and about 7.6, between about 6.6 and about 7.7, between about 6.6 and about 7.8, between about 6.6 and about 7.9, between about 6.6 and about 8, between about 6.7 and about 6.8, between about 6.7 and about 6.9, between about 6.7 and about 7, between about 6.7 and about 7.1, between about 6.7 and about 7.2, between about 6.7 and about 7.3, between about 6.7 and about 7.4, between about 6.7 and about 7.5, between about 6.7 and about 7.6, between about 6.7 and about 7.7, between about 6.7 and about 7.8, between about 6.7 and about 7.9, between about 6.7 and about 8, between about 6.8 and about 6.9, between about 6.8 and about 7, between about 6.8 and about 7.1, between about 6.8 and about 7.2, between about 6.8 and about 7.3, between about 6.8 and about 7.4, between about 6.8 and about 7.5, between about 6.8 and about 7.6, between about 6.8 and about 7.7, between about 6.8 and about 7.8, between about 6.8 and about 7.9, between about 6.8 and about 8, between about 6.9 and about 7, between about 6.9 and about 7.1, between about 6.9 and about 7.2, between about 6.9 and about 7.3, between about 6.9 and about 7.4, between about 6.9 and about 7.5, between about 6.9 and about 7.6, between about 6.9 and about 7.7, between about 6.9 and about 7.8, between about 6.9 and about 7.9, between about 6.9 and about 8, between about 7 and about 7.1, between about 7 and about 7.2, between about 7 and about 7.3, between about 7 and about 7.4, between about 7 and about 7.5, between about 7 and about 7.6, between about 7 and about 7.7, between about 7 and about 7.8, between about 7 and about 7.9, between about 7 and about 8, between about 7.1 and about 7.2, between about 7.1 and about 7.3, between about 7.1 and about 7.4, between about 7.1 and about 7.5, between about 7.1 and about 7.6, between about 7.1 and about 7.7, between about 7.1 and about 7.8, between about 7.1 and about 7.9, between about 7.1 and about 8, between about 7.2 and about 7.3, between about 7.2 and about 7.4, between about 7.2 and about 7.5, between about 7.2 and about 7.6, between about 7.2 and about 7.7, between about 7.2 and about 7.8, between about 7.2 and about 7.9, between about 7.2 and about 8, between about 7.3 and about 7.4, between about 7.3 and about 7.5, between about 7.3 and about 7.6, between about 7.3 and about 7.7, between about 7.3 and about 7.8, between about 7.3 and about 7.9, between about 7.3 and about 8, between about 7.4 and about 7.5, between about 7.4 and about 7.6, between about 7.4 and about 7.7, between about 7.4 and about 7.8, between about 7.4 and about 7.9, between about 7.4 and about 8, between about 7.5 and about 7.6, between about 7.5 and about 7.7, between about 7.5 and about 7.8, between about 7.5 and about 7.9, between about 7.5 and about 8, between about 7.6 and about 7.7, between about 7.6 and about 7.8, between about 7.6 and about 7.9, between about 7.6 and about 8, between about 7.7 and about 7.8, between about 7.7 and about 7.9, between about 7.7 and about 8, between about 7.8 and about 7.9, between about 7.8 and about 8, or between about 7.9 and about 8.

In some embodiments that can be combined with any of the preceding embodiments, the reaction or the insertion of the targeting moiety into the lipid nanoparticles is performed in a buffer. In some embodiments, the buffer is 2-(N-morpholino)ethanesulfonic acid (MES) buffer. In some embodiments, the MES buffer is a 25 mM MES buffer. In some embodiments, the MES buffer comprises 150 mM sodium chloride (NaCl) at pH 6.5. In some embodiments, the buffer is tris(hydroxymethyl)aminomethane (Tris) buffer. In some embodiments, the Tris buffer is a 50 mM Tris buffer. In some embodiments, the Tris buffer comprises 150 mM NaCl at pH 7.5. In some embodiments, the buffer is 4-(2-hydroxyethyl)-1-pieerazineethanesulfonic acid (HEPES) buffer. In some embodiments, the HEPES buffer is a 25 mM HEPES buffer. In some embodiments, the HEPES buffer comprises 150 mM NaCl at pH 7.4.

In some embodiments that can be combined with any of the preceding embodiments, the targeting moiety has a molar mass. In some embodiments, the molar mass is at least about 5 kilodaltons (kDa), 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 105 kDa, 115 kDa, 120 kDa, 125 kDa, 130 kDa, 135 kDa, 140 kDa, 145 kDa, 150 kDa, 155 kDa, 160 kDa, 165 kDa, 170 kDa, 175 kDa, 180 kDa, 185 kDa, 190 kDa, 195 kDa, 200 kDa, 205 kDa, 210 kDa, 215 kDa, 220 kDa, 225 kDa, 230 kDa, 235 kDa, 240 kDa, 245 kDa, 250 kDa, or more, 250 kDa, 245 kDa, 240 kDa, 235 kDa, 230 kDa, 225 kDa, 220 kDa, 215 kDa, 210 kDa, 205 kDa, 200 kDa, 195 kDa, 190 kDa, 185 kDa, 180 kDa, 175 kDa, 170 kDa, 165 kDa, 160 kDa, 155 kDa, 150 kDa, 145 kDa, 140 kDa, 135 kDa, 130 kDa, 125 kDa, 120 kDa, 115 kDa, 110 kDa, 105 kDa, 100 kDa, 95 kDa, 90 kDa, 85 kDa, 80 kDa, 75 kDa, 70 kDa, 65 kDa, 60 kDa, 55 kDa, 50 kDa, 45 kDa, 40 kDa, 35 kDa, 30 kDa, 25 kDa, 20 kDa, 15 kDa, 10 kDa, 5 kDa, or less, or within a range defined by any two of the preceding values.

In some embodiments that may be combined with preceding embodiments, the targeting moiety comprises an antigen-binding protein. In some embodiments, the antigen binding protein is encapsulated in a micelle prior to addition to the reaction. In some embodiments, the micelle encapsulated antigen-binding protein is an antigen-binding protein polyethylene glycol-micelle (antigen-binding protein-PEG-micelle) moiety. In some embodiments, the antigen-binding protein-PEG-micelle moiety comprises a heat-shock-protein 34 antigen-binding protein-PEG micelle moiety. In some embodiments, the targeting moiety comprises a fragment antigen-binding (Fab) moiety.

In some embodiments that may be combined with preceding embodiments, the nucleic acid cargo is an mRNA encoding a chimeric antigen receptor (CAR) moiety. In some embodiments that can be combined with preceding embodiments, the insertion of the targeting moiety into the lipid nanoparticle comprises combining a certain amount of the targeting moiety with a certain amount of lipid nanoparticles having a certain amount of the nucleic acid cargo therein. In some embodiments, the amount of the targeting moiety to nucleic acid cargo is from 15 mg of targeting moiety per gram of nucleic acid in the lipid nanoparticle, from 16 mg of targeting moiety per gram of nucleic acid in the lipid nanoparticle, 17 mg of targeting moiety per gram of nucleic acid in the lipid nanoparticle, 18 mg of targeting moiety per gram of nucleic acid in the lipid nanoparticle, 19 mg of targeting moiety per gram of nucleic acid in the lipid nanoparticle, 20 mg of targeting moiety per gram of nucleic acid in the lipid nanoparticle, 21 mg of targeting moiety per gram of nucleic acid in the lipid nanoparticle, 22 mg of targeting moiety per gram of nucleic acid in the lipid nanoparticle, 23 mg of targeting moiety per gram of nucleic acid in the lipid nanoparticle, 24 mg of targeting moiety per gram of nucleic acid in the lipid nanoparticle, 25 mg of targeting moiety per gram of nucleic acid in the lipid nanoparticle, 26 mg of targeting moiety per gram of nucleic acid in the lipid nanoparticle, 27 mg of targeting moiety per gram of nucleic acid in the lipid nanoparticle, 28 mg of targeting moiety per gram of nucleic acid in the lipid nanoparticle, 29 mg of targeting moiety per gram of nucleic acid in the lipid nanoparticle, 30 mg of targeting moiety per gram of nucleic acid in the lipid nanoparticle, 31 mg of targeting moiety per gram of nucleic acid in the lipid nanoparticle, 32 mg of targeting moiety per gram of nucleic acid in the lipid nanoparticle, 33 mg of targeting moiety per gram of nucleic acid in the lipid nanoparticle, 34 mg of targeting moiety per gram of nucleic acid in the lipid nanoparticle, 35 mg of targeting moiety per gram of nucleic acid in the lipid nanoparticle, 36 mg of targeting moiety per gram of nucleic acid in the lipid nanoparticle, 37 mg of targeting moiety per gram of nucleic acid in the lipid nanoparticle, 38 mg of targeting moiety per gram of nucleic acid in the lipid nanoparticle, 39 mg of targeting moiety per gram of nucleic acid in the lipid nanoparticle, 40 mg of targeting moiety per gram of nucleic acid in the lipid nanoparticle, 41 mg of targeting moiety per gram of nucleic acid in the lipid nanoparticle, 42 mg of targeting moiety per gram of nucleic acid in the lipid nanoparticle, 43 mg of targeting moiety per gram of nucleic acid in the lipid nanoparticle, 44 mg of targeting moiety per gram of nucleic acid in the lipid nanoparticle, 45 mg of targeting moiety per gram of nucleic acid in the lipid nanoparticle, 46 mg of targeting moiety per gram of nucleic acid in the lipid nanoparticle, 47 mg of targeting moiety per gram of nucleic acid in the lipid nanoparticle, 48 mg of targeting moiety per gram of nucleic acid in the lipid nanoparticle, 49 mg of targeting moiety per gram of nucleic acid in the lipid nanoparticle, 50 mg of targeting moiety per gram of nucleic acid in the lipid nanoparticle, 51 mg of targeting moiety per gram of nucleic acid in the lipid nanoparticle, 52 mg of targeting moiety per gram of nucleic acid in the lipid nanoparticle, 53 mg of targeting moiety per gram of nucleic acid in the lipid nanoparticle, 54 mg of targeting moiety per gram of nucleic acid in the lipid nanoparticle, 55 mg of targeting moiety per gram of nucleic acid in the lipid nanoparticle, 56 mg of targeting moiety per gram of nucleic acid in the lipid nanoparticle, 57 mg of targeting moiety per gram of nucleic acid in the lipid nanoparticle, 58 mg of targeting moiety per gram of nucleic acid in the lipid nanoparticle, 59 mg of targeting moiety per gram of nucleic acid in the lipid nanoparticle, 60 mg of targeting moiety per gram of nucleic acid in the lipid nanoparticle, 61 mg of targeting moiety per gram of nucleic acid in the lipid nanoparticle, 62 mg of targeting moiety per gram of nucleic acid in the lipid nanoparticle, 63 mg of targeting moiety per gram of nucleic acid in the lipid nanoparticle, 64 mg of targeting moiety per gram of nucleic acid in the lipid nanoparticle, 65 mg of targeting moiety per gram of nucleic acid in the lipid nanoparticle, 66 mg of targeting moiety per gram of nucleic acid in the lipid nanoparticle, 67 mg of targeting moiety per gram of nucleic acid in the lipid nanoparticle, 68 mg of targeting moiety per gram of nucleic acid in the lipid nanoparticle, 69 mg of targeting moiety per gram of nucleic acid in the lipid nanoparticle, 70 mg of targeting moiety per gram of nucleic acid in the lipid nanoparticle, 71 mg of targeting moiety per gram of nucleic acid in the lipid nanoparticle, 72 mg of targeting moiety per gram of nucleic acid in the lipid nanoparticle, 73 mg of targeting moiety per gram of nucleic acid in the lipid nanoparticle, 74 mg of targeting moiety per gram of nucleic acid in the lipid nanoparticle, 75 mg of targeting moiety per gram of nucleic acid in the lipid nanoparticle, 76 mg of targeting moiety per gram of nucleic acid in the lipid nanoparticle, 77 mg of targeting moiety per gram of nucleic acid in the lipid nanoparticle, 78 mg of targeting moiety per gram of nucleic acid in the lipid nanoparticle, 79 mg of targeting moiety per gram of nucleic acid in the lipid nanoparticle, mg of targeting moiety per gram of nucleic acid in the lipid nanoparticle, 80 mg of targeting moiety per gram of nucleic acid in the lipid nanoparticle, 81 mg of targeting moiety per gram of nucleic acid in the lipid nanoparticle, 82 mg of targeting moiety per gram of nucleic acid in the lipid nanoparticle, 83 mg of targeting moiety per gram of nucleic acid in the lipid nanoparticle, 84 mg of targeting moiety per gram of nucleic acid in the lipid nanoparticle, 85 mg of targeting moiety per gram of nucleic acid in the lipid nanoparticle, 86 mg of targeting moiety per gram of nucleic acid in the lipid nanoparticle, 87 mg of targeting moiety per gram of nucleic acid in the lipid nanoparticle, 88 mg of targeting moiety per gram of nucleic acid in the lipid nanoparticle, 89 mg of targeting moiety per gram of nucleic acid in the lipid nanoparticle, 90 mg of targeting moiety per gram of nucleic acid in the lipid nanoparticle, 91 mg of targeting moiety per gram of nucleic acid in the lipid nanoparticle, 92 mg of targeting moiety per gram of nucleic acid in the lipid nanoparticle, 93 mg of targeting moiety per gram of nucleic acid in the lipid nanoparticle, 94 mg of targeting moiety per gram of nucleic acid in the lipid nanoparticle, 95 mg of targeting moiety per gram of nucleic acid in the lipid nanoparticle, 96 mg of targeting moiety per gram of nucleic acid in the lipid nanoparticle, 97 mg of targeting moiety per gram of nucleic acid in the lipid nanoparticle, 98 mg of targeting moiety per gram of nucleic acid in the lipid nanoparticle, 99 mg of targeting moiety per gram of nucleic acid in the lipid nanoparticle, 100 mg of targeting moiety per gram of nucleic acid in the lipid nanoparticle, mg of targeting moiety per gram of nucleic acid in the lipid nanoparticle, 101 mg of targeting moiety per gram of nucleic acid in the lipid nanoparticle, 102 mg of targeting moiety per gram of nucleic acid in the lipid nanoparticle, 103 mg of targeting moiety per gram of nucleic acid in the lipid nanoparticle, 104 mg of targeting moiety per gram of nucleic acid in the lipid nanoparticle, 105 mg of targeting moiety per gram of nucleic acid in the lipid nanoparticle, 106 mg of targeting moiety per gram of nucleic acid in the lipid nanoparticle, 107 mg of targeting moiety per gram of nucleic acid in the lipid nanoparticle, 108 mg of targeting moiety per gram of nucleic acid in the lipid nanoparticle, 109 mg of targeting moiety per gram of nucleic acid in the lipid nanoparticle, 110 mg of targeting moiety per gram of nucleic acid in the lipid nanoparticle, 111 mg of targeting moiety per gram of nucleic acid in the lipid nanoparticle, 112 mg of targeting moiety per gram of nucleic acid in the lipid nanoparticle, 113 mg of targeting moiety per gram of nucleic acid in the lipid nanoparticle, 114 mg of targeting moiety per gram of nucleic acid in the lipid nanoparticle, 115 mg of targeting moiety per gram of nucleic acid in the lipid nanoparticle, from 116 mg of targeting moiety per gram of nucleic acid in the lipid nanoparticle, 117 mg of targeting moiety per gram of nucleic acid in the lipid nanoparticle, 118 mg of targeting moiety per gram of nucleic acid in the lipid nanoparticle, 119 mg of targeting moiety per gram of nucleic acid in the lipid nanoparticle, 120 mg of targeting moiety per gram of nucleic acid in the lipid nanoparticle, 121 mg of targeting moiety per gram of nucleic acid in the lipid nanoparticle, 122 mg of targeting moiety per gram of nucleic acid in the lipid nanoparticle, 123 mg of targeting moiety per gram of nucleic acid in the lipid nanoparticle, 124 mg of targeting moiety per gram of nucleic acid in the lipid nanoparticle, 125 mg of targeting moiety per gram of nucleic acid in the lipid nanoparticle, 126 mg of targeting moiety per gram of nucleic acid in the lipid nanoparticle, 127 mg of targeting moiety per gram of nucleic acid in the lipid nanoparticle, 128 mg of targeting moiety per gram of nucleic acid in the lipid nanoparticle, 129 mg of targeting moiety per gram of nucleic acid in the lipid nanoparticle, 130 mg of targeting moiety per gram of nucleic acid in the lipid nanoparticle, 131 mg of targeting moiety per gram of nucleic acid in the lipid nanoparticle, 132 mg of targeting moiety per gram of nucleic acid in the lipid nanoparticle, 133 mg of targeting moiety per gram of nucleic acid in the lipid nanoparticle, 134 mg of targeting moiety per gram of nucleic acid in the lipid nanoparticle, 135 mg of targeting moiety per gram of nucleic acid in the lipid nanoparticle, 136 mg of targeting moiety per gram of nucleic acid in the lipid nanoparticle, 137 mg of targeting moiety per gram of nucleic acid in the lipid nanoparticle, 138 mg of targeting moiety per gram of nucleic acid in the lipid nanoparticle, 139 mg of targeting moiety per gram of nucleic acid in the lipid nanoparticle, 140 mg of targeting moiety per gram of nucleic acid in the lipid nanoparticle, 141 mg of targeting moiety per gram of nucleic acid in the lipid nanoparticle, 142 mg of targeting moiety per gram of nucleic acid in the lipid nanoparticle, 143 mg of targeting moiety per gram of nucleic acid in the lipid nanoparticle, 144 mg of targeting moiety per gram of nucleic acid in the lipid nanoparticle, 145 mg of targeting moiety per gram of nucleic acid in the lipid nanoparticle, 146 mg of targeting moiety per gram of nucleic acid in the lipid nanoparticle, 147 mg of targeting moiety per gram of nucleic acid in the lipid nanoparticle, 148 mg of targeting moiety per gram of nucleic acid in the lipid nanoparticle, 149 mg of targeting moiety per gram of nucleic acid in the lipid nanoparticle, 150 mg of targeting moiety per gram of nucleic acid in the lipid nanoparticle, 151 mg of targeting moiety per gram of nucleic acid in the lipid nanoparticle, 152 mg of targeting moiety per gram of nucleic acid in the lipid nanoparticle, 153 mg of targeting moiety per gram of nucleic acid in the lipid nanoparticle, 154 mg of targeting moiety per gram of nucleic acid in the lipid nanoparticle, 155 mg of targeting moiety per gram of nucleic acid in the lipid nanoparticle, 156 mg of targeting moiety per gram of nucleic acid in the lipid nanoparticle, 157 mg of targeting moiety per gram of nucleic acid in the lipid nanoparticle, 158 mg of targeting moiety per gram of nucleic acid in the lipid nanoparticle, 159 mg of targeting moiety per gram of nucleic acid in the lipid nanoparticle, 160 mg of targeting moiety per gram of nucleic acid in the lipid nanoparticle, 161 mg of targeting moiety per gram of nucleic acid in the lipid nanoparticle, 162 mg of targeting moiety per gram of nucleic acid in the lipid nanoparticle, 163 mg of targeting moiety per gram of nucleic acid in the lipid nanoparticle, 164 mg of targeting moiety per gram of nucleic acid in the lipid nanoparticle, 165 mg of targeting moiety per gram of nucleic acid in the lipid nanoparticle, 166 mg of targeting moiety per gram of nucleic acid in the lipid nanoparticle, 167 mg of targeting moiety per gram of nucleic acid in the lipid nanoparticle, 168 mg of targeting moiety per gram of nucleic acid in the lipid nanoparticle, 169 mg of targeting moiety per gram of nucleic acid in the lipid nanoparticle, 170 mg of targeting moiety per gram of nucleic acid in the lipid nanoparticle, 171 mg of targeting moiety per gram of nucleic acid in the lipid nanoparticle, from 172 mg of targeting moiety per gram of nucleic acid in the lipid nanoparticle, 173 mg of targeting moiety per gram of nucleic acid in the lipid nanoparticle, 174 mg of targeting moiety per gram of nucleic acid in the lipid nanoparticle, 175 mg of targeting moiety per gram of nucleic acid in the lipid nanoparticle, 176 mg of targeting moiety per gram of nucleic acid in the lipid nanoparticle, 177 mg of targeting moiety per gram of nucleic acid in the lipid nanoparticle, 178 mg of targeting moiety per gram of nucleic acid in the lipid nanoparticle, 179 mg of targeting moiety per gram of nucleic acid in the lipid nanoparticle, 180 mg of targeting moiety per gram of nucleic acid in the lipid nanoparticle, 181 mg of targeting moiety per gram of nucleic acid in the lipid nanoparticle, 182 mg of targeting moiety per gram of nucleic acid in the lipid nanoparticle, 183 mg of targeting moiety per gram of nucleic acid in the lipid nanoparticle, 184 mg of targeting moiety per gram of nucleic acid in the lipid nanoparticle, 185 mg of targeting moiety per gram of nucleic acid in the lipid nanoparticle, 186 mg of targeting moiety per gram of nucleic acid in the lipid nanoparticle, 187 mg of targeting moiety per gram of nucleic acid in the lipid nanoparticle, 188 mg of targeting moiety per gram of nucleic acid in the lipid nanoparticle, 189 mg of targeting moiety per gram of nucleic acid in the lipid nanoparticle, 190 mg of targeting moiety per gram of nucleic acid in the lipid nanoparticle, 191 mg of targeting moiety per gram of nucleic acid in the lipid nanoparticle, 192 mg of targeting moiety per gram of nucleic acid in the lipid nanoparticle, 193 mg of targeting moiety per gram of nucleic acid in the lipid nanoparticle, 194 mg of targeting moiety per gram of nucleic acid in the lipid nanoparticle, 195 mg of targeting moiety per gram of nucleic acid in the lipid nanoparticle, 196 mg of targeting moiety per gram of nucleic acid in the lipid nanoparticle, 197 mg of targeting moiety per gram of nucleic acid in the lipid nanoparticle, 198 mg of targeting moiety per gram of nucleic acid in the lipid nanoparticle, 199 mg of targeting moiety per gram of nucleic acid in the lipid nanoparticle, 200 mg of targeting moiety per gram of nucleic acid in the lipid nanoparticle, 201 mg of targeting moiety per gram of nucleic acid in the lipid nanoparticle, 202 mg of targeting moiety per gram of nucleic acid in the lipid nanoparticle, 203 mg of targeting moiety per gram of nucleic acid in the lipid nanoparticle, 204 mg of targeting moiety per gram of nucleic acid in the lipid nanoparticle, 205 mg of targeting moiety per gram of nucleic acid in the lipid nanoparticle, 206 mg of targeting moiety per gram of nucleic acid in the lipid nanoparticle, 207 mg of targeting moiety per gram of nucleic acid in the lipid nanoparticle, 208 mg of targeting moiety per gram of nucleic acid in the lipid nanoparticle, 209 mg of targeting moiety per gram of nucleic acid in the lipid nanoparticle, 210 mg of targeting moiety per gram of nucleic acid in the lipid nanoparticle, 211 mg of targeting moiety per gram of nucleic acid in the lipid nanoparticle, 212 mg of targeting moiety per gram of nucleic acid in the lipid nanoparticle, 213 mg of targeting moiety per gram of nucleic acid in the lipid nanoparticle, 214 mg of targeting moiety per gram of nucleic acid in the lipid nanoparticle, 215 mg of targeting moiety per gram of nucleic acid in the lipid nanoparticle, 216 mg of targeting moiety per gram of nucleic acid in the lipid nanoparticle, 217 mg of targeting moiety per gram of nucleic acid in the lipid nanoparticle, 218 mg of targeting moiety per gram of nucleic acid in the lipid nanoparticle, 219 mg of targeting moiety per gram of nucleic acid in the lipid nanoparticle, 220 mg of targeting moiety per gram of nucleic acid in the lipid nanoparticle, 221 mg of targeting moiety per gram of nucleic acid in the lipid nanoparticle, 222 mg of targeting moiety per gram of nucleic acid in the lipid nanoparticle, 223 mg of targeting moiety per gram of nucleic acid in the lipid nanoparticle, 224 mg of targeting moiety per gram of nucleic acid in the lipid nanoparticle, 225 mg of targeting moiety per gram of nucleic acid in the lipid nanoparticle, 226 mg of targeting moiety per gram of nucleic acid in the lipid nanoparticle, 227 mg of targeting moiety per gram of nucleic acid in the lipid nanoparticle, from 228 mg of targeting moiety per gram of nucleic acid in the lipid nanoparticle, 229 mg of targeting moiety per gram of nucleic acid in the lipid nanoparticle, 230 mg of targeting moiety per gram of nucleic acid in the lipid nanoparticle, 231 mg of targeting moiety per gram of nucleic acid in the lipid nanoparticle, 232 mg of targeting moiety per gram of nucleic acid in the lipid nanoparticle, 233 mg of targeting moiety per gram of nucleic acid in the lipid nanoparticle, 234 mg of targeting moiety per gram of nucleic acid in the lipid nanoparticle, 235 mg of targeting moiety per gram of nucleic acid in the lipid nanoparticle, 236 mg of targeting moiety per gram of nucleic acid in the lipid nanoparticle, 237 mg of targeting moiety per gram of nucleic acid in the lipid nanoparticle, 238 mg of targeting moiety per gram of nucleic acid in the lipid nanoparticle, 239 mg of targeting moiety per gram of nucleic acid in the lipid nanoparticle, 240 mg of targeting moiety per gram of nucleic acid in the lipid nanoparticle, 241 mg of targeting moiety per gram of nucleic acid in the lipid nanoparticle, 242 mg of targeting moiety per gram of nucleic acid in the lipid nanoparticle, 243 mg of targeting moiety per gram of nucleic acid in the lipid nanoparticle, 244 mg of targeting moiety per gram of nucleic acid in the lipid nanoparticle, 245 mg of targeting moiety per gram of nucleic acid in the lipid nanoparticle, 246 mg of targeting moiety per gram of nucleic acid in the lipid nanoparticle, 247 mg of targeting moiety per gram of nucleic acid in the lipid nanoparticle, 248 mg of targeting moiety per gram of nucleic acid in the lipid nanoparticle, 249 mg of targeting moiety per gram of nucleic acid in the lipid nanoparticle, 250 mg of targeting moiety per gram of nucleic acid in the lipid nanoparticle, 251 mg of targeting moiety per gram of nucleic acid in the lipid nanoparticle, 252 mg of targeting moiety per gram of nucleic acid in the lipid nanoparticle, 253 mg of targeting moiety per gram of nucleic acid in the lipid nanoparticle, 254 mg of targeting moiety per gram of nucleic acid in the lipid nanoparticle, 255 mg of targeting moiety per gram of nucleic acid in the lipid nanoparticle, 256 mg of targeting moiety per gram of nucleic acid in the lipid nanoparticle, 257 mg of targeting moiety per gram of nucleic acid in the lipid nanoparticle, 258 mg of targeting moiety per gram of nucleic acid in the lipid nanoparticle, 259 mg of targeting moiety per gram of nucleic acid in the lipid nanoparticle, 260 mg of targeting moiety per gram of nucleic acid in the lipid nanoparticle, 261 mg of targeting moiety per gram of nucleic acid in the lipid nanoparticle, 262 mg of targeting moiety per gram of nucleic acid in the lipid nanoparticle, 263 mg of targeting moiety per gram of nucleic acid in the lipid nanoparticle, 264 mg of targeting moiety per gram of nucleic acid in the lipid nanoparticle, 265 mg of targeting moiety per gram of nucleic acid in the lipid nanoparticle, 266 mg of targeting moiety per gram of nucleic acid in the lipid nanoparticle, 267 mg of targeting moiety per gram of nucleic acid in the lipid nanoparticle, 268 mg of targeting moiety per gram of nucleic acid in the lipid nanoparticle, 269 mg of targeting moiety per gram of nucleic acid in the lipid nanoparticle, 270 mg of targeting moiety per gram of nucleic acid in the lipid nanoparticle, 271 mg of targeting moiety per gram of nucleic acid in the lipid nanoparticle, 272 mg of targeting moiety per gram of nucleic acid in the lipid nanoparticle, 273 mg of targeting moiety per gram of nucleic acid in the lipid nanoparticle, 274 mg of targeting moiety per gram of nucleic acid in the lipid nanoparticle, 275 mg of targeting moiety per gram of nucleic acid in the lipid nanoparticle, 276 mg of targeting moiety per gram of nucleic acid in the lipid nanoparticle, 277 mg of targeting moiety per gram of nucleic acid in the lipid nanoparticle, 278 mg of targeting moiety per gram of nucleic acid in the lipid nanoparticle, 279 mg of targeting moiety per gram of nucleic acid in the lipid nanoparticle, 280 mg of targeting moiety per gram of nucleic acid in the lipid nanoparticle, 281 mg of targeting moiety per gram of nucleic acid in the lipid nanoparticle, 282 mg of targeting moiety per gram of nucleic acid in the lipid nanoparticle, 283 mg of targeting moiety per gram of nucleic acid in the lipid nanoparticle, 284 mg of targeting moiety per gram of nucleic acid in the lipid nanoparticle, 285 mg of targeting moiety per gram of nucleic acid in the lipid nanoparticle, 286 mg of targeting moiety per gram of nucleic acid in the lipid nanoparticle, 287 mg of targeting moiety per gram of nucleic acid in the lipid nanoparticle, 288 mg of targeting moiety per gram of nucleic acid in the lipid nanoparticle, 289 mg of targeting moiety per gram of nucleic acid in the lipid nanoparticle, 290 mg of targeting moiety per gram of nucleic acid in the lipid nanoparticle, 291 mg of targeting moiety per gram of nucleic acid in the lipid nanoparticle, 292 mg of targeting moiety per gram of nucleic acid in the lipid nanoparticle, 293 mg of targeting moiety per gram of nucleic acid in the lipid nanoparticle, 294 mg of targeting moiety per gram of nucleic acid in the lipid nanoparticle, 295 mg of targeting moiety per gram of nucleic acid in the lipid nanoparticle, 296 mg of targeting moiety per gram of nucleic acid in the lipid nanoparticle, 297 mg of targeting moiety per gram of nucleic acid in the lipid nanoparticle, 298 mg of targeting moiety per gram of nucleic acid in the lipid nanoparticle, 299 mg of targeting moiety per gram of nucleic acid in the lipid nanoparticle, 300 mg of targeting moiety per gram of nucleic acid in the lipid nanoparticle, 301 mg of targeting moiety per gram of nucleic acid in the lipid nanoparticle, 302 mg of targeting moiety per gram of nucleic acid in the lipid nanoparticle, 303 mg of targeting moiety per gram of nucleic acid in the lipid nanoparticle, 304 mg of targeting moiety per gram of nucleic acid in the lipid nanoparticle, 305 mg of targeting moiety per gram of nucleic acid in the lipid nanoparticle, 306 mg of targeting moiety per gram of nucleic acid in the lipid nanoparticle, 307 mg of targeting moiety per gram of nucleic acid in the lipid nanoparticle, 308 mg of targeting moiety per gram of nucleic acid in the lipid nanoparticle, 309 mg of targeting moiety per gram of nucleic acid in the lipid nanoparticle, 310 mg of targeting moiety per gram of nucleic acid in the lipid nanoparticle, 311 mg of targeting moiety per gram of nucleic acid in the lipid nanoparticle, 312 mg of targeting moiety per gram of nucleic acid in the lipid nanoparticle, 313 mg of targeting moiety per gram of nucleic acid in the lipid nanoparticle, 314 mg of targeting moiety per gram of nucleic acid in the lipid nanoparticle, 315 mg of targeting moiety per gram of nucleic acid in the lipid nanoparticle, 316 mg of targeting moiety per gram of nucleic acid in the lipid nanoparticle, 317 mg of targeting moiety per gram of nucleic acid in the lipid nanoparticle, 318 mg of targeting moiety per gram of nucleic acid in the lipid nanoparticle, 319 mg of targeting moiety per gram of nucleic acid in the lipid nanoparticle, 320 mg of targeting moiety per gram of nucleic acid in the lipid nanoparticle, 321 mg of targeting moiety per gram of nucleic acid in the lipid nanoparticle, 322 mg of targeting moiety per gram of nucleic acid in the lipid nanoparticle, 323 mg of targeting moiety per gram of nucleic acid in the lipid nanoparticle, 324 mg of targeting moiety per gram of nucleic acid in the lipid nanoparticle, 325 mg of targeting moiety per gram of nucleic acid in the lipid nanoparticle, 326 mg of targeting moiety per gram of nucleic acid in the lipid nanoparticle, 327 mg of targeting moiety per gram of nucleic acid in the lipid nanoparticle, 328 mg of targeting moiety per gram of nucleic acid in the lipid nanoparticle, 329 mg of targeting moiety per gram of nucleic acid in the lipid nanoparticle, 330 mg of targeting moiety per gram of nucleic acid in the lipid nanoparticle, 331 mg of targeting moiety per gram of nucleic acid in the lipid nanoparticle, 332 mg of targeting moiety per gram of nucleic acid in the lipid nanoparticle, 333 mg of targeting moiety per gram of nucleic acid in the lipid nanoparticle, 334 mg of targeting moiety per gram of nucleic acid in the lipid nanoparticle, 335 mg of targeting moiety per gram of nucleic acid in the lipid nanoparticle, 336 mg of targeting moiety per gram of nucleic acid in the lipid nanoparticle, 337 mg of targeting moiety per gram of nucleic acid in the lipid nanoparticle, 338 mg of targeting moiety per gram of nucleic acid in the lipid nanoparticle, 339 mg of targeting moiety per gram of nucleic acid in the lipid nanoparticle, 340 mg of targeting moiety per gram of nucleic acid in the lipid nanoparticle, 341 mg of targeting moiety per gram of nucleic acid in the lipid nanoparticle, 342 mg of targeting moiety per gram of nucleic acid in the lipid nanoparticle, 343 mg of targeting moiety per gram of nucleic acid in the lipid nanoparticle, 344 mg of targeting moiety per gram of nucleic acid in the lipid nanoparticle, 345 mg of targeting moiety per gram of nucleic acid in the lipid nanoparticle, 346 mg of targeting moiety per gram of nucleic acid in the lipid nanoparticle, 347 mg of targeting moiety per gram of nucleic acid in the lipid nanoparticle, 348 mg of targeting moiety per gram of nucleic acid in the lipid nanoparticle, 349 mg of targeting moiety per gram of nucleic acid in the lipid nanoparticle, or 350 mg of targeting moiety per gram of nucleic acid in the lipid nanoparticle. In some embodiments that can be combined with preceding embodiments, from about 1% to about 95%, from about 2% to about 95%, from about 3% to about 95%, from about 4% to about 95%, from about 5% to about 95%, from about 6% to about 95%, from about 7% to about 95%, from about 8% to about 95%, from about 9% to about 95%, from about 10% to about 95%, from about 11% to about 95%, from about 12% to about 95%, from about 13% to about 95%, from about 14% to about 95%, from about 15% to about 95%, from about 16% to about 95%, from about 17% to about 95%, from about 18% to about 95%, from about 19% to about 95%, from about 20% to about 95%, from about 21% to about 95%, from about 22% to about 95%, from about 23% to about 95%, from about 24% to about 95%, from about 25% to about 95%, from about 26% to about 95%, from about 27% to about 95%, from about 28% to about 95%, from about 29% to about 95%, from about 30% to about 95%, from about 31% to about 95%, from about 32% to about 95%, from about 33% to about 95%, from about 34% to about 95%, from about 35% to about 95%, from about 36% to about 95%, from about 37% to about 95%, from about 38% to about 95%, from about 39% to about 95%, from about 40% to about 95%, from about 41% to about 95%, from about 42% to about 95%, from about 43% to about 95%, from about 44% to about 95%, from about 45% to about 95%, from about 46% to about 95%, from about 47% to about 95%, from about 48% to about 95%, from about 49% to about 95%, from about 50% to about 95%, from about 51% to about 95%, from about 52% to about 95%, from about 53% to about 95%, from about 54% to about 95%, from about 55% to about 95%, from about 56% to about 95%, from about 57% to about 95%, from about 58% to about 95%, from about 59% to about 95%, from about 60% to about 95%, from about 61% to about 95%, from about 62% to about 95%, from about 63% to about 95%, from about 64% to about 95%, from about 65% to about 95%, from about 66% to about 95%, from about 67% to about 95%, from about 68% to about 95%, from about 69% to about 95%, from about 70% to about 95%, of the amount of the targeting moiety in the aforementioned ratios is incorporated into the lipid nanoparticle, thereby forming a targeted lipid nanoparticle.

In some embodiments that may be combined with preceding embodiments, the lipid nanoparticle comprises at least one ionizable lipid, cholesterol, at least one neutral lipid, and at least one polyethylene glycol (PEG)-lipid.

In some embodiments that may be combined with preceding embodiments, the ionizable lipid is selected from the group consisting of: a Lipid 1, a Lipid 2, a Lipid 3, a Lipid 4, a Lipid 5, a Lipid 6, a Lipid 7, a Lipid 8, a Lipid 9, a Lipid 10, a KC2 lipid, a DLin-MC2-DMA lipid, a DLin-MC3-DMA lipid, a DSDMA lipid, a DODMA lipid, a DLinDMA lipid, a DLenDMA lipid, a γ-DLenDMA lipid, a DLin-K-DMA lipid, a DLin-C2K-DMA lipid, a DLin-K-C3-DMA lipid, a DLin-K-C4-DMA lipid, a DLen-C2K-DMA lipid, a γ-DLen-C2K-DMA lipid, a DLin-MP-DMA lipid, and any combination thereof. In some embodiments, the ionizable lipid comprises a KC2 lipid. In some embodiments, the ionizable lipid comprises a Lipid 1 ionizable lipid. In some embodiments, the ionizable lipid comprises a Lipid 2 ionizable lipid. In some embodiments, the ionizable lipid comprises a Lipid 3 ionizable lipid. In some embodiments, the ionizable lipid comprises a Lipid 4 ionizable lipid. In some embodiments, the ionizable lipid comprises a Lipid 5 ionizable lipid. In some embodiments, the ionizable lipid comprises a Lipid 6 ionizable lipid. In some embodiments, the ionizable lipid comprises a Lipid 7 ionizable lipid. In some embodiments, the ionizable lipid comprises a Lipid 8 ionizable lipid. In some embodiments, the ionizable lipid comprises a Lipid 9 ionizable lipid. In some embodiments, the ionizable lipid comprises a Lipid 10 ionizable lipid. In some embodiments, the ionizable lipid comprises a DLin-MC2-DMA lipid. In some embodiments, the ionizable lipid comprises a DLin-MC3-DMA lipid. In some embodiments, the ionizable lipid comprises a DSDMA lipid. In some embodiments, the ionizable lipid comprises a DODMA lipid. In some embodiments, the ionizable lipid comprises a DLinDMA lipid. In some embodiments, the ionizable lipid comprises a DLenDMA lipid. In some embodiments, the ionizable lipid comprises a γ-DLenDMA lipid. In some embodiments, the ionizable lipid comprises a DLin-K-DMA lipid. In some embodiments, the ionizable lipid comprises a DLin-C2K-DMA lipid. In some embodiments, the ionizable lipid comprises a DLin-K-C3-DMA lipid. In some embodiments, the ionizable lipid comprises a DLin-K-C4-DMA lipid. In some embodiments, the ionizable lipid comprises a DLen-C2K-DMA lipid. In some embodiments, the ionizable lipid comprises a γ-DLen-C2K-DMA lipid. In some embodiments, the ionizable lipid comprises a DLin-MP-DMA lipid. Chemical structures of such ionizable lipids are depicted below:

### Lipid 1,

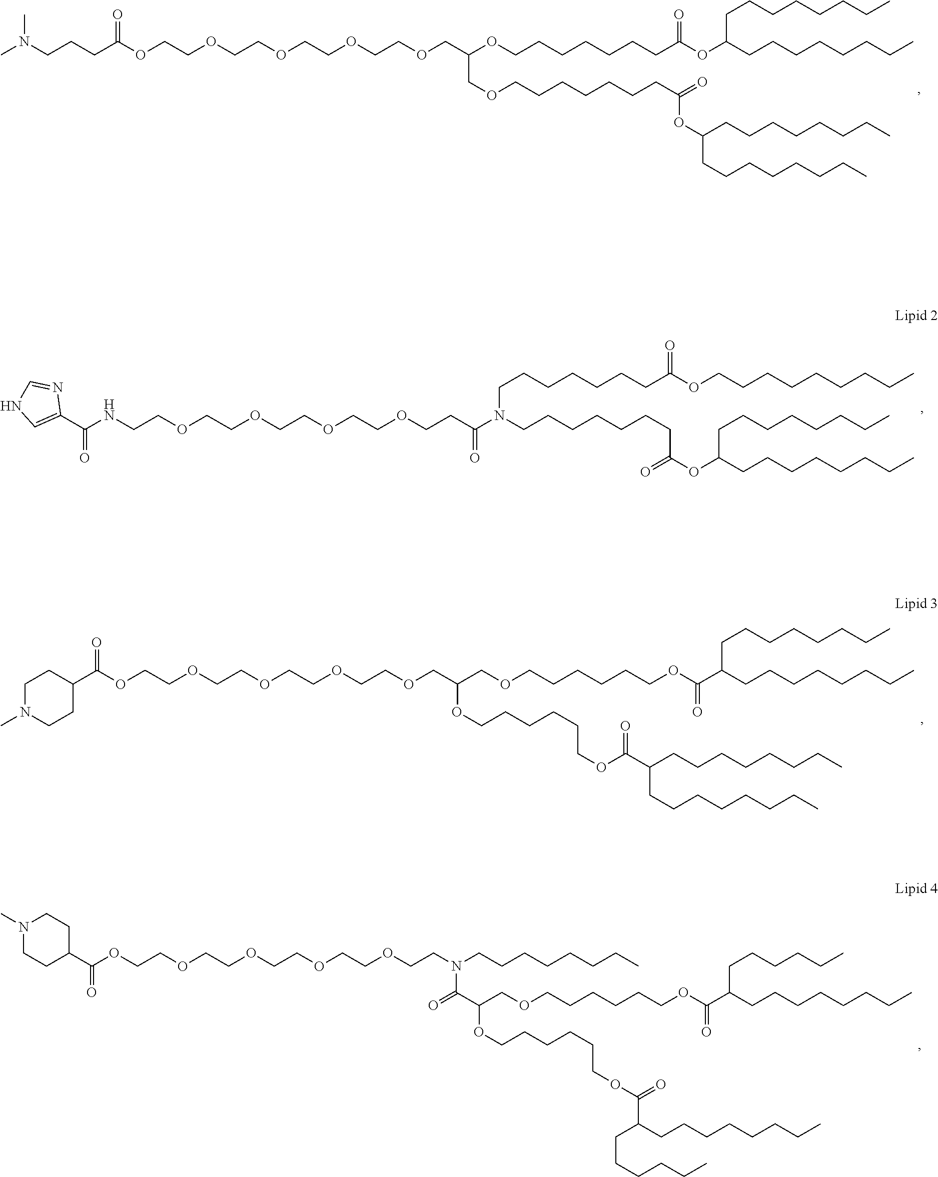

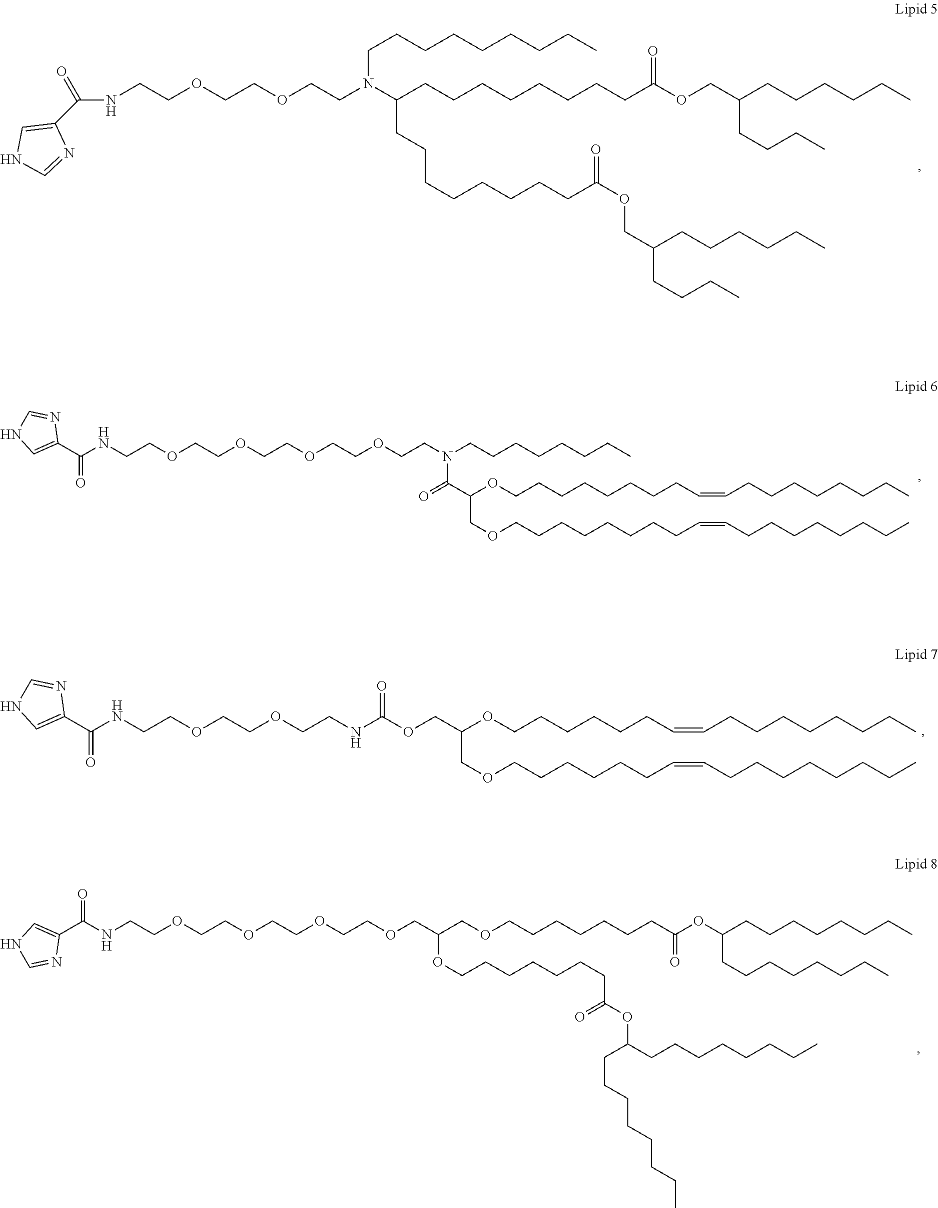 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC ; CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1 ; CCCCCCCC/C=C\CCCCCCOCC(COC(=O)NCCOCCOCCNC(=O)c1c[nH]cn1)OCCCCCC/C=C\CCCCCCCC ; CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

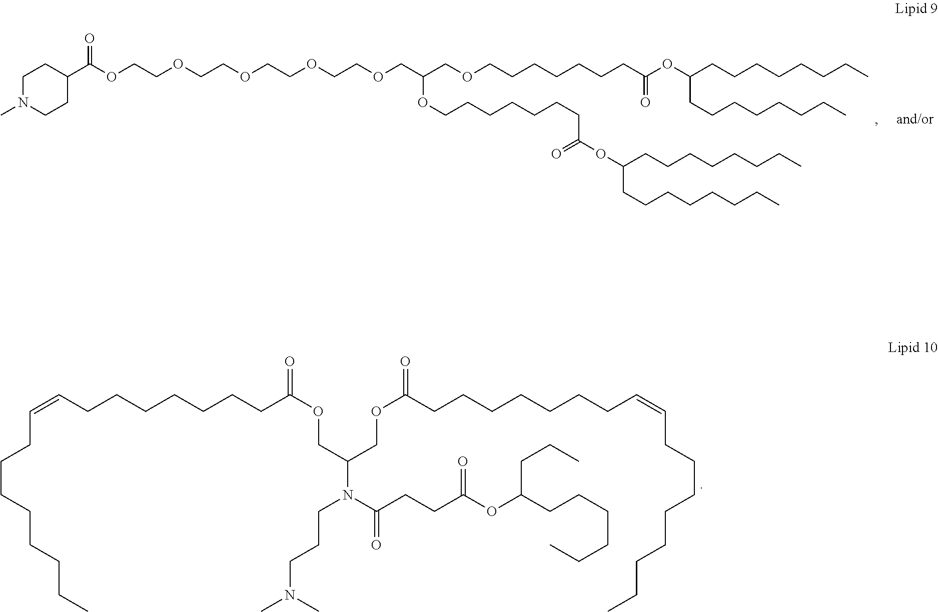 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC ; CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In some embodiments that may be combined with preceding embodiments, the neutral lipid comprises a phosphatidylcholine lipid, a phosphatidylethanolamine lipid, or a combination thereof. In some embodiments, the neutral lipid comprises a phosphatidylcholine lipid. In some embodiments, the neutral lipid comprises a phosphatidylethanolamine lipid. In some embodiments, the phosphatidylcholine lipid or the phosphatidylethanolamine lipid is selected from the group consisting of: 1,2-dioleoyl-sn-glycero-3-phosphoethanolamine (DOPE), 1,2-distearoyl-sn-glycero-3-phosphocoline (DSPC), 1,2-dipalmitoyl-sn-glycero-3-phosphocholine (DPPC), 1-palmitoyl-2-oleoyl-glycero-3-phosphocholine (POPC), and any combination thereof. In some embodiments, the neutral lipid comprises DOPE. In some embodiments, the neutral lipid comprises DSPC. In some embodiments, the neutral lipid comprises DPPC. In some embodiments, the neutral lipid comprises POPC.

In some embodiments that may be combined with preceding embodiments, the lipid nanoparticle comprises a first amount of the ionizable lipid, a second amount of the cholesterol, a third amount of the neutral lipid, and a fourth amount of the PEG-lipid.

In some embodiments that may be combined with preceding embodiments, the first amount is at least about 40%, 45%, 50%, 55%, 60%, or more of the molar mass of the lipid nanoparticle, at most about 60%, 55%, 50%, 45%, 40%, or less of the molar mass of the lipid nanoparticle, or a percentage of the molar mass of the lipid nanoparticle that is within a range defined by any two of the preceding values. For instance, in some embodiments, the first amount is between about 40% and about 45%, between about 40% and about 50%, between about 40% and about 55%, between about 40% and about 60%, between about 45% and about 50%, between about 45% and about 55%, between about 45% and about 60%, between about 50% and about 55%, between about 50% and about 60%, or between about 55% and about 60% of the molar mass of the lipid nanoparticle.

In some embodiments that may be combined with preceding embodiments, the third amount is at least about 10%, 15%, 20%, 25%, 30%, or more of the molar mass of the lipid nanoparticle, at most about 30%, 25%, 20%, 15%, 10%, or less of the molar mass of the lipid nanoparticle, or a percentage of the molar mass of the lipid nanoparticle that is within a range defined by any two of the preceding values. For instance, in some embodiments, the third amount is between about 10% and about 15%, between about 10% and about 20%, between about 10% and about 25%, between about 10% and about 30%, between about 15% and about 20%, between about 15% and about 25%, between about 15% and about 30%, between about 20% and about 25%, between about 20% and about 30%, or between about 25% and about 30% of the molar mass of the lipid nanoparticle.

In some embodiments that may be combined with preceding embodiments, the fourth amount is at least about 0.5%, 1%, 1.5%, 2%, 2.5%, 3%, 3.5%, 4%, 4.5%, 5%, or more of the molar mass of the lipid nanoparticle, at most about 5%, 4.5%, 4%, 3.5%, 3%, 2.5%, 2%, 1.5%, 1%, 0.5%, or less of the molar mass of the lipid nanoparticle, or a percentage of the molar mass of the lipid nanoparticle that is within a range defined by any two of the preceding values. For instance, in some embodiments, the third amount is between about 0.5% and about 1%, between about 0.5% and about 1.5%, between about 0.5% and about 2%, between about 0.5% and about 2.5%, between about 0.5% and about 3%, between about 0.5% and about 3.5%, between about 0.5% and about 4%, between about 0.5% and about 4.5%, between about 0.5% and about 5%, between about 1% and about 1.5%, between about 1% and about 2%, between about 1% and about 2.5%, between about 1% and about 3%, between about 1% and about 3.5%, between about 1% and about 4%, between about 1% and about 4.5%, between about 1% and about 5%, between about 1.5% and about 2%, between about 1.5% and about 2.5%, between about 1.5% and about 3%, between about 1.5% and about 3.5%, between about 1.5% and about 4%, between about 1.5% and about 4.5%, between about 1.5% and about 5%, between about 2% and about 2.5%, between about 2% and about 3%, between about 2% and about 3.5%, between about 2% and about 4%, between about 2% and about 4.5%, between about 2% and about 5%, between about 2.5% and about 3%, between about 2.5% and about 3.5%, between about 2.5% and about 4%, between about 2.5% and about 4.5%, between about 2.5% and about 5%, between about 3% and about 3.5%, between about 3% and about 4%, between about 3% and about 4.5%, between about 3% and about 5%, between about 3.5% and about 4%, between about 3.5% and about 4.5%, between about 3.5% and about 5%, between about 4% and about 4.5%, between about 4% and about 5%, or between about 4.5% and about 5% of the molar mass of the lipid nanoparticle.

In some embodiments that may be combined with preceding embodiments, the first, second, third, and fourth amounts sum to 100% of the molar mass of the lipid nanoparticle. In some embodiments, the second amount is chosen to cause the first, second, third, and fourth amounts to sum to 100% when the first, third, and fourth amounts fail to sum to 100% on their own. For instance, when the first amount is 40%, the third amount is 30%, and the fourth amount is 5%, the second amount is 25% (for a sum total of 100%).

In some embodiments that may be combined with preceding embodiments, the lipid nanoparticle comprises a nucleic acid cargo. In some embodiments, the nucleic acid cargo in encapsulated into the lipid nanoparticle prior to the reaction or the insertion of the targeting moiety into the lipid nanoparticle. In some embodiments, the nucleic acid cargo in encapsulated into the lipid nanoparticle simultaneously with the reaction or the insertion of the targeting moiety into the lipid nanoparticle. In some embodiments, the nucleic acid cargo in encapsulated into the lipid nanoparticle after the reaction or the insertion of the targeting moiety into the lipid nanoparticle. In some embodiments, the reaction or the insertion of the targeting moiety into the lipid nanoparticle does not fragment the nucleic acid cargo. In some embodiments, the encapsulation of the nucleic acid cargo into the lipid nanoparticle has an encapsulation efficiency of at least about 50%, 55%, 60%, 65%, 70%, 75%, 80%, 85%, 90%, 95%, or more, at most about 95%, 90%, 85%, 80%, 75%, 70%, 65%, 60%, 55%, 50%, or less, or an encapsulation efficiency that is within a range defined by any two of the preceding values.

In some embodiments that may be combined with preceding embodiments, the lipid nanoparticle is characterized by a size, such as a diameter, length, width, or height. In some embodiments, the size is at least about 50 nanometers (nm), 55 nm, 60 nm, 65 nm, 70 nm, 75 nm, 80 nm, 85 nm, 90 nm, 95 nm, 100 nm, 105 nm, 110 nm, 115 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, or more, at most about 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm, 50 nm, or less, or within a range defined by any two of the preceding values. For instances, in some embodiments, the size is between about 50 nm and about 55 nm, between about 50 nm and about 60 nm, between about 50 nm and about 65 nm, between about 50 nm and about 70 nm, between about 50 nm and about 75 nm, between about 50 nm and about 80 nm, between about 50 nm and about 85 nm, between about 50 nm and about 90 nm, between about 50 nm and about 95 nm, between about 50 nm and about 100 nm, between about 50 nm and about 105 nm, between about 50 nm and about 110 nm, between about 50 nm and about 115 nm, between about 50 nm and about 120 nm, between about 50 nm and about 125 nm, between 50 nm and about 130 nm, between about 50 nm and about 135 nm, between about 50 nm and about 140 nm, between about 50 nm and about 145 nm, between about 50 nm and about 150 nm, between about 50 nm and about 155 nm, between about 50 nm and about 160 nm, between about 50 nm and about 165 nm, between about 50 nm and about 170 nm, between about 50 nm and about 175 nm, between about 50 nm and about 180 nm, between about 50 nm and about 185 nm, between about 50 nm and about 190 nm, between about 50 nm and about 195 nm, between about 50 nm and about 200 nm, between about 55 nm and about 60 nm, between about 55 nm and about 65 nm, between about 55 nm and about 70 nm, between about 55 nm and about 75 nm, between about 55 nm and about 80 nm, between about 55 nm and about 85 nm, between about 55 nm and about 90 nm, between about 55 nm and about 95 nm, between about 55 nm and about 100 nm, between about 55 nm and about 105 nm, between about 55 nm and about 110 nm, between about 55 nm and about 115 nm, between about 55 nm and about 120 nm, between about 55 nm and about 125 nm, between 55 nm and about 130 nm, between about 55 nm and about 135 nm, between about 55 nm and about 140 nm, between about 55 nm and about 145 nm, between about 55 nm and about 150 nm, between about 55 nm and about 155 nm, between about 55 nm and about 160 nm, between about 55 nm and about 165 nm, between about 55 nm and about 170 nm, between about 55 nm and about 175 nm, between about 55 nm and about 180 nm, between about 55 nm and about 185 nm, between about 55 nm and about 190 nm, between about 55 nm and about 195 nm, between about 55 nm and about 200 nm, between about 60 nm and about 65 nm, between about 60 nm and about 70 nm, between about 60 nm and about 75 nm, between about 60 nm and about 80 nm, between about 60 nm and about 85 nm, between about 60 nm and about 90 nm, between about 60 nm and about 95 nm, between about 60 nm and about 100 nm, between about 60 nm and about 105 nm, between about 60 nm and about 110 nm, between about 60 nm and about 115 nm, between about 60 nm and about 120 nm, between about 60 nm and about 125 nm, between 60 nm and about 130 nm, between about 60 nm and about 135 nm, between about 60 nm and about 140 nm, between about 60 nm and about 145 nm, between about 60 nm and about 150 nm, between about 60 nm and about 155 nm, between about 60 nm and about 160 nm, between about 60 nm and about 165 nm, between about 60 nm and about 170 nm, between about 60 nm and about 175 nm, between about 60 nm and about 180 nm, between about 60 nm and about 185 nm, between about 60 nm and about 190 nm, between about 60 nm and about 195 nm, between about 60 nm and about 200 nm, between about 65 nm and about 70 nm, between about 65 nm and about 75 nm, between about 65 nm and about 80 nm, between about 65 nm and about 85 nm, between about 65 nm and about 90 nm, between about 65 nm and about 95 nm, between about 65 nm and about 100 nm, between about 65 nm and about 105 nm, between about 65 nm and about 110 nm, between about 65 nm and about 115 nm, between about 65 nm and about 120 nm, between about 65 nm and about 125 nm, between 65 nm and about 130 nm, between about 65 nm and about 135 nm, between about 65 nm and about 140 nm, between about 65 nm and about 145 nm, between about 65 nm and about 150 nm, between about 65 nm and about 155 nm, between about 65 nm and about 160 nm, between about 65 nm and about 165 nm, between about 65 nm and about 170 nm, between about 65 nm and about 175 nm, between about 65 nm and about 180 nm, between about 65 nm and about 185 nm, between about 65 nm and about 190 nm, between about 65 nm and about 195 nm, between about 65 nm and about 200 nm, between about 70 nm and about 75 nm, between about 70 nm and about 80 nm, between about 70 nm and about 85 nm, between about 70 nm and about 90 nm, between about 70 nm and about 95 nm, between about 70 nm and about 100 nm, between about 70 nm and about 105 nm, between about 70 nm and about 110 nm, between about 70 nm and about 115 nm, between about 70 nm and about 120 nm, between about 70 nm and about 125 nm, between 70 nm and about 130 nm, between about 70 nm and about 135 nm, between about 70 nm and about 140 nm, between about 70 nm and about 145 nm, between about 70 nm and about 150 nm, between about 70 nm and about 155 nm, between about 70 nm and about 160 nm, between about 70 nm and about 165 nm, between about 70 nm and about 170 nm, between about 70 nm and about 175 nm, between about 70 nm and about 180 nm, between about 70 nm and about 185 nm, between about 70 nm and about 190 nm, between about 70 nm and about 195 nm, between about 70 nm and about 200 nm, between about 75 nm and about 80 nm, between about 75 nm and about 85 nm, between about 75 nm and about 90 nm, between about 75 nm and about 95 nm, between about 75 nm and about 100 nm, between about 75 nm and about 105 nm, between about 75 nm and about 110 nm, between about 75 nm and about 115 nm, between about 75 nm and about 120 nm, between about 75 nm and about 125 nm, between 75 nm and about 130 nm, between about 75 nm and about 135 nm, between about 75 nm and about 140 nm, between about 75 nm and about 145 nm, between about 75 nm and about 150 nm, between about 75 nm and about 155 nm, between about 75 nm and about 160 nm, between about 75 nm and about 165 nm, between about 75 nm and about 170 nm, between about 75 nm and about 175 nm, between about 75 nm and about 180 nm, between about 75 nm and about 185 nm, between about 75 nm and about 190 nm, between about 75 nm and about 195 nm, between about 75 nm and about 200 nm, between about 80 nm and about 85 nm, between about 80 nm and about 90 nm, between about 80 nm and about 95 nm, between about 80 nm and about 100 nm, between about 80 nm and about 105 nm, between about 80 nm and about 110 nm, between about 80 nm and about 115 nm, between about 80 nm and about 120 nm, between about 80 nm and about 125 nm, between 80 nm and about 130 nm, between about 80 nm and about 135 nm, between about 80 nm and about 140 nm, between about 80 nm and about 145 nm, between about 80 nm and about 150 nm, between about 80 nm and about 155 nm, between about 80 nm and about 160 nm, between about 80 nm and about 165 nm, between about 80 nm and about 170 nm, between about 80 nm and about 175 nm, between about 80 nm and about 180 nm, between about 80 nm and about 185 nm, between about 80 nm and about 190 nm, between about 80 nm and about 195 nm, between about 80 nm and about 200 nm, between about 85 nm and about 90 nm, between about 85 nm and about 95 nm, between about 85 nm and about 100 nm, between about 85 nm and about 105 nm, between about 85 nm and about 110 nm, between about 85 nm and about 115 nm, between about 85 nm and about 120 nm, between about 85 nm and about 125 nm, between 85 nm and about 130 nm, between about 85 nm and about 135 nm, between about 85 nm and about 140 nm, between about 85 nm and about 145 nm, between about 85 nm and about 150 nm, between about 85 nm and about 155 nm, between about 85 nm and about 160 nm, between about 85 nm and about 165 nm, between about 85 nm and about 170 nm, between about 85 nm and about 175 nm, between about 85 nm and about 180 nm, between about 85 nm and about 185 nm, between about 85 nm and about 190 nm, between about 85 nm and about 195 nm, between about 85 nm and about 200 nm, between about 90 nm and about 95 nm, between about 90 nm and about 100 nm, between about 90 nm and about 105 nm, between about 90 nm and about 110 nm, between about 90 nm and about 115 nm, between about 90 nm and about 120 nm, between about 90 nm and about 125 nm, between 90 nm and about 130 nm, between about 90 nm and about 135 nm, between about 90 nm and about 140 nm, between about 90 nm and about 145 nm, between about 90 nm and about 150 nm, between about 90 nm and about 155 nm, between about 90 nm and about 160 nm, between about 90 nm and about 165 nm, between about 90 nm and about 170 nm, between about 90 nm and about 175 nm, between about 90 nm and about 180 nm, between about 90 nm and about 185 nm, between about 90 nm and about 190 nm, between about 90 nm and about 195 nm, between about 90 nm and about 200 nm, between about 95 nm and about 100 nm, between about 95 nm and about 105 nm, between about 95 nm and about 110 nm, between about 95 nm and about 115 nm, between about 95 nm and about 120 nm, between about 95 nm and about 125 nm, between 95 nm and about 130 nm, between about 95 nm and about 135 nm, between about 95 nm and about 140 nm, between about 95 nm and about 145 nm, between about 95 nm and about 150 nm, between about 95 nm and about 155 nm, between about 95 nm and about 160 nm, between about 95 nm and about 165 nm, between about 95 nm and about 170 nm, between about 95 nm and about 175 nm, between about 95 nm and about 180 nm, between about 95 nm and about 185 nm, between about 95 nm and about 190 nm, between about 95 nm and about 195 nm, between about 95 nm and about 200 nm, between about 100 nm and about 105 nm, between about 100 nm and about 110 nm, between about 100 nm and about 115 nm, between about 100 nm and about 120 nm, between about 100 nm and about 125 nm, between 100 nm and about 130 nm, between about 100 nm and about 135 nm, between about 100 nm and about 140 nm, between about 100 nm and about 145 nm, between about 100 nm and about 150 nm, between about 100 nm and about 155 nm, between about 100 nm and about 160 nm, between about 100 nm and about 165 nm, between about 100 nm and about 170 nm, between about 100 nm and about 175 nm, between about 100 nm and about 180 nm, between about 100 nm and about 185 nm, between about 100 nm and about 190 nm, between about 100 nm and about 195 nm, between about 100 nm and about 200 nm, between about 105 nm and about 110 nm, between about 105 nm and about 115 nm, between about 105 nm and about 120 nm, between about 105 nm and about 125 nm, between 105 nm and about 130 nm, between about 105 nm and about 135 nm, between about 105 nm and about 140 nm, between about 105 nm and about 145 nm, between about 105 nm and about 150 nm, between about 105 nm and about 155 nm, between about 105 nm and about 160 nm, between about 105 nm and about 165 nm, between about 105 nm and about 170 nm, between about 105 nm and about 175 nm, between about 105 nm and about 180 nm, between about 105 nm and about 185 nm, between about 105 nm and about 190 nm, between about 105 nm and about 195 nm, between about 105 nm and about 200 nm, between about 110 nm and about 115 nm, between about 110 nm and about 120 nm, between about 110 nm and about 125 nm, between 110 nm and about 130 nm, between about 110 nm and about 135 nm, between about 110 nm and about 140 nm, between about 110 nm and about 145 nm, between about 110 nm and about 150 nm, between about 110 nm and about 155 nm, between about 110 nm and about 160 nm, between about 110 nm and about 165 nm, between about 110 nm and about 170 nm, between about 110 nm and about 175 nm, between about 110 nm and about 180 nm, between about 110 nm and about 185 nm, between about 110 nm and about 190 nm, between about 110 nm and about 195 nm, between about 110 nm and about 200 nm, between about 115 nm and about 120 nm, between about 115 nm and about 125 nm, between 115 nm and about 130 nm, between about 115 nm and about 135 nm, between about 115 nm and about 140 nm, between about 115 nm and about 145 nm, between about 115 nm and about 150 nm, between about 115 nm and about 155 nm, between about 115 nm and about 160 nm, between about 115 nm and about 165 nm, between about 115 nm and about 170 nm, between about 115 nm and about 175 nm, between about 115 nm and about 180 nm, between about 115 nm and about 185 nm, between about 115 nm and about 190 nm, between about 115 nm and about 195 nm, between about 115 nm and about 200 nm, between about 120 nm and about 125 nm, between 120 nm and about 130 nm, between about 120 nm and about 135 nm, between about 120 nm and about 140 nm, between about 120 nm and about 145 nm, between about 120 nm and about 150 nm, between about 120 nm and about 155 nm, between about 120 nm and about 160 nm, between about 120 nm and about 165 nm, between about 120 nm and about 170 nm, between about 120 nm and about 175 nm, between about 120 nm and about 180 nm, between about 120 nm and about 185 nm, between about 120 nm and about 190 nm, between about 120 nm and about 195 nm, between about 120 nm and about 200 nm, between 125 nm and about 130 nm, between about 125 nm and about 135 nm, between about 125 nm and about 140 nm, between about 125 nm and about 145 nm, between about 125 nm and about 150 nm, between about 125 nm and about 155 nm, between about 125 nm and about 160 nm, between about 125 nm and about 165 nm, between about 125 nm and about 170 nm, between about 125 nm and about 175 nm, between about 125 nm and about 180 nm, between about 125 nm and about 185 nm, between about 125 nm and about 190 nm, between about 125 nm and about 195 nm, between about 125 nm and about 200 nm, between about 130 nm and about 135 nm, between about 130 nm and about 140 nm, between about 130 nm and about 145 nm, between about 130 nm and about 150 nm, between about 130 nm and about 155 nm, between about 130 nm and about 160 nm, between about 130 nm and about 165 nm, between about 130 nm and about 170 nm, between about 130 nm and about 175 nm, between about 130 nm and about 180 nm, between about 130 nm and about 185 nm, between about 130 nm and about 190 nm, between about 130 nm and about 195 nm, between about 130 nm and about 200 nm, between about 135 nm and about 140 nm, between about 135 nm and about 145 nm, between about 135 nm and about 150 nm, between about 135 nm and about 155 nm, between about 135 nm and about 160 nm, between about 135 nm and about 165 nm, between about 135 nm and about 170 nm, between about 135 nm and about 175 nm, between about 135 nm and about 180 nm, between about 135 nm and about 185 nm, between about 135 nm and about 190 nm, between about 135 nm and about 195 nm, between about 135 nm and about 200 nm, between about 140 nm and about 145 nm, between about 140 nm and about 150 nm, between about 140 nm and about 155 nm, between about 140 nm and about 160 nm, between about 140 nm and about 165 nm, between about 140 nm and about 170 nm, between about 140 nm and about 175 nm, between about 140 nm and about 180 nm, between about 140 nm and about 185 nm, between about 140 nm and about 190 nm, between about 140 nm and about 195 nm, between about 140 nm and about 200 nm, between about 145 nm and about 150 nm, between about 145 nm and about 155 nm, between about 145 nm and about 160 nm, between about 145 nm and about 165 nm, between about 145 nm and about 170 nm, between about 145 nm and about 175 nm, between about 145 nm and about 180 nm, between about 145 nm and about 185 nm, between about 145 nm and about 190 nm, between about 145 nm and about 195 nm, between about 145 nm and about 200 nm, between about 150 nm and about 155 nm, between about 150 nm and about 160 nm, between about 150 nm and about 165 nm, between about 150 nm and about 170 nm, between about 150 nm and about 175 nm, between about 150 nm and about 180 nm, between about 150 nm and about 185 nm, between about 150 nm and about 190 nm, between about 150 nm and about 195 nm, between about 150 nm and about 200 nm, between about 155 nm and about 160 nm, between about 155 nm and about 165 nm, between about 155 nm and about 170 nm, between about 155 nm and about 175 nm, between about 155 nm and about 180 nm, between about 155 nm and about 185 nm, between about 155 nm and about 190 nm, between about 155 nm and about 195 nm, between about 155 nm and about 200 nm, between about 160 nm and about 165 nm, between about 160 nm and about 170 nm, between about 160 nm and about 175 nm, between about 160 nm and about 180 nm, between about 160 nm and about 185 nm, between about 160 nm and about 190 nm, between about 160 nm and about 195 nm, between about 160 nm and about 200 nm, between about 165 nm and about 170 nm, between about 165 nm and about 175 nm, between about 165 nm and about 180 nm, between about 165 nm and about 185 nm, between about 165 nm and about 190 nm, between about 165 nm and about 195 nm, between about 165 nm and about 200 nm, between about 170 nm and about 175 nm, between about 170 nm and about 180 nm, between about 170 nm and about 185 nm, between about 170 nm and about 190 nm, between about 170 nm and about 195 nm, between about 170 nm and about 200 nm, between about 175 nm and about 180 nm, between about 175 nm and about 185 nm, between about 175 nm and about 190 nm, between about 175 nm and about 195 nm, between about 175 nm and about 200 nm, between about 180 nm and about 185 nm, between about 180 nm and about 190 nm, between about 180 nm and about 195 nm, between about 180 nm and about 200 nm, between about 185 nm and about 190 nm, between about 185 nm and about 195 nm, between about 185 nm and about 200 nm, between about 190 nm and about 195 nm, between about 190 nm and about 200 nm, or between about 195 nm and about 200.

In some embodiments that may be combined with preceding embodiments, the reaction or the insertion of the targeting moiety into the lipid nanoparticle increases the size of the lipid nanoparticle. In some embodiments, the reaction or the insertion of the targeting moiety into the lipid nanoparticle increases the size of the lipid nanoparticle by at least about 5%, 10%, 15%, 20%, 25%, 30%, 35%, 40%, 45%, 50%, or more, at most about 50%, 45%, 40%, 35%, 30%, 25%, 20%, 15%, 10%, 5%, or less, or an amount that is within a range defined by any two of the preceding values. For instance, in some embodiments, the reaction or the insertion of the targeting moiety into the lipid nanoparticle increases the size of the lipid nanoparticle by between about 5% and about 10%, between about 5% and about 15%, between about 5% and about 20%, between about 5% and about 25%, between about 5% and about 30%, between about 5% and about 35%, between about 5% and about 40%, between about 5% and about 45%, between about 5% and about 50%, between about 10% and about 15%, between about 10% and about 20%, between about 10% and about 25%, between about 10% and about 30%, between about 10% and about 35%, between about 10% and about 40%, between about 10% and about 45%, between about 10% and about 50%, between about 15% and about 20%, between about 15% and about 25%, between about 15% and about 30%, between about 15% and about 35%, between about 15% and about 40%, between about 15% and about 45%, between about 15% and about 50%, between about 20% and about 25%, between about 20% and about 30%, between about 20% and about 35%, between about 20% and about 40%, between about 20% and about 45%, between about 20% and about 50%, between about 25% and about 30%, between about 25% and about 35%, between about 25% and about 40%, between about 25% and about 45%, between about 25% and about 50%, between about 30% and about 35%, between about 30% and about 40%, between about 30% and about 45%, between about 30% and about 50%, between about 35% and about 40%, between about 35% and about 45%, between about 35% and about 50%, between about 40% and about 45%, between about 40% and about 50%, or between about 45% and about 50%.

In some embodiments that may be combined with preceding embodiments, the reaction or the insertion of the targeting moiety is performed under rotation. That is, in some embodiments, the reaction or the insertion of the targeting moiety occurs at a rotational speed. In some embodiments, the rotational speed is at least about 0 revolutions per minute (rpm), 100 rpm, 200 rpm, 300 rpm, 400 rpm, 500 rpm, 600 rpm, 700 rpm, 800 rpm, 900 rpm, 1,000 rpm, or more, at most about 1,000 rpm, 900 rpm, 800 rpm, 700 rpm, 600 rpm, 500 rpm, 400 rpm, 300 rpm, 200 rpm, 100 rpm, or 0 rpm, or within a range defined by any two of the preceding values.

In many embodiments described herein the disclosure provides a process for the insertion of a targeting moiety into a lipid nanoparticle, the process comprising the steps of inserting the targeting moiety into the lipid nanoparticle in a reaction that combines at least 0.15 μg of the targeting moiety per gram of the total lipids in the lipid nanoparticle for a time-period of at least one hour, optionally under a temperature that does not exceed 54° C., thereby forming an amount of targeted nanoparticles; and rapidly quenching the insertion of the targeting moiety by cooling the reaction to about 20° C. or lower. In the example shown, a quenching operation is conducted by lowering the temperature of the reaction in which the insertion of the targeting moiety into the lipid nanoparticle is occurring at 120. The lowering of the temperature quenches the insertion of the moiety. In some embodiments, the quenching operation comprises cooling the lipid nanoparticle (and the targeting moiety contained therein) to a quenching temperature rapidly, e.g., within milliseconds, seconds, or minutes. In some embodiments, the quenching operation is conducted using a heat exchanger. In some embodiments, the quenching temperature is at least about 0° C., 5° C., 10° C., 15° C., 20° C., or more, at most about 20° C., 15° C., 10° C., 5° C., 0° C., or less, or within a range defined by any two of the preceding values compared to the temperature for insertion of the targeting moiety. For instance, in some embodiments, the quenching temperature is between about 0° C. and about 5° C., between about 0° C. and about 10° C., between about 0° C. and about 15° C., between about 0° C. and about 20° C., between about 5° C. and about 10° C., between about 5° C. and about 15° C., between about 5° C. and about 20° C., between about 10° C. and about 15° C., between about 10° C. and about 20° C., or between about 15° C. and about 20° C. lower than the temperature used for the insertion of the targeting moiety.

In some embodiments that can be combined with the preceding embodiments, the quenching operation is carried out over a period of time. In some embodiments, the period of time is no more than 5 minutes, no more than 4 minutes, no more than 3 minutes, no more than 2 minutes, no more than 1 minute, or no more than 30 seconds. In some embodiments, the period of time is at least about 5 minutes (min), 10 min, 15 min, 20 min, or more, at most about 20 min, 10 min, 5 min, or less, or within a range defined by any two of the preceding values. For instance, in some embodiments, the period of time is between about 5 min and about 10 min, between about 5 min and about 15 min, between about 5 min and about 20 min, between about 10 min and about 15 min, between about 10 min and about 20 min, or between about 15 min and about 20 min.

In some embodiments, the process 100, or any one or both of operations 110 and 120, yields an amount of targeted nanoparticles. In some embodiments, the amount of nanoparticles is a percentage of the amount of lipid nanoparticles used in operation 110. In some embodiments, the percentage is at least about 60%, 65%, 70%, 75%, 80%, 85%, 90%, 95%, or more, at most about 95%, 90%, 85%, 80%, 75%, 70%, 65%, 60%, or less, or a percentage that is within a range defined by any two of the preceding values.

In some embodiments, the targeted nanoparticles are stable (i.e., at least about 50%, 55%, 60%, 65%, 70%, 75%, 80%, 85%, 90%, 95%, or more of the targeting moieties remain contained within the lipid nanoparticles) for a period of time while stored at a storage temperature. In some embodiments, the period of time is at least about 1 h, 2 h, 3 h, 4 h, 6 h, 8 h, 12 h, 1 day (d), 2 d, 3 d, 4 d, 5 d, 6 d, 1 week (w), 2 w, 3 w, 4 w, or more, at most about 4 w, 3 w, 2 w, 1 w, 6 d, 5 d, 4 d, 3 d, 2 d, 1 d, 12 h, 8 h, 6 h, 4 h, 3 h, 2 h, 1 h, or less, or within a range defined by any two of the preceding values. For instance, in some embodiments, the period of time is between about 1 d and about 2 d, about 1 d and about 4 d, about 1 d and about 1 w, about 1 d and about 2 w, about 1 d and about 4 w, about 2 d and about 4 d, about 2 d and about 1 w, about 2 d and about 2 w, about 2 d and about 4 w, about 4 d and about 1 w, about 4 d and about 2 w, about 4 d and about 4 w, about 1 w and about 2 w, about 1 w and about 4 w, or about 2 w and about 4 w. In some embodiments, the storage temperature is at least about 0° C., 2° C., 4° C., 6° C., 8° C., 10° C., 12° C., 14° C., 16° C., 18° C., 20° C., or more, at most about 20° C., 18° C., 16° C., 14° C., 12° C., 10° C., 8° C., 6° C., 4° C., 2° C., 0° C., or less, or within a range defined by any two of the preceding values. For instance, in some embodiments, the storage temperature is between about 0° C. and about 2° C., about 0° C. and about 4° C., about 0° C. and about 6° C., about 0° C. and about 8° C., about 0° C. and about 10° C., about 0° C. and about 12° C., about 0° C. and about 14° C., about 0° C. and about 16° C., about 0° C. and about 18° C., about 0° C. and about 20° C., about 2° C. and about 4° C., about 2° C. and about 6° C., about 2° C. and about 8° C., about 2° C. and about 10° C., about 2° C. and about 12° C., about 2° C. and about 14° C., about 2° C. and about 16° C., about 2° C. and about 18° C., about 2° C. and about 20° C., about 4° C. and about 6° C., about 4° C. and about 8° C., about 4° C. and about 10° C., about 4° C. and about 12° C., about 4° C. and about 14° C., about 4° C. and about 16° C., about 4° C. and about 18° C., about 4° C. and about 20° C., about 6° C. and about 8° C., about 6° C. and about 10° C., about 6° C. and about 12° C., about 6° C. and about 14° C., about 6° C. and about 16° C., about 6° C. and about 18° C., about 6° C. and about 20° C., about 8° C. and about 10° C., about 8° C. and about 12° C., about 8° C. and about 14° C., about 8° C. and about 16° C., about 8° C. and about 18° C., about 8° C. and about 20° C., about 10° C. and about 12° C., about 10° C. and about 14° C., about 10° C. and about 16° C., about 10° C. and about 18° C., about 10° C. and about 20° C., about 12° C. and about 14° C., about 12° C. and about 16° C., about 12° C. and about 18° C., about 12° C. and about 20° C., about 14° C. and about 16° C., about 14° C. and about 18° C., about 14° C. and about 20° C., about 16° C. and about 18° C., about 16° C. and about 20° C., or about 18° C. and about 20° C. In some embodiments, the process 100 further comprises storing the targeted nanoparticles for the period of time at the storage temperature.

In some aspects, the disclosure provides a process for the insertion of a targeting moiety into a lipid nanoparticle, the process comprising the steps of: inserting the targeting moiety into the lipid nanoparticle in a reaction that combines 0.15 micrograms amount of a targeting moiety per mole of the total lipids in a lipid nanoparticle for a time-period of at least one hour at a temperature no higher than 42° C., thereby forming an amount of targeted lipid nanoparticles; and quenching the insertion of the targeting moiety by cooling the reaction to about 20° C. or lower.

### IV. Targeted Lipid Nanoparticle

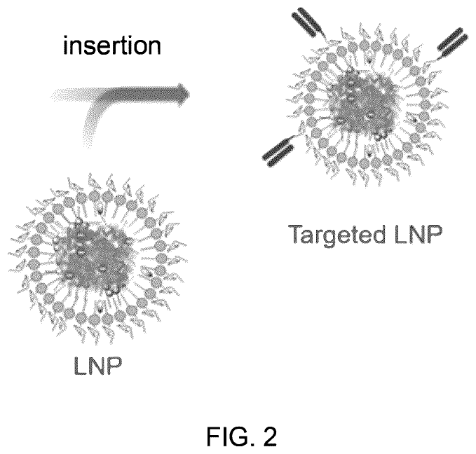

FIG. 2 is a schematic of a composition comprising a targeted lipid nanoparticle 200. In the example shown, the targeted lipid nanoparticle 200 comprises a lipid nanoparticle 210 and a targeting moiety 220 contained therein. In some embodiments, the targeted lipid nanoparticle 200 is produced by the process 100, or by any one or more of operations 110 and 120, described herein with respect to process 100 of FIG. 1. In some embodiments, the lipid nanoparticle 210 comprises any lipid nanoparticle described herein with respect to process 100 of FIG. 1. In some embodiments, the targeting moiety 220 comprises any targeting moiety described herein with respect to process 100 of FIG. 1.

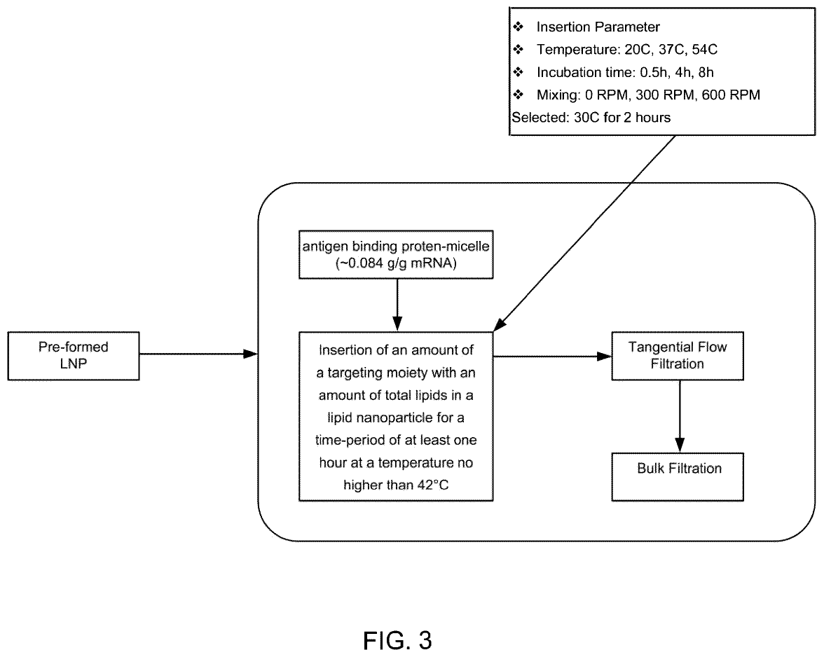

The following describes the process development for evaluation the impart of temperature, incubation time, and mixing parameters into the incorporation of a targeting moiety into a lipid nanoparticle. FIG. 3 is a schematic of various parameters for inserting a targeting moiety into a lipid nanoparticle in a reaction. The schematics illustrate a pre-formed LNP with a nucleic acid cargo (e.g. mRNA) as an input to the reaction. The schematic depicts an antigen-binding protein encapsulated by a micelle as a second input to the reaction. The schematic shows combinations of various insertion parameters considered, including, temperatures (ranging from 20° C. to 54° C.), incubation time (ranging from 30 in to 8 hours), mixing (ranging from 0 RPMs to 600 RPMs). The schematic illustrates that the reaction mix can then be subject to a cooling step for quenching the reaction. The reaction produces a targeted lipid nanoparticle that can be subject to tangential flow filtration and/or bulk filtration.

### EMBODIMENTS

EMBODIMENT 1. A process for the insertion of a targeting moiety into a lipid nanoparticle, the process comprising the steps of: inserting the targeting moiety into the lipid nanoparticle in a reaction that combines an amount of a targeting moiety with an amount of total lipids in a lipid nanoparticle for a time-period of at least one hour at a temperature no higher than 42° C., thereby forming an amount of targeted lipid nanoparticles; and quenching the insertion of the targeting moiety by cooling the reaction to about 20° C. or lower.

EMBODIMENT 2. The process of EMBODIMENT 1, whereby the process yields greater than 80% of targeted nanoparticles.

EMBODIMENT 3. The process of any one of EMBODIMENTS 1 and 2, whereby the inserting is conducted at a pH from about 6.2 to about 7.5.

EMBODIMENT 4. The process of any one of EMBODIMENTS 1-3, whereby the inserting is conducted at a pH of about 7.4.

EMBODIMENT 5. The process of any one of EMBODIMENTS 1-4, whereby the quenching of the insertion is performed by cooling the reaction to about 10° C. to about 20° C.

EMBODIMENT 6. The process of any one of EMBODIMENTS 1-5, whereby the quenching of the insertion is performed by cooling the reaction to about 10° C. to about 20° C.

EMBODIMENT 7. The process of any one of EMBODIMENTS 1-6, whereby the temperature is no higher than 32° C.

EMBODIMENT 8. The process of any one of EMBODIMENTS 1-7, whereby the time-period is a time-period from about one hour to about four hours at a temperature between 20° C. and 32° C.

EMBODIMENT 9. The process of any one of EMBODIMENTS 1-7, whereby the time-period is a time-period from about one hour to about two hours at a temperature between 20° C. and 32° C.

EMBODIMENT 10. The process of any one of EMBODIMENTS 1-7, whereby the time-period is a time-period from about one hour to about four hours at a temperature between 20° C. and 42° C.

EMBODIMENT 11. The process of any one of EMBODIMENTS 1-7, whereby the time-period is a time-period from about one hour to about two hours at a temperature between 20° C. and 42° C.

EMBODIMENT 12. The process of any one of EMBODIMENTS 1-7, whereby the time-period is a time-period of about two hours at a temperature of about 30° C.

EMBODIMENT 13. The process of any one of EMBODIMENTS 1-7, whereby the time-period is no more than four hours.

EMBODIMENT 14. The process of any one of EMBODIMENTS 1-13, whereby the insertion is performed on a 2-(N-morpholino)ethanesulfonic acid (MES) buffer or a tris(hydroxymethyl)aminomethane (Tris) or a HEPES (4-(2-hydroxyethyl)-1-piperazineethanesulfonic acid) (HEPES) buffer.

EMBODIMEN % 15. The process of any one of EMBODIMENTS 1-13, whereby the buffer is a 25 mM MES buffer comprising 150 mM NaCl at pH 6.5, 50 mM Tris buffer comprising 150 mM NaCl at pH 7.5, or 25 mM HEPES buffer comprising 150 mM NaCl at pH 7.4.

EMBODIMENT 16. The process of any one of EMBODIMENTS 1-15, wherein the targeting moiety is from about 10 kDA to about 160 kDA.

EMBODIMENT 17. The process of any one of EMBODIMENTS 1-17, wherein the targeting moiety is an antigen-binding protein.

EMBODIMENT 18. The process of any one of EMBODIMENTS 1-15, wherein the targeting moiety is a fragment antigen-binding (Fab).

EMBODIMENT 19. The process of any one of EMBODIMENTS 1-18, wherein the targeting moiety is a variable heavy domain of a heavy chain (VHH).

EMBODIMENT 20. The process of any one of EMBODIMENTS 1-19, wherein the targeting moiety is encapsulated in a polymeric micelle.

EMBODIMENT 21. The process of any one of EMBODIMENTS 1-20, wherein the polymeric micelle is a polyethylene glycol-micelle (PEG-micelle).

EMBODIMENT 22. The process of any one of EMBODIMENTS 1-14, wherein the lipid nanoparticle comprises at least one ionizable lipid.

EMBODIMENT 23. The process of EMBODIMENT 22, wherein the ionizable lipid is Lipid 1:

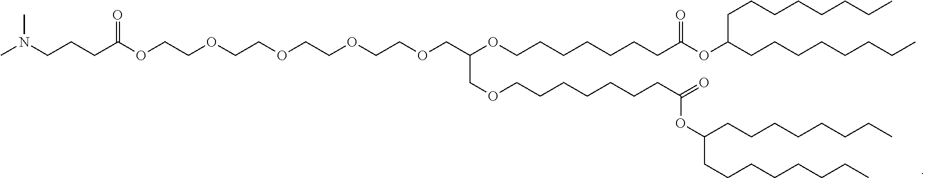 [SMILES: CCCCCCCCC(CCCCCCCC)OC(=O)CCCCCCCOCC(COCCOCCOCCOCCOC(=O)CCCN(C)C)OCCCCCCCC(=O)OC(CCCCCCCC)CCCCCCCC]

EMBODIMENT 24. The process of EMBODIMENT 22, wherein the ionizable lipid is Lipid 2:

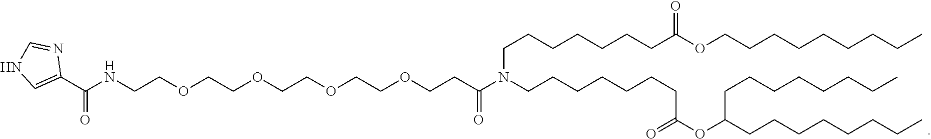 [SMILES: CCCCCCCCCOC(=O)CCCCCCCN(CCCCCCCC(=O)OC(CCCCCCCC)CCCCCCCC)C(=O)CCOCCOCCOCCOCCNC(=O)c1c[nH]cn1]

EMBODIMENT 25. The process of EMBODIMENT 22, wherein the ionizable lipid is Lipid 3:

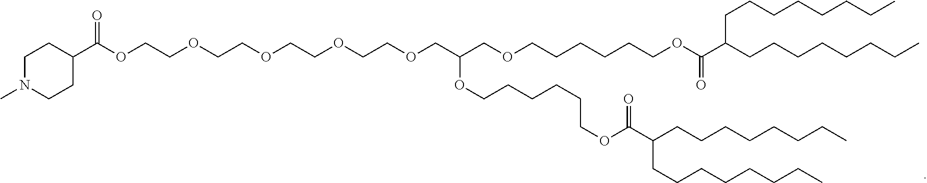 [SMILES: CCCCCCCCC(CCCCCCCC)C(=O)OCCCCCCOCC(COCCOCCOCCOCCOC(=O)C1CCN(C)CC1)OCCCCCCOC(=O)C(CCCCCCCC)CCCCCCCC]

EMBODIMENT 26. The process of EMBODIMENT 22, wherein the ionizable lipid is Lipid 4:

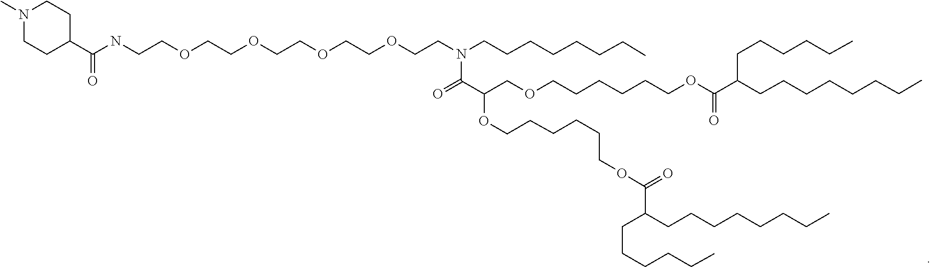

EMBODIMENT 27. The process of EMBODIMENT 22, wherein the ionizable lipid is Lipid 5:

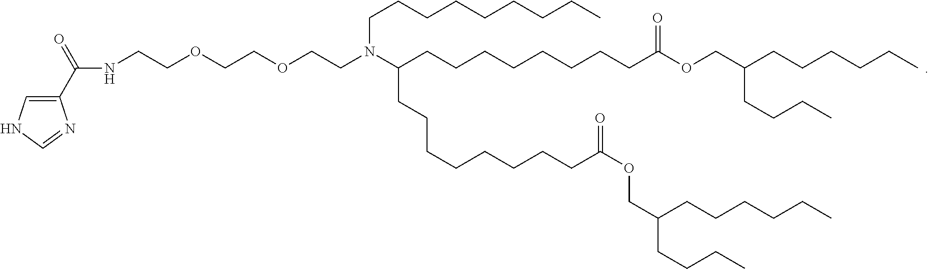 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC]

EMBODIMENT 28. The process of EMBODIMENT 22, wherein the ionizable lipid is Lipid 6:

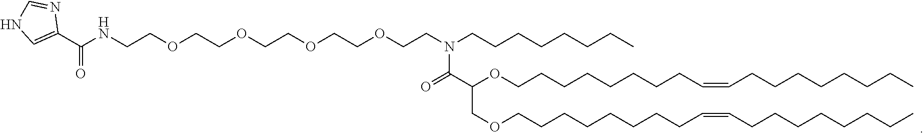 [SMILES: CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1]

EMBODIMENT 29. The process of EMBODIMENT 22, wherein the ionizable lipid is Lipid 7:

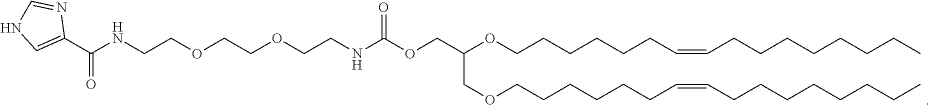 [SMILES: CCCCCCCC/C=C\CCCCCCOCC(COC(=O)NCCOCCOCCNC(=O)c1c[nH]cn1)OCCCCCC/C=C\CCCCCCCC]

EMBODIMENT 30. The process of EMBODIMENT 22, wherein the ionizable lipid is Lipid 8:

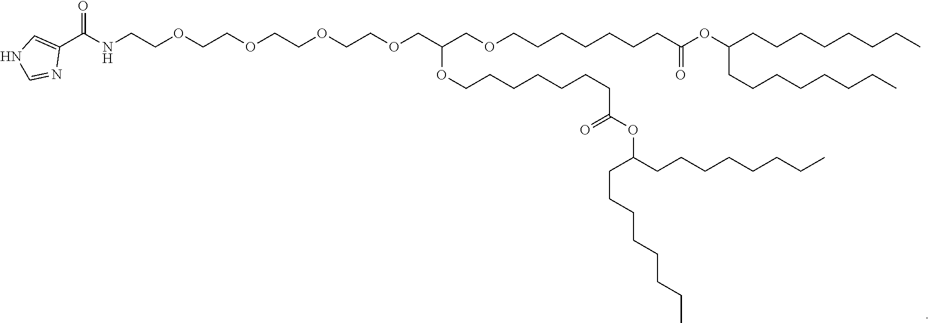 [SMILES: CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

EMBODIMENT 31. The process of EMBODIMENT 22, wherein the ionizable lipid is Lipid 9:

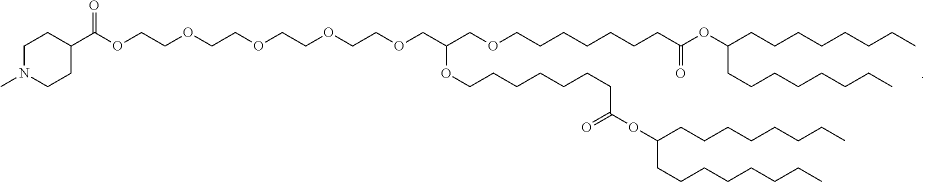 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC]

EMBODIMENT 32. The process of EMBODIMENT 22, wherein the ionizable lipid is Lipid 10:

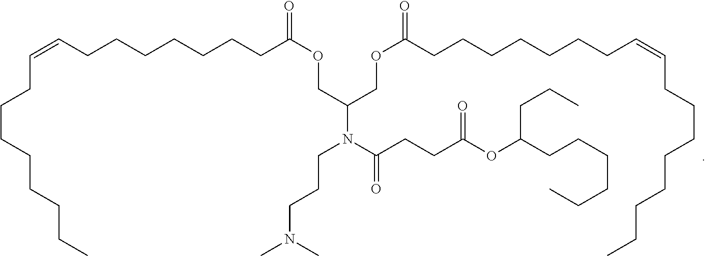 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

EMBODIMENT 33. The process of any one of EMBODIMENTS 1-32, whereby the inserting occurs at a rotational speed of zero revolutions per minute (0 RMPs) during the time-period.

EMBODIMENT 34. The process of any one of EMBODIMENTS 1-33, whereby the inserting occurs at a rotational speed of no more than three hundred revolutions per minute (300 RMPs) during the time-period.

EMBODIMENT 35. The process of any one of EMBODIMENTS 1-33, whereby the inserting occurs at a rotational speed of no more than six hundred revolutions per minute (600 RMPs) during the time-period.

EMBODIMENT 36. The process of any one of EMBODIMENTS 1-33, whereby the amount of the targeting moiety ranges from 0.15 μg of the targeting moiety per mol of the total lipids in the lipid nanoparticle to 30 g of the targeting moiety per mol of the total lipids in the lipid nanoparticle.

EMBODIMENT 37. The process of any one of EMBODIMENTS 1-38, whereby the lipid nanoparticle further comprises a nucleic acid cargo.

EMBODIMENT 38. The process of EMBODIMENT 37, whereby the amount of the targeting moiety is from 0.5× to 1.5× the amount of the nucleic acid cargo.

EMBODIMENT 39. The process of EMBODIMENT 37, whereby the amount of the targeting moiety is from 800 mg to 1,200 mg of targeting moiety per gram of the nucleic acid cargo.

EMBODIMENT 40. The process of any one of EMBODIMENTS 1-39, whereby the nucleic acid cargo is pre-encapsulated into the lipid nanoparticle prior to the insertion of the targeting moiety.

EMBODIMENT 41. The process of any one of EMBODIMENT 40, whereby insertion of the targeting moiety does not fragment the nucleic acid cargo.

EMBODIMENT 42. The process of any one of EMBODIMENT 41, whereby the nucleic acid cargo in the targeted lipid nanoparticle remains substantially unfragmented during at least 3 freeze thaw cycles.

EMBODIMENT 43. The process of any one of EMBODIMENTS 1-42, whereby greater than 50% of the nucleic acid cargo remains encapsulated in the targeted lipid nanoparticle during the reaction.

EMBODIMENT 44. The process of any one of EMBODIMENTS 1-33, wherein the lipid nanoparticle ranges from approximately 50 nanometers to 200 nanometers in size.

EMBODIMENT 45. The process of any one of EMBODIMENTS 1-44, wherein the lipid nanoparticle ranges from approximately 75 nanometers to 85 nanometers in size.

EMBODIMENT 46. The process of any one of EMBODIMENTS 1-45, wherein the lipid nanoparticle ranges from approximately 85 nanometers to 97 nanometers in size.

EMBODIMENT 47. The process of any one of EMBODIMENTS 1-46, wherein the lipid nanoparticle ranges from approximately 80 nanometers to 90 nanometers in size.

EMBODIMENT 48. The process of any one of EMBODIMENTS 1-47, whereby the insertion of the targeting moiety into the lipid nanoparticle increases the size of the lipid nanoparticle between 0% to 25%.

EMBODIMENT 49. The process of any one of EMBODIMENT 48, whereby the insertion of the targeting moiety into the lipid nanoparticle increases the size of the lipid nanoparticle by about 20%.

EMBODIMENT 50. The process of any one of EMBODIMENTS 1-49, whereby the targeted nanoparticle is stable for a time-period of at least 1 day at a temperature between 2° C. and 8° C.

EMBODIMENT 51. The process of any one of EMBODIMENTS 1-50, wherein the lipid nanoparticle comprises an amount of an ionizable lipid, an amount of cholesterol, an amount of neutral lipid, and an amount of a PEG-lipid.

EMBODIMENT 52. The process of EMBODIMENT 51, wherein the lipid nanoparticle comprises amounts of the ionizable lipid, the cholesterol, the neutral lipid, and the PEG-lipid at molar ratio ranges respectively of 40%-60% ionizable lipid, 20%-35% cholesterol, 10%-30% of neutral lipid, 0.5%-5% of PEG-lipid.

EMBODIMENT 53. The process of EMBODIMENT 51, wherein the ionizable lipid is selected from the group consisting of:

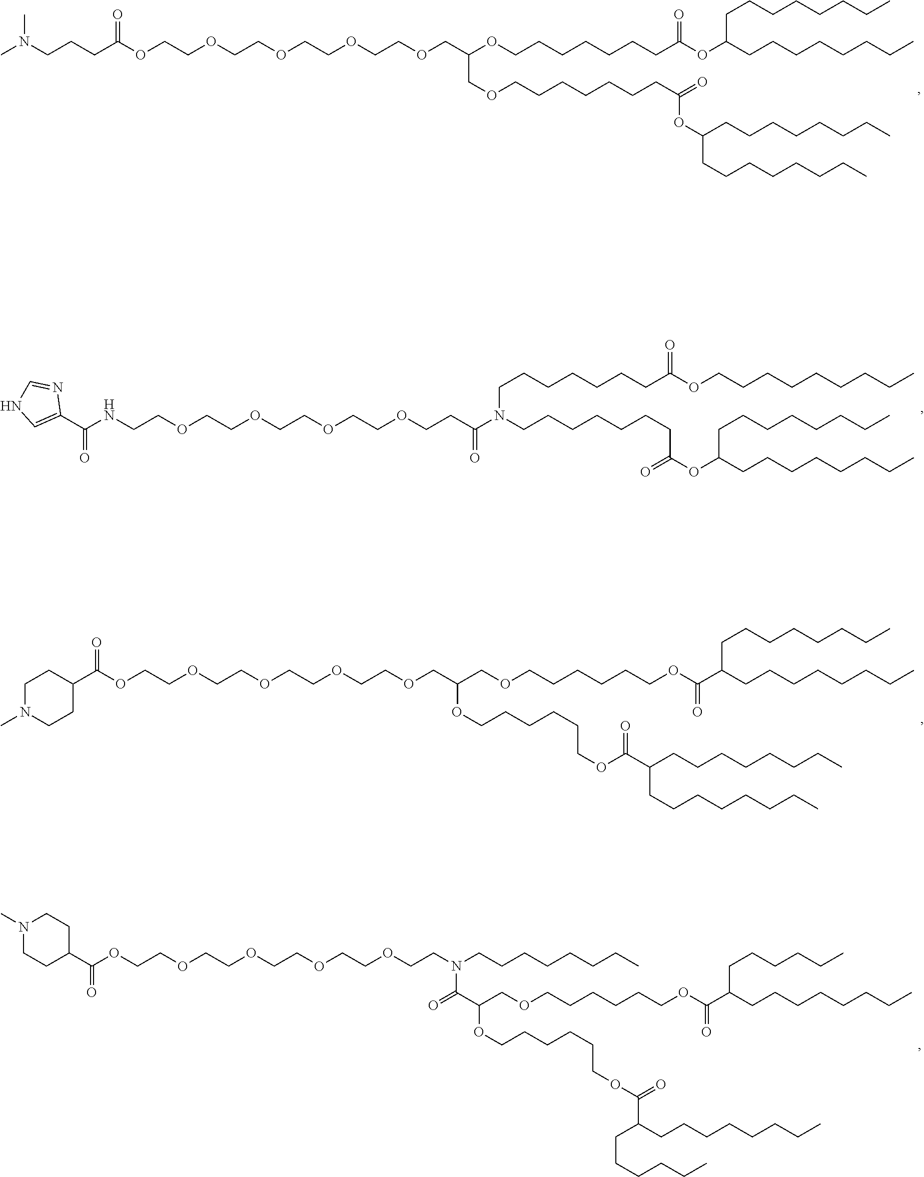

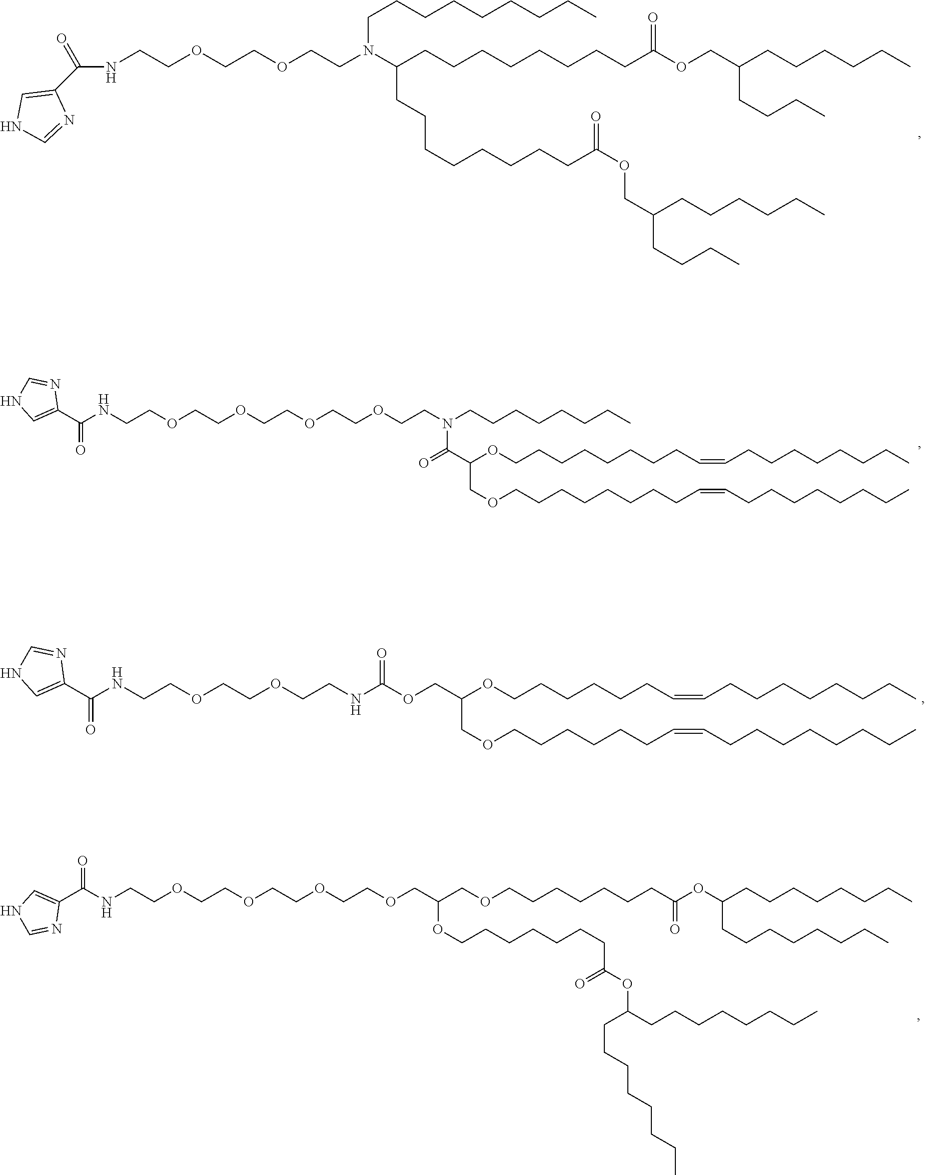 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC ; CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1 ; CCCCCCCC/C=C\CCCCCCOCC(COC(=O)NCCOCCOCCNC(=O)c1c[nH]cn1)OCCCCCC/C=C\CCCCCCCC ; CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

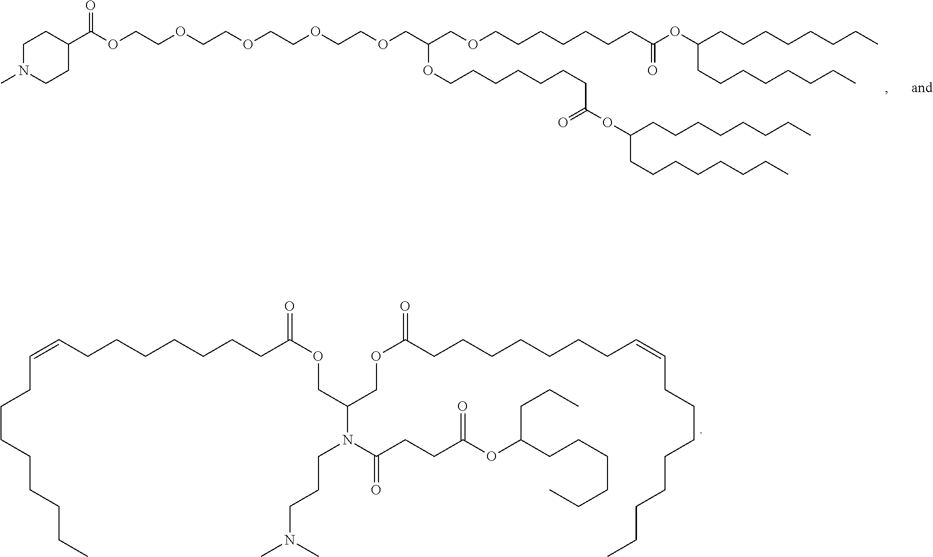 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC ; CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

EMBODIMENT 54. The process of EMBODIMENT 51, wherein the ionizable lipid is selected from the group consisting of a KC2 lipid, a DLin-MC2-DMA lipid, a DLin-MC3-DMA lipid, a DSDMA lipid, a DODMA lipid, a DLinDMA lipid, a DLenDMA lipid, a γ-DLenDMA lipid, a DLin-K-DMA lipid, a DLin-C2K-DMA lipid, a DLin-K-C3-DMA lipid, a DLin-K-C4-DMA lipid, a DLen-C2K-DMA lipid, a γ-DLen-C2K-DMA lipid, or a DLin-MP-DMA lipid.

EMBODIMENT 55. The process of EMBODIMENT 51, wherein the neutral lipid is a phosphatidylcholine lipid or a phosphatidylethanolamine lipid.

EMBODIMENT 56. The process of EMBODIMENT 55, wherein the phosphatidylcholine lipid or the phosphatidylethanolamine lipid is selected from the group consisting of DOPE, DSPC, DPPC, and POPC.

EMBODIMENT 57. A process for the insertion of a targeting moiety into a lipid nanoparticle, the process comprising the steps of: inserting the targeting moiety into the lipid nanoparticle in a reaction that combines at least 0.15 μg of the targeting moiety per gram of the total lipids in the lipid nanoparticle for a time-period of at least one hour at a temperature that does not exceed 54° C., thereby forming an amount of targeted nanoparticles; quenching the insertion of the targeting moiety by cooling the reaction to about 20° C. or lower.

EMBODIMENT 58. The process of EMBODIMENT 57, whereby the quenching of the insertion is performed by cooling the reaction to about 10° C. to about 20° C.

EMBODIMENT 59. The process of any one of EMBODIMENTS 57 or 58, whereby the quenching of the insertion is performed by cooling the reaction to about 15° C.

EMBODIMENT 60. The process of any one of EMBODIMENTS 57-59, whereby the temperature does not exceed 50° C.

EMBODIMENT 61. The process of any one of EMBODIMENTS 57-59, whereby the temperature does not exceed 42° C.

EMBODIMENT 62. The process of any one of EMBODIMENTS 57-59, whereby the temperature does not exceed 30° C.

EMBODIMENT 63. The process of any one of EMBODIMENTS 57-62, whereby the process yields greater than 80% of targeted nanoparticles.

EMBODIMENT 64. The process of any one of EMBODIMENTS 57-63, whereby the inserting is conducted at a pH from about 6.2 to about 7.5.

EMBODIMENT 65. The process of any one of EMBODIMENTS 57-64, whereby the inserting is conducted at a pH 7.4.

EMBODIMENT 66. The process of any one of EMBODIMENTS 57-52, whereby the time-period is for at least one to about four hours at the temperature that does not exceed 54° C.

EMBODIMENT 67. The process of any one of EMBODIMENTS 57-52, whereby the time-period is for at least one to about two hours at a temperature that does not exceed 54° C.

EMBODIMENT 68. The process of any one of EMBODIMENTS 57-52, whereby the time-period is for at least one to about four hours at the temperature that does not exceed 32° C.

EMBODIMENT 69. The process of any one of EMBODIMENTS 57-52, whereby the time-period is for at least one to about two hours at a temperature that does not exceed 32° C.

EMBODIMENT 70. The process of any one of EMBODIMENTS 57-69, whereby the time-period is a time-period of about two hours at a temperature of about 30° C.

EMBODIMENT 71. The process of EMBODIMENT 70, whereby the time-period is for no more than four hours.

EMBODIMENT 72. The process of any one of EMBODIMENTS 57-70, whereby the insertion is performed on a 2-(N-morpholino)ethanesulfonic acid (MES) buffer or a tris(hydroxymethyl)aminomethane (Tris) or a HEPES (4-(2-hydroxyethyl)-1-piperazineethanesulfonic acid) (HEPES) buffer.

EMBODIMENT 73. The process of any one of EMBODIMENTS 57-72, whereby the buffer is a 25 mM MES buffer comprising 150 mM NaCl at pH 6.5, 50 mM Tris buffer comprising 150 mM NaCl at pH 7.5, or 25 mM HEPES buffer comprising 150 mM NaCl at pH 7.4.

EMBODIMENT 74. The process of any one of EMBODIMENTS 57-73, wherein the targeting moiety is from about 10 kDA to about 160 kDA.

EMBODIMENT 75. The process of any one of EMBODIMENTS 57-74, wherein the targeting moiety is an antigen-binding protein.

EMBODIMENT 76. The process of any one of EMBODIMENTS 57-75, wherein the targeting moiety is a fragment antigen-binding (Fab).

EMBODIMENT 77. The process of any one of EMBODIMENTS 57-76, wherein the targeting moiety is a variable heavy domain of a heavy chain (VHH).

EMBODIMENT 78. The process of any one of EMBODIMENTS 57-77, wherein the targeting moiety is encapsulated in a polymeric micelle.

EMBODIMENT 79. The process of any one of EMBODIMENTS 57-78, wherein the polymeric micelle is a polyethylene glycol-micelle (PEG-micelle).

EMBODIMENT 80. The process of any one of EMBODIMENTS 57-79, wherein the lipid nanoparticle comprises at least one ionizable lipid.

EMBODIMENT 81. The process of EMBODIMENT 80, wherein the ionizable lipid is:

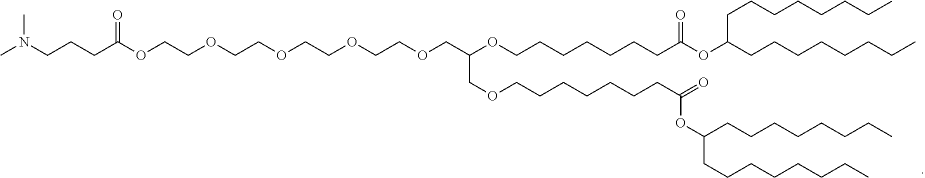 [SMILES: CCCCCCCCC(CCCCCCCC)OC(=O)CCCCCCCOCC(COCCOCCOCCOCCOC(=O)CCCN(C)C)OCCCCCCCC(=O)OC(CCCCCCCC)CCCCCCCC]

EMBODIMENT 82. The process of EMBODIMENT 80, wherein the ionizable lipid is:

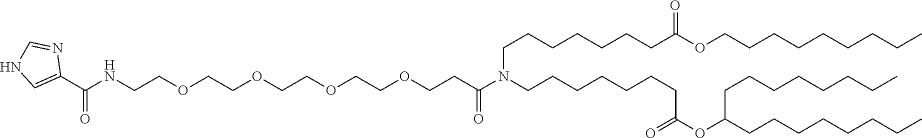 [SMILES: CCCCCCCCCOC(=O)CCCCCCCN(CCCCCCCC(=O)OC(CCCCCCCC)CCCCCCCC)C(=O)CCOCCOCCOCCOCCNC(=O)c1c[nH]cn1]

EMBODIMENT 83. The process of EMBODIMENT 80, wherein the ionizable lipid is:

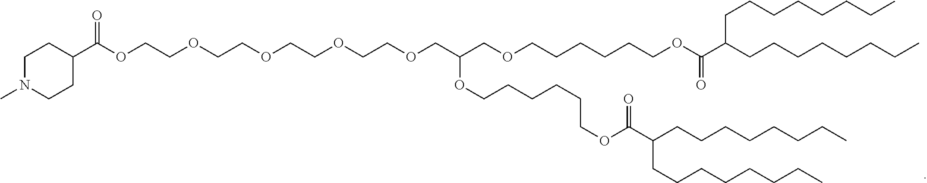 [SMILES: CCCCCCCCC(CCCCCCCC)C(=O)OCCCCCCOCC(COCCOCCOCCOCCOC(=O)C1CCN(C)CC1)OCCCCCCOC(=O)C(CCCCCCCC)CCCCCCCC]

EMBODIMENT 84. The process of EMBODIMENT 80, wherein the ionizable lipid is:

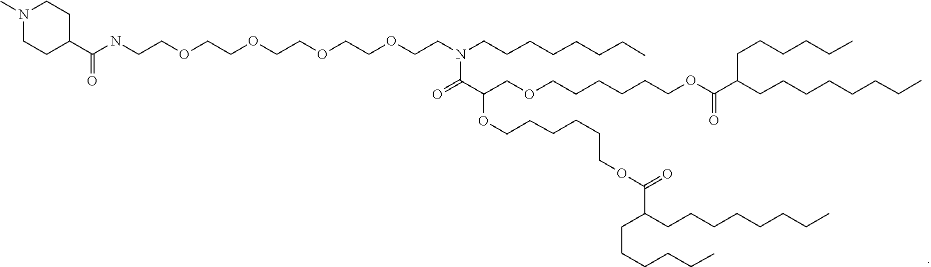

EMBODIMENT 85. The process of EMBODIMENT 80, wherein the ionizable lipid is:

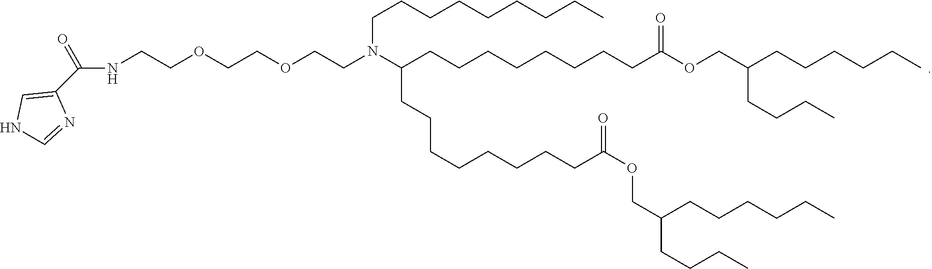 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC]

EMBODIMENT 86. The process of EMBODIMENT 80, wherein the ionizable lipid is:

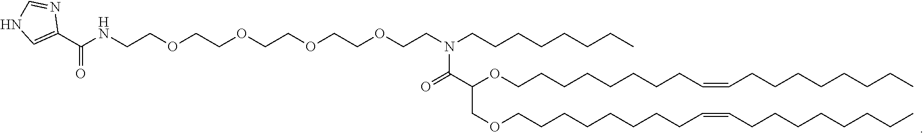 [SMILES: CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1]

EMBODIMENT 87. The process of EMBODIMENT 80, wherein the ionizable lipid is:

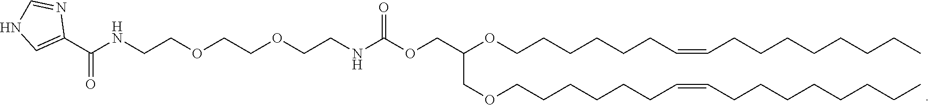 [SMILES: CCCCCCCC/C=C\CCCCCCOCC(COC(=O)NCCOCCOCCNC(=O)c1c[nH]cn1)OCCCCCC/C=C\CCCCCCCC]

EMBODIMENT 88. The process of EMBODIMENT 80, wherein the ionizable lipid is:

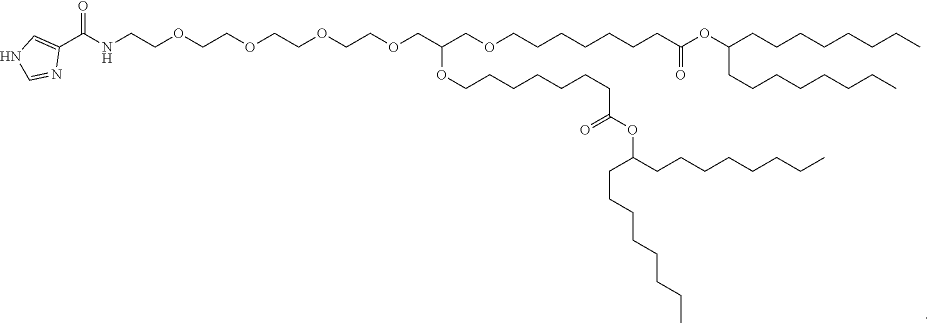 [SMILES: CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

EMBODIMENT 89. The process of EMBODIMENT 80, wherein the ionizable lipid is:

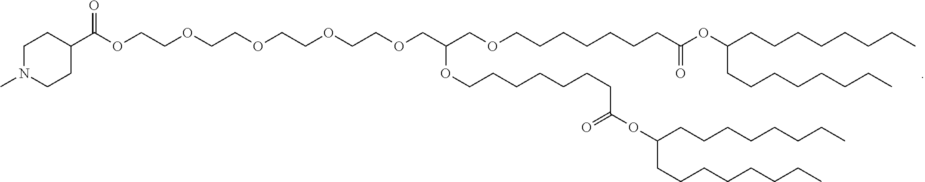 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC]

EMBODIMENT 90. The process of EMBODIMENT 80, wherein the ionizable lipid is:

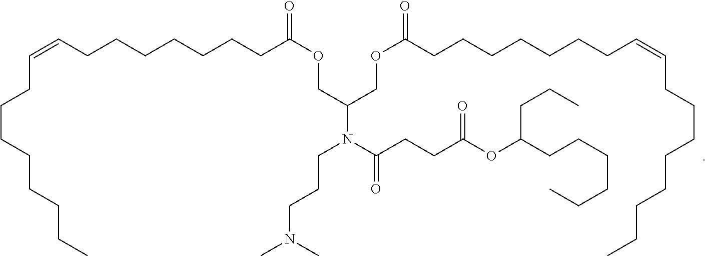 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

EMBODIMENT 91. The process of EMBODIMENT 80, wherein the ionizable lipid is a KC2 lipid.

EMBODIMENT 92. The process of any one of EMBODIMENTS 57-91, whereby the inserting occurs at a rotational speed of zero revolutions per minute (0 RMPs) during the time-period.

EMBODIMENT 93. The process of any one of EMBODIMENTS 57-92, whereby the inserting occurs at a rotational speed of no more than three hundred revolutions per minute (300 RMPs) during the time-period.

EMBODIMENT 94. The process of any one of EMBODIMENTS 57-93, whereby the inserting occurs at a rotational speed of no more than six hundred revolutions per minute (600 RMPs) during the time-period.

EMBODIMENT 95. The process of any one of EMBODIMENTS 57-94, whereby the amount of the targeting moiety ranges from 0.15 μg of the targeting moiety per mol of the total lipids in the lipid nanoparticle to 30 g of the targeting moiety per mol of the total lipids in the lipid nanoparticle.

EMBODIMENT 96. The process of any one of EMBODIMENTS 57-97, whereby the lipid nanoparticle further comprises a nucleic acid cargo.

EMBODIMENT 97. The process of EMBODIMENT 96, whereby the amount of the targeting moiety is from 0.5× to 1.5× the amount of the nucleic acid cargo.

EMBODIMENT 98. The process of EMBODIMENT 96, whereby the amount of the targeting moiety is from 800 mg to 1,200 mg of targeting moiety per gram of the nucleic acid cargo.

EMBODIMENT 99. The process of EMBODIMENT 98, wherein the nucleic acid cargo is an mRNA encoding a variable domain of heavy chain antibody (VHH) moiety or a fragment binding antigen (Fab).

EMBODIMENT 100. The process of any one of EMBODIMENTS 57-99, whereby the nucleic acid cargo is pre-encapsulated into the lipid nanoparticle prior to the insertion of the targeting moiety.

EMBODIMENT 101. The process of EMBODIMENT 100, whereby insertion of the targeting moiety does not fragment the nucleic acid cargo.

EMBODIMENT 102. The process of EMBODIMENT 100, whereby the nucleic acid cargo in the targeted lipid nanoparticle remains substantially unfragmented during at least 3 freeze thaw cycles.

EMBODIMENT 103. The process of any one of EMBODIMENTS 57-102, whereby greater than 50% of the nucleic acid cargo remains encapsulated in the targeted lipid nanoparticle during the reaction.

EMBODIMENT 104. The process of any one of EMBODIMENTS 57-103, wherein the lipid nanoparticle ranges from approximately 50 nanometers to 200 nanometers in size.

EMBODIMENT 105. The process of any one of EMBODIMENTS 57-103, wherein the lipid nanoparticle ranges from approximately 75 nanometers to 85 nanometers in size.

EMBODIMENT 106. The process of any one of EMBODIMENTS 57-103, wherein the lipid nanoparticle ranges from approximately 85 nanometers to 97 nanometers in size.

EMBODIMENT 107. The process of any one of EMBODIMENTS 57-103, wherein the lipid nanoparticle ranges from approximately 80 nanometers to 90 nanometers in size.

EMBODIMENT 108. The process of any one of EMBODIMENTS 57-107, whereby the insertion of the targeting moiety into the lipid nanoparticle increases the size of the lipid nanoparticle between 0% to 25%.

EMBODIMENT 109. The process of any one of EMBODIMENTS 57-108, whereby the insertion of the targeting moiety into the lipid nanoparticle increases the size of the lipid nanoparticle by about 20%.

EMBODIMENT 110. The process of any one of EMBODIMENTS 57-109, whereby the targeted nanoparticle is stable for a time-period of at least 1 day at a temperature between 2° C. and 8° C.

EMBODIMENT 111. The process of any one of EMBODIMENTS 57-110, wherein the lipid nanoparticle comprises an amount of an ionizable lipid, an amount of cholesterol, an amount of neutral lipid, and an amount of a PEG-lipid.

EMBODIMENT 112. The process of EMBODIMENT 111, wherein the lipid nanoparticle comprises amounts of the ionizable lipid, the cholesterol, the neutral lipid, and the PEG-lipid at molar ratio ranges respectively of 40%-60% ionizable lipid, 20%-35% cholesterol, 10%-30% of neutral lipid, 0.5%-5% of PEG-lipid.

EMBODIMENT 113. The process of EMBODIMENT 111, wherein the ionizable lipid is selected from the group consisting of a Lipid 1, a Lipid 2, a Lipid 3, a Lipid 4, a Lipid 5, a Lipid 6, a Lipid 7, a Lipid 8, a Lipid 9, or a Lipid 10 ionizable lipid.

EMBODIMENT 114. The process of EMBODIMENT 111, wherein the ionizable lipid is selected from the group consisting of a KC2 lipid, a DLin-MC2-DMA lipid, a DLin-MC3-DMA lipid, a DSDMA lipid, a DODMA lipid, a DLinDMA lipid, a DLenDMA lipid, a γ-DLenDMA lipid, a DLin-K-DMA lipid, a DLin-C2K-DMA lipid, a DLin-K-C3-DMA lipid, a DLin-K-C4-DMA lipid, a DLen-C2K-DMA lipid, a γ-DLen-C2K-DMA lipid, or a DLin-MP-DMA lipid.

EMBODIMENT 115. The process of EMBODIMENT 111, wherein the neutral lipid is a phosphatidylcholine lipid or a phosphatidylethanolamine lipid.

EMBODIMENT 116. The process of EMBODIMENT 115, wherein the phosphatidylcholine lipid or the phosphatidylethanolamine lipid is selected from the group consisting of DOPE, DSPC, DPPC, and POPC.

EMBODIMENT 117. A process for the insertion of a targeting moiety into a lipid nanoparticle, the process comprising the steps of: inserting from 0.15 μg to 30 g of the targeting moiety into the lipid nanoparticle in a soluble reaction by combining from 0.15 μg of the targeting moiety per gram of the total lipid nanoparticle to 30 g of the targeting moiety per gram of the total lipid nanoparticle for a time-period of at least one hour at a temperature that does not exceed 54° C., whereby the soluble reaction produces a targeted nanoparticle comprising from 0.15 μg to 30 mg of the targeted moiety incorporated into its outer membrane.

EMBODIMENT 118. The process of EMBODIMENT 117, whereby the amount of the targeting moiety ranges from 0.15 μg of the targeting moiety per mol of the total lipids in the lipid nanoparticle to 15 g of the targeting moiety per mol of the total lipids in the lipid nanoparticle.

EMBODIMENT 119. The process of EMBODIMENT 117, whereby the amount of the targeting moiety ranges from 0.15 μg of the targeting moiety per mol of the total lipids in the lipid nanoparticle to 5 g of the targeting moiety per mol of the total lipids in the lipid nanoparticle.

EMBODIMENT 120. The process of any one of EMBODIMENTS 117-119, whereby the process produces a targeted nanoparticle comprising from 0.15 μg to 25 mg, from 0.15 μg to 20 mg, from 0.15 μg to 15 mg, from 0.15 μg to 10 mg, from 0.15 μg to 5 mg, from 0.15 μg to 1 mg of the targeted moiety incorporated into its outer membrane.

EMBODIMENT 121. The process of any one of EMBODIMENTS 117-121, whereby the process yields greater than 80% of targeted nanoparticles.

EMBODIMENT 122. The process of any one of EMBODIMENTS 117-121, whereby the inserting is conducted at a pH from about 6.2 to about 7.5.

EMBODIMENT 123. The process of any one of EMBODIMENTS 117-121, whereby the inserting is conducted at a pH from about 7.4.

EMBODIMENT 124. The process of any one of EMBODIMENTS 117-123, whereby the time-period is a time-period from about one hour to about four hours at a temperature between 20° C. and 42° C.

EMBODIMENT 125. The process of any one of EMBODIMENTS 117-123, whereby the time-period is a time-period from about one hour to about two hours at a temperature between 20° C. and 42° C.

EMBODIMENT 126. The process of any one of EMBODIMENTS 117-123, whereby the time-period is a time-period from about one hour to about four hours at a temperature between 20° C. and 32° C.

EMBODIMENT 127. The process of any one of EMBODIMENTS 117-123, whereby the time-period is a time-period from about one hour to about two hours at a temperature between 20° C. and 32° C.

EMBODIMENT 128. The process of any one of EMBODIMENTS 117-123, whereby the time-period is a time-period of about two hours at a temperature of about 30° C.

EMBODIMENT 129. The process of any one of EMBODIMENTS 117-123, whereby the time-period is no more than four hours.

EMBODIMENT 130. The process of any one of EMBODIMENTS 117-129, whereby the insertion is performed on a 2-(N-morpholino)ethanesulfonic acid (MES) buffer or a tris(hydroxymethyl)aminomethane (Tris), or a HEPES (4-(2-hydroxyethyl)-1-piperazineethanesulfonic acid) (HEPES) buffer.

EMBODIMENT 131. The process of any one of EMBODIMENTS 117-130, whereby the buffer is a 25 mM MES buffer comprising 150 mM NaCl at pH 6.5, 50 mM Tris buffer comprising 150 mM NaCl at pH 7.5, or 25 mM HEPES buffer comprising 150 mM NaCl at pH 7.4.

EMBODIMENT 132. The process of any one of EMBODIMENTS 117-131, wherein the targeting moiety is from about 10 kDA to about 160 kDA.

EMBODIMENT 133. The process of any one of EMBODIMENTS 117-131, wherein the targeting moiety is an antigen-binding protein.

EMBODIMENT 134. The process of any one of EMBODIMENTS 117-131, wherein the targeting moiety is a fragment antigen-binding (Fab).

EMBODIMENT 135. The process of any one of EMBODIMENTS 117-131, wherein the targeting moiety is a variable heavy domain (VHH).

EMBODIMENT 136. The process of any one of EMBODIMENTS 117-135, wherein the targeting moiety is encapsulated in a polymeric micelle.

EMBODIMENT 137. The process of EMBODIMENT 136, wherein the polymeric micelle is a polyethylene glycol-micelle (PEG-micelle).

EMBODIMENT 138. The process of any one of EMBODIMENTS 117-137, wherein the lipid nanoparticle comprises at least one ionizable lipid.

EMBODIMENT 139. The process of EMBODIMENT 138, wherein the ionizable lipid is

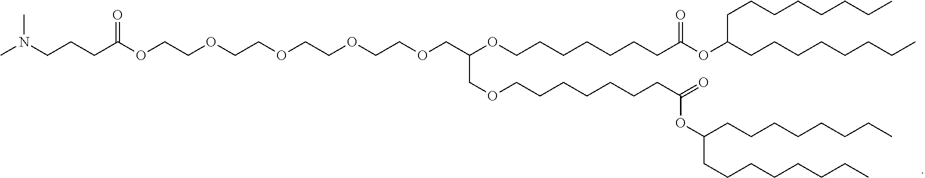 [SMILES: CCCCCCCCC(CCCCCCCC)OC(=O)CCCCCCCOCC(COCCOCCOCCOCCOC(=O)CCCN(C)C)OCCCCCCCC(=O)OC(CCCCCCCC)CCCCCCCC]

EMBODIMENT 140. The process of EMBODIMENT 138, wherein the ionizable lipid is

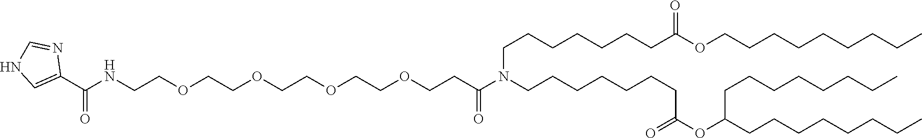 [SMILES: CCCCCCCCCOC(=O)CCCCCCCN(CCCCCCCC(=O)OC(CCCCCCCC)CCCCCCCC)C(=O)CCOCCOCCOCCOCCNC(=O)c1c[nH]cn1]

EMBODIMENT 141. The process of EMBODIMENT 138, wherein the ionizable lipid is

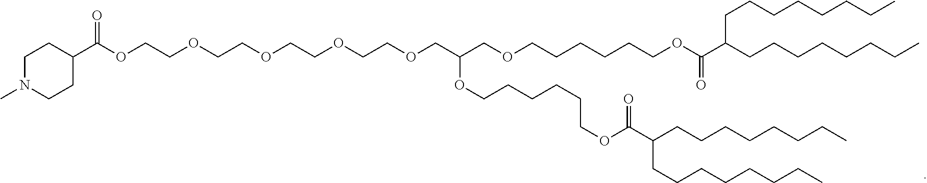 [SMILES: CCCCCCCCC(CCCCCCCC)C(=O)OCCCCCCOCC(COCCOCCOCCOCCOC(=O)C1CCN(C)CC1)OCCCCCCOC(=O)C(CCCCCCCC)CCCCCCCC]

EMBODIMENT 142. The process of EMBODIMENT 138, wherein the ionizable lipid is

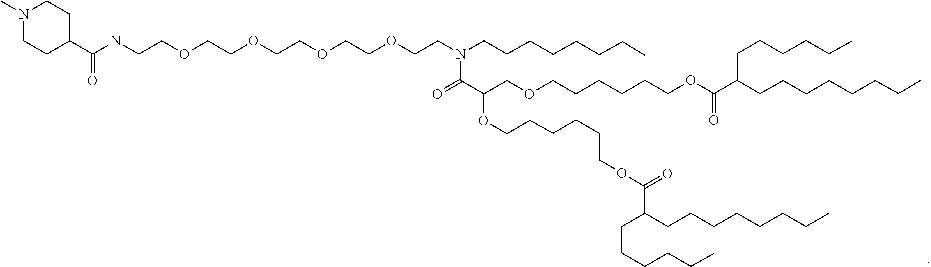

EMBODIMENT 143. The process of EMBODIMENT 138, wherein the ionizable lipid is

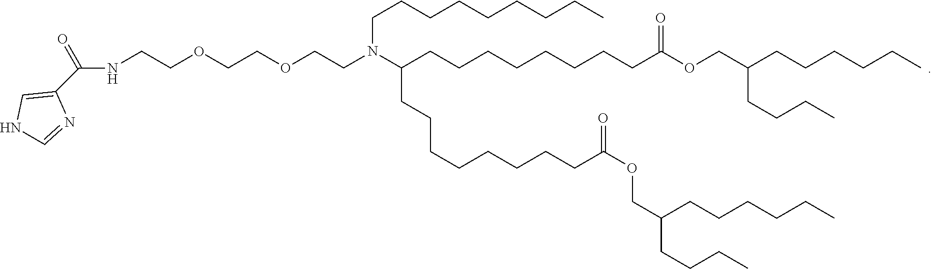 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC]

EMBODIMENT 144. The process of EMBODIMENT 138, wherein the ionizable lipid is

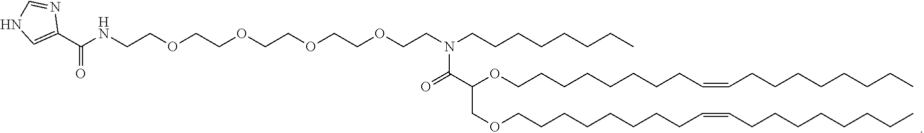 [SMILES: CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1]

EMBODIMENT 145. The process of EMBODIMENT 138, wherein the ionizable lipid is

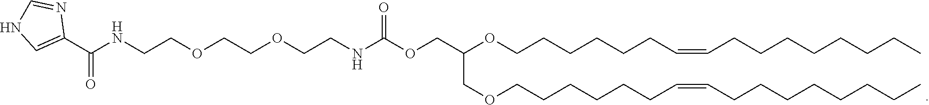 [SMILES: CCCCCCCC/C=C\CCCCCCOCC(COC(=O)NCCOCCOCCNC(=O)c1c[nH]cn1)OCCCCCC/C=C\CCCCCCCC]

EMBODIMENT 146. The process of EMBODIMENT 138, wherein the ionizable lipid is

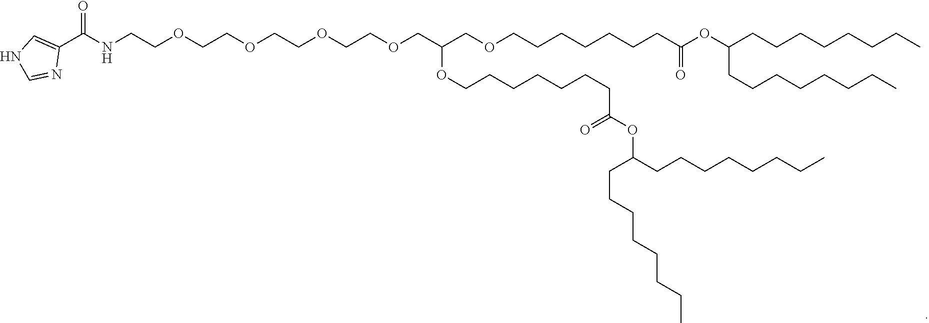 [SMILES: CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

EMBODIMENT 147. The process of EMBODIMENT 138, wherein the ionizable lipid is

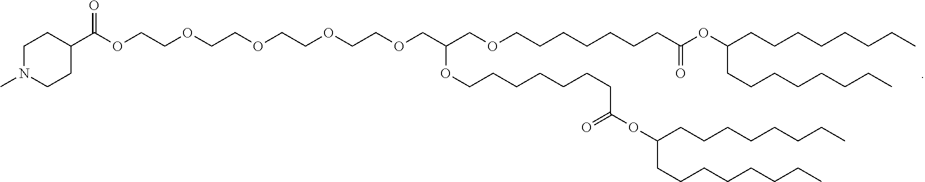 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC]

EMBODIMENT 148. The process of EMBODIMENT 138, wherein the ionizable lipid is

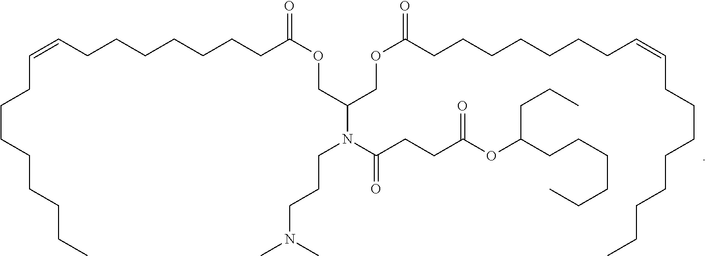 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

EMBODIMENT 149. The process of any one of EMBODIMENTS 117-148, whereby the inserting occurs at a rotational speed of zero revolutions per minute (0 RMPs) during the time-period.

EMBODIMENT 150. The process of any one of EMBODIMENTS 117-149, whereby the inserting occurs at a rotational speed of no more than three hundred revolutions per minute (300 RMPs) during the time-period.

EMBODIMENT 151. The process of any one of EMBODIMENTS 117-150, whereby the inserting occurs at a rotational speed of no more than six hundred revolutions per minute (600 RMPs) during the time-period.

EMBODIMENT 152. The process of any one of EMBODIMENTS 117-151, whereby the lipid nanoparticle further comprises a nucleic acid cargo.

EMBODIMENT 153. The process of any one of EMBODIMENTS 117-152, wherein the nucleic acid cargo is an mRNA encoding a VHH antigen binding moiety or a fragment antigen-binding (Fab) moiety.

EMBODIMENT 154. The process of any one of EMBODIMENTS 117-153, whereby the nucleic acid cargo is pre-encapsulated into the lipid nanoparticle prior to the insertion of the targeting moiety.

EMBODIMENT 155. The process of any one of EMBODIMENTS 117-154, whereby insertion of the targeting moiety does not fragment the nucleic acid cargo.

EMBODIMENT 156. The process of any one of EMBODIMENTS 117-154, whereby the nucleic acid cargo in the targeted lipid nanoparticle remains substantially unfragmented during at least 3 freeze thaw cycles.

EMBODIMENT 157. The process of any one of EMBODIMENTS 117-156, whereby greater than 50% of the nucleic acid cargo remains encapsulated in the targeted lipid nanoparticle during the reaction.

EMBODIMENT 158. The process of any one of EMBODIMENTS 117-157, wherein the lipid nanoparticle ranges from approximately 50 nanometers to 200 nanometers in size.

EMBODIMENT 159. The process of any one of EMBODIMENTS 117-158, wherein the lipid nanoparticle ranges from approximately 75 nanometers to 85 nanometers in size.

EMBODIMENT 160. The process of any one of EMBODIMENTS 117-159, wherein the lipid nanoparticle ranges from approximately 85 nanometers to 97 nanometers in size.

EMBODIMENT 161. The process of any one of EMBODIMENTS 117-160, wherein the lipid nanoparticle ranges from approximately 80 nanometers to 90 nanometers in size.

EMBODIMENT 162. The process of any one of EMBODIMENTS 117-161, whereby the insertion of the targeting moiety into the lipid nanoparticle increases the size of the lipid nanoparticle between 0% to 25%.

EMBODIMENT 163. The process of any one of EMBODIMENTS 117-162, whereby the insertion of the targeting moiety into the lipid nanoparticle increases the size of the lipid nanoparticle by about 20%.

EMBODIMENT 164. The process of any one of EMBODIMENTS 117-163, whereby the targeted nanoparticle is stable for a time-period of at least 1 day at a temperature between 2° C. and 8° C.

EMBODIMENT 165. The process of any one of EMBODIMENTS 117-164, wherein the lipid nanoparticle comprises an amount of an ionizable lipid, an amount of cholesterol, an amount of neutral lipid, and an amount of a PEG-lipid.

EMBODIMENT 166. The process of EMBODIMENT 165, wherein the lipid nanoparticle comprises amounts of the ionizable lipid, the cholesterol, the neutral lipid, and the PEG-lipid at molar ratio ranges respectively of 40%-60% ionizable lipid, 20%-35% cholesterol, 10%-30% of neutral lipid, 0.5%-5% of PEG-lipid.

EMBODIMENT 167. The process of EMBODIMENT 165, wherein the ionizable lipid is selected from the group consisting of a Lipid 1, lipid 2, Lipid 3, Lipid 4, Lipid 5, Lipid 6, Lipid 7, Lipid 8, Lipid 9, Lipid 10, a KC2 lipid, a DLin-MC2-DMA lipid, a DLin-MC3-DMA lipid, a DSDMA lipid, a DODMA lipid, a DLinDMA lipid, a DLenDMA lipid, a γ-DLenDMA lipid, a DLin-K-DMA lipid, a DLin-C2K-DMA lipid, a DLin-K-C3-DMA lipid, a DLin-K-C4-DMA lipid, a DLen-C2K-DMA lipid, a γ-DLen-C2K-DMA lipid, or a DLin-MP-DMA lipid.

EMBODIMENT 168. The process of EMBODIMENT 165, wherein the neutral lipid is a phosphatidylcholine lipid or a phosphatidylethanolamine lipid.

EMBODIMENT 169. The process of EMBODIMENT 165, wherein the phosphatidylcholine lipid or the phosphatidylethanolamine lipid is selected from the group consisting of DOPE, DSPC, DPPC, and POPC.

EMBODIMENT 170. A targeted lipid nanoparticle produced by a process comprising the steps of: combining in a soluble reaction from 0.15 μg of the targeting moiety per gram of the total lipid nanoparticle to 30 g of the targeting moiety per gram of the total lipid nanoparticle for a time-period of at least one hour at in a lipid nanoparticle having a nucleic acid cargo encapsulated therein, whereby the soluble reaction is conducted for a time-period of at least one hour at a temperature no higher than 42° C., thereby producing an amount of targeted lipid nanoparticles whereby the nucleic acid cargo within the targeted lipid nanoparticles is less than 20% degraded compared to a nucleic acid cargo in a particle where the targeting moiety is not inserted by the process.

EMBODIMENT 171. The targeted lipid nanoparticle of EMBODIMENT 170, whereby the nucleic acid cargo within the targeted lipid nanoparticles is less than 10% degraded compared to a nucleic acid cargo in a particle where the targeting moiety is not inserted by the process.

EMBODIMENT 172. The targeted lipid nanoparticle of any one of EMBODIMENTS 170 and 171, whereby the process yields greater than 80% of targeted lipid nanoparticles.

EMBODIMENT 173. The targeted lipid nanoparticle of any one of EMBODIMENTS 170-172, whereby the inserting is conducted at a pH between 6.2 and 7.5.

EMBODIMENT 174. The targeted lipid nanoparticle of any one of EMBODIMENTS 170-172, whereby the inserting is conducted at a pH of about 7.4.

EMBODIMENT 175. The targeted lipid nanoparticle of any one of EMBODIMENTS 170-174, whereby the time-period is a time-period between one hour to four hours at a temperature between 20° C. and 42° C.

EMBODIMENT 176. The targeted lipid nanoparticle of any one of EMBODIMENTS 170-174, whereby the time-period is a time-period between one hour to two hours at a temperature between 20° C. and 42° C.

EMBODIMENT 177. The targeted lipid nanoparticle of any one of EMBODIMENTS 170-174, whereby the time-period is a time-period between one hour to four hours at a temperature between 20° C. and 32° C.

EMBODIMENT 178. The targeted lipid nanoparticle of any one of EMBODIMENTS 170-174, whereby the time-period is a time-period between one hour to two hours at a temperature between 20° C. and 32° C.

EMBODIMENT 179. The targeted lipid nanoparticle of any one of EMBODIMENTS 170-178, whereby the time-period is a time-period of about two hours at a temperature of about 30° C.

EMBODIMENT 180. The targeted lipid nanoparticle of any one of EMBODIMENTS 170-172, whereby the time-period is no more than four hours.

EMBODIMENT 181. The targeted lipid nanoparticle of any one of EMBODIMENTS 170-180, whereby the insertion is performed on a 2-(N-morpholino)ethanesulfonic acid (MES) buffer.

EMBODIMENT 182. The targeted lipid nanoparticle of any one of EMBODIMENTS 170-180, whereby the buffer is a 25 mM MES buffer comprising 150 mM NaCl at pH 6.5 or a tris(hydroxymethyl)aminomethane (Tris) or a HEPES (4-(2-hydroxyethyl)-1-piperazineethanesulfonic acid) (HEPES) buffer.

EMBODIMENT 183. The targeted lipid nanoparticle of any one of EMBODIMENTS 170-182, whereby wherein the targeting moiety is about 10 kDA to about 160 kDA.

EMBODIMENT 184. The targeted lipid nanoparticle of any one of EMBODIMENTS 170-182, whereby wherein the targeting moiety is an antigen-binding protein.

EMBODIMENT 185. The targeted lipid nanoparticle of any one of EMBODIMENTS 170-182, whereby wherein the targeting moiety is a fragment antigen-binding (Fab).

EMBODIMENT 186. The targeted lipid nanoparticle of any one of EMBODIMENTS 170-186, wherein the targeting moiety is encapsulated in a polymeric micelle.

EMBODIMENT 187. The targeted lipid nanoparticle of EMBODIMENT 185, wherein the polymeric micelle is a polyethylene glycol-micelle (PEG-micelle).

EMBODIMENT 188. The targeted lipid nanoparticle of any one of EMBODIMENTS 170-186, wherein the nucleic acid cargo encodes an antigen binding VHH moiety.

EMBODIMENT 189. The targeted lipid nanoparticle of any one of EMBODIMENTS 170-188, wherein the lipid nanoparticle comprises at least one ionizable lipid.

EMBODIMENT 190. The targeted lipid nanoparticle of EMBODIMENT 189, wherein the ionizable lipid is

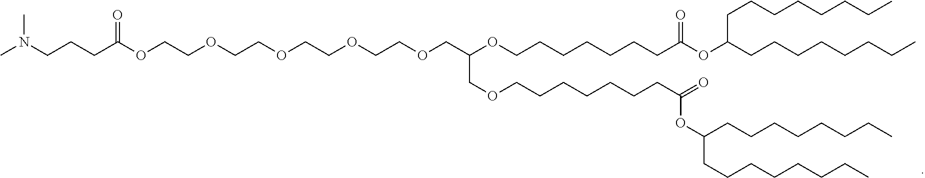 [SMILES: CCCCCCCCC(CCCCCCCC)OC(=O)CCCCCCCOCC(COCCOCCOCCOCCOC(=O)CCCN(C)C)OCCCCCCCC(=O)OC(CCCCCCCC)CCCCCCCC]

EMBODIMENT 191. The targeted lipid nanoparticle of EMBODIMENT 189, wherein the ionizable lipid is

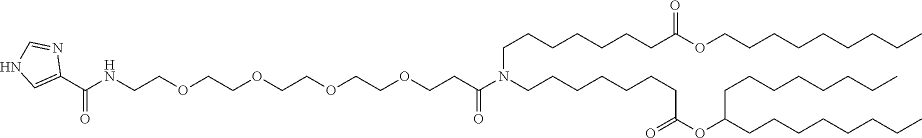 [SMILES: CCCCCCCCCOC(=O)CCCCCCCN(CCCCCCCC(=O)OC(CCCCCCCC)CCCCCCCC)C(=O)CCOCCOCCOCCOCCNC(=O)c1c[nH]cn1]

EMBODIMENT 192. The targeted lipid nanoparticle of EMBODIMENT 189, wherein the ionizable lipid is

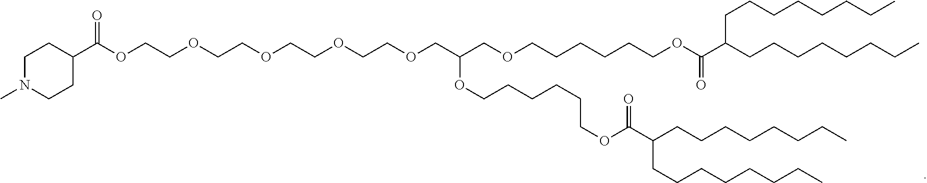 [SMILES: CCCCCCCCC(CCCCCCCC)C(=O)OCCCCCCOCC(COCCOCCOCCOCCOC(=O)C1CCN(C)CC1)OCCCCCCOC(=O)C(CCCCCCCC)CCCCCCCC]

EMBODIMENT 193. The targeted lipid nanoparticle of EMBODIMENT 189, wherein the ionizable lipid is

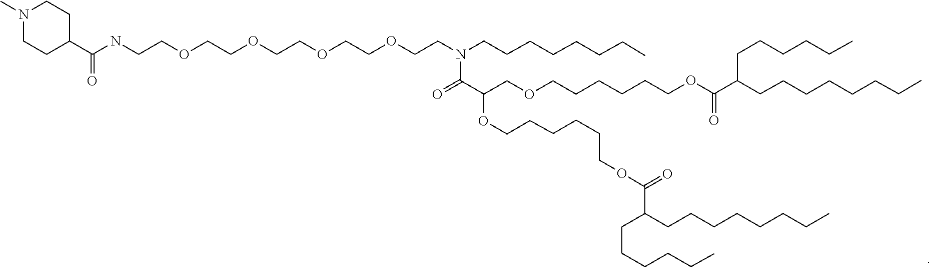

EMBODIMENT 194. The targeted lipid nanoparticle of EMBODIMENT 189, wherein the ionizable lipid is

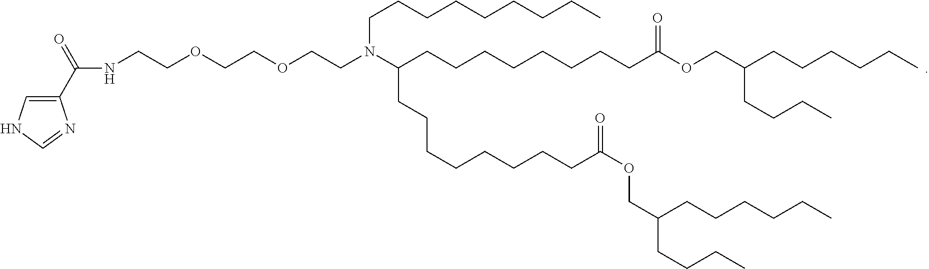 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC]

EMBODIMENT 195. The targeted lipid nanoparticle of EMBODIMENT 189, wherein the ionizable lipid is

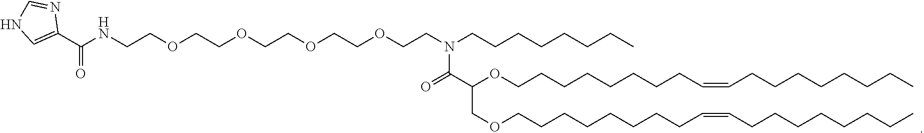 [SMILES: CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1]

EMBODIMENT 196. The targeted lipid nanoparticle of EMBODIMENT 189, wherein the ionizable lipid is

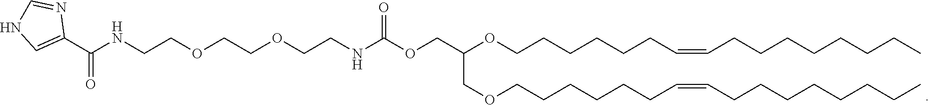 [SMILES: CCCCCCCC/C=C\CCCCCCOCC(COC(=O)NCCOCCOCCNC(=O)c1c[nH]cn1)OCCCCCC/C=C\CCCCCCCC]

EMBODIMENT 197. The targeted lipid nanoparticle of EMBODIMENT 189, wherein the ionizable lipid is

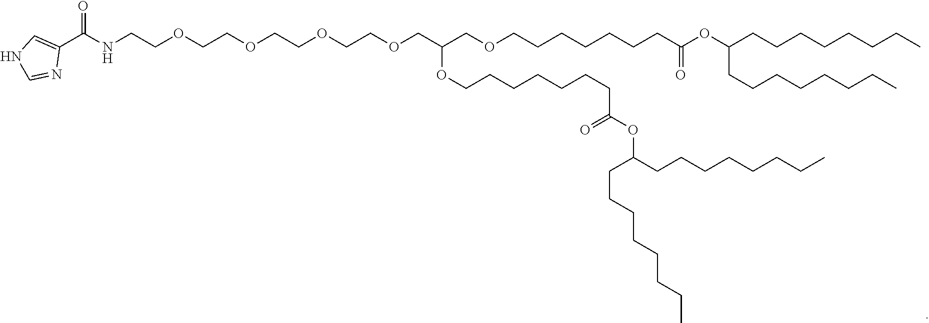 [SMILES: CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

EMBODIMENT 198. The targeted lipid nanoparticle of EMBODIMENT 189, wherein the ionizable lipid is

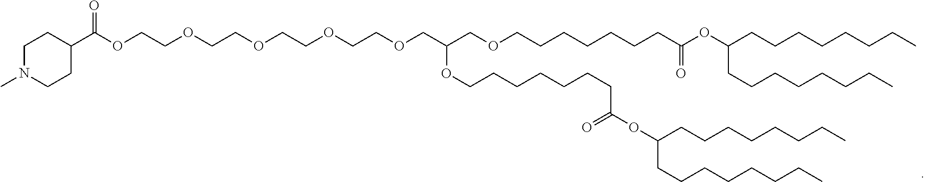 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC]

EMBODIMENT 199. The targeted lipid nanoparticle of EMBODIMENT 189, wherein the ionizable lipid is

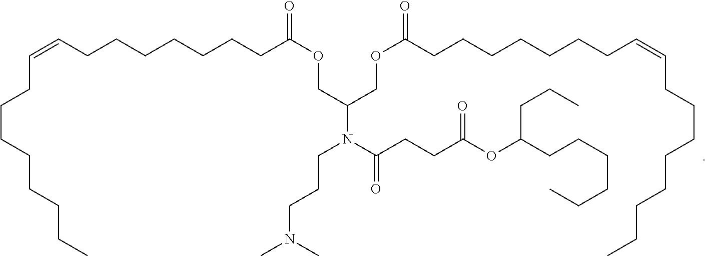 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

EMBODIMENT 200. The targeted lipid nanoparticle of any one of EMBODIMENTS 170-199, whereby the inserting occurs at a rotational speed of zero revolutions per minute (0 RMPs) during the time-period.

EMBODIMENT 201. The targeted lipid nanoparticle of any one of EMBODIMENTS 170-199, whereby the inserting occurs at a rotational speed of no more than three hundred revolutions per minute (300 RMPs) during the time-period.

EMBODIMENT 202. The targeted lipid nanoparticle of any one of EMBODIMENTS 170-199, whereby the inserting occurs at a rotational speed of no more than six hundred revolutions per minute (600 RMPs) during the time-period.

EMBODIMENT 203. The targeted lipid nanoparticle of any one of EMBODIMENTS 170-202, whereby the nucleic acid cargo within the targeted lipid nanoparticles is less than 10% degraded compared to a nucleic acid cargo in a particle where the targeting moiety is not inserted by the process.

EMBODIMENT 204. The targeted lipid nanoparticle of any one of EMBODIMENTS 170-203, wherein the lipid nanoparticle ranges from approximately 50 nanometers to 200 nanometers in size.

EMBODIMENT 205. The targeted lipid nanoparticle of any one of EMBODIMENTS 170-203, wherein the lipid nanoparticle ranges from approximately 75 nanometers to 85 nanometers in size.

EMBODIMENT 206. The targeted lipid nanoparticle of any one of EMBODIMENTS 170-203, wherein the lipid nanoparticle ranges from approximately 85 nanometers to 97 nanometers in size.

EMBODIMENT 207. The targeted lipid nanoparticle of any one of EMBODIMENTS 170-203, wherein the lipid nanoparticle ranges from approximately 80 nanometers to 90 nanometers in size.

EMBODIMENT 208. The targeted lipid nanoparticle of any one of EMBODIMENTS 170-207, whereby the insertion of the targeting moiety into the lipid nanoparticle increases the size of the lipid nanoparticle between 5% to 25%.

EMBODIMENT 209. The targeted lipid nanoparticle of any one of EMBODIMENTS 170-208, whereby the insertion of the targeting moiety into the lipid nanoparticle increases the size of the lipid nanoparticle by about 20%.

EMBODIMENT 210. The targeted lipid nanoparticle of any one of EMBODIMENTS 170-209, whereby the targeted nanoparticle is stable for a time-period of at least 5 days at a temperature between 2° C. and 8° C.

EMBODIMENT 211. The targeted lipid nanoparticle of any one of EMBODIMENTS 170-210, whereby the nucleic acid cargo within the targeted lipid nanoparticle remains substantially intact for at least 3 freeze thaw cycles.

EMBODIMENT 212. The targeted lipid nanoparticle of any one of EMBODIMENTS 170-211, wherein the lipid nanoparticle comprises an amount of an ionizable lipid, an amount of cholesterol, an amount of neutral lipid, and an amount of a PEG-lipid.

EMBODIMENT 213. The targeted lipid nanoparticle of EMBODIMENTS 212, wherein the lipid nanoparticle comprises amounts of the ionizable lipid, the cholesterol, the neutral lipid, and the PEG-lipid at molar ratio ranges respectively of 40%-60% ionizable lipid, 20%-35% cholesterol, 10%-30% of neutral lipid, 0.5%-5% of PEG-lipid.

EMBODIMENT 214. The targeted lipid nanoparticle of EMBODIMENTS 212, wherein the ionizable lipid is selected from the group consisting of a Lipid 1, a Lipid 2, a Lipid 3, a Lipid 4, a Lipid 5, a Lipid 6, a Lipid 7, a Lipid 8, a Lipid 9, and a Lipid 10 ionizable lipid.

EMBODIMENT 215. The targeted lipid nanoparticle of EMBODIMENTS 212, wherein the ionizable lipid is selected from the group consisting of a KC2 lipid, a DLin-MC2-DMA lipid, a DLin-MC3-DMA lipid, a DSDMA lipid, a DODMA lipid, a DLinDMA lipid, a DLenDMA lipid, a γ-DLenDMA lipid, a DLin-K-DMA lipid, a DLin-C2K-DMA lipid, a DLin-K-C3-DMA lipid, a DLin-K-C4-DMA lipid, a DLen-C2K-DMA lipid, a γ-DLen-C2K-DMA lipid, or a DLin-MP-DMA lipid.

EMBODIMENT 216. The targeted lipid nanoparticle of EMBODIMENTS 212, wherein the neutral lipid is a phosphatidylcholine lipid or a phosphatidylethanolamine lipid.

EMBODIMENT 217. The targeted lipid nanoparticle of EMBODIMENTS 212, wherein the phosphatidylcholine lipid or the phosphatidylethanolamine lipid is selected from the group consisting of DOPE, DSPC, DPPC, and POPC.

### EXAMPLES

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

### Example 1: Process Development for Insertion of a Targeting Moiety into a Lipid Nanoparticle

The following describes process development parameters evaluating quantity requirements of a Fab targeting moiety for insertion into a lipid nanoparticle. The example also assessed the impact of reaction time at a fixed temperature of 37° C. In this example, lipid nanoparticles encapsulating nucleic acid cargos encoding reporter molecules (luciferase mRNA or GFP-mRNA) were combined with different amounts of Fab, namely 0.15 μg-30 g Fab per mol of total lipid in the reaction, for a time-period ranging from 4-14 hours). Targeted lipid nanoparticles were produced by the reaction and their size, zeta potential (mV), encapsulation efficiency (%) was assessed.

### Materials and Methods

### Insertion Process

Insertion of the targeting ligand micelle into a pre-formed LNP is performed either using Thermomixer C (Eppendorf) or a BioFlo® reactor (Eppendorf). A pre-determined ratio of the targeting ligand micelle and LNP are mixed together in the reaction container and incubated at a pre-determined temperature and mixing speed for pre-determined amount of time. At the end of the incubation period the reaction is quenched using a heat exchanger to form targeted lipid nanoparticles.

### LNP Formation

The cargo to be encapsulated is diluted in a low pH buffer and mixed with the desired lipid mixture in ethanol at a predetermined ratio and total flow rate. The resultant solution with ethanol is then buffer exchanged into a specific formulation buffer to obtain the pre-formed LNPs.

### Size and Zeta Potential Measurements

LNP size and zeta potential were measured using a Malvern Nano Zetasizer (Malvern Panalytical). Prior to measurement, LNPs were diluted in PBS or a specific formulation matrix used for manufacturing.

mRNA encapsulation mRNA encapsulation was measured using the Quant-iT™ RiboGreen™ RNA Reagent and Kit (Invitrogen). A standard curve was prepared by diluting the mRNA solution in 1×PBS to achieve a serial dilution of mRNA at different concentrations. The non encapsulated mRNA concentration was measured by adding the LNPs to the fluorescent dye. The total mRNA concentration was measured by lysing the LNPs using Triton X-100 and then mixing with the fluorescent dye. Encapsulated mRNA content was then calculated as a difference between the total mRNA and non-encapsulated mRNA.

### In-Vitro Test

hCMEC/D3 cells ((Millipore, SCC066), BEND3 (ATCC, CRL-2299) cells or human T-cells were plated on a 96-well tissue culture plates. 24 hours post plating, targeted LNPs encapsulating mRNA cargo were added to the cells at different dosage. 24 hours post treatment, cells were rinsed, resuspended and the expression per cell was analyzed using Fluroscence Activated Cell Sorting flow cytometry technique (NovoCyte Penteon).

mRNA Integrity

mRNA was released and extracted through the deformulation process of LNP. The extracted mRNA was prepared both heat with specific temperature and time and no heat and followed by mixing with the sample buffer provided by Revvity. The prepared samples were then injected into a microchip loaded with proprietary gels that can create the separation based on mRNA size. All species of mRNA were determined through the labeling of a fluorescent dye.

### Lipid Content

LNP samples were diluted with methanol with a certain dilution factor and then injected in a phenyl-hexyl column on a UPLC to achieve the separation of each lipid. The content of the lipid was measured with a Charge Aerosol Detector (CAD) and a standard curve generated with the lipid with the known concentration.

TABLE 1

| | | Zeta | Encapsulation |
|---|---|---|---|
| Targeted lipid | Size | Potential | Efficiency |
| nanoparticles | (nm) | (mV) | (%) |
| LNP (GFP-mRNA) | 75-85 | 2-10 | 80-90 |
| LNP (luciferase-mRNA) | 80-90 | 2-10 | 80-90 |
| LNP (GFP-mRNA)-Fab1 | 82-96 | N/A | N/A |
| LNP (GFP-mRNA)-Fab2 | 85-97 | | |
| LNP (GFP-mRNA)-Fab3 | 80-90 | | |
| LNP (GFP-mRNA)-Fab4 | 80-90 | | |
| LNP (GFP-mRNA)-Fab5 | 80-90 | | |
| LNP (GFP-mRNA)-Fab6 | 80-90 | | |
| LNP (luciferase-mRNA)- | 84-96 | | |
| Fab1 | | | |
| LNP (luciferase-mRNA)- | 80-95 | | |
| Fab3 | | | |
| LNP (luciferase-mRNA)- | 80-95 | | |
| Fab4 | | | |
| LNP (luciferase-mRNA)- | 80-95 | | |
| Fab5 | | | |
| LNP (luciferase-mRNA)- | 80-95 | | |
| Fab6 | | | |

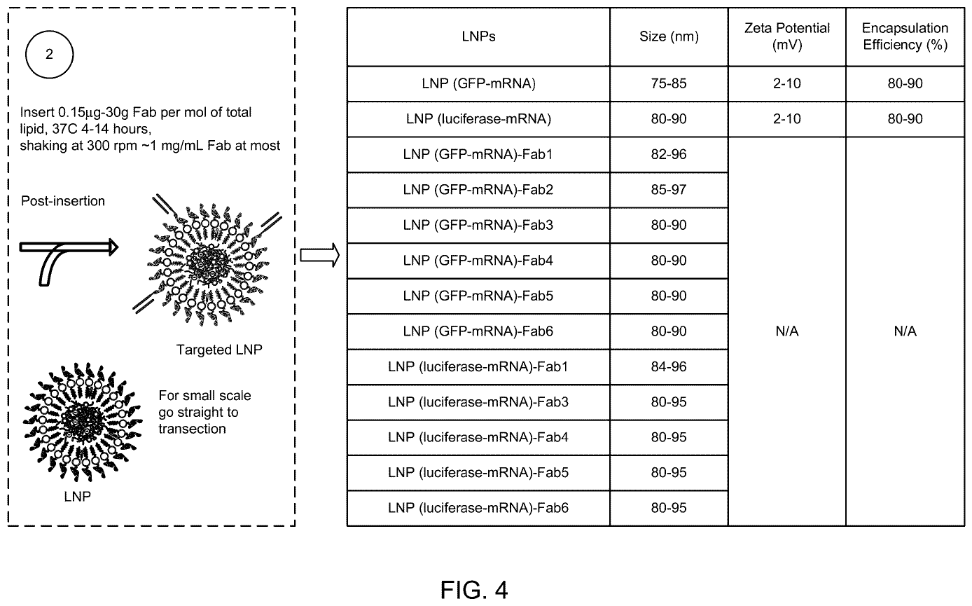

FIG. 4 is a chart outlining various physical parameters, including size, zeta potential (mV), and encapsulation efficiency (%) of targeted lipid nanoparticles produced by a process of the disclosure. The described particles were produced by insertion of a fragment antigen-binding (Fab) that specifically binds a human transferrin targeting moiety into lipid nanoparticle(s) (LNP) encapsulating reporter nucleic acid cargos (GFP-mRNAs or luciferase-mRNAs) by mixing the two at 37° C. for 4 hours.

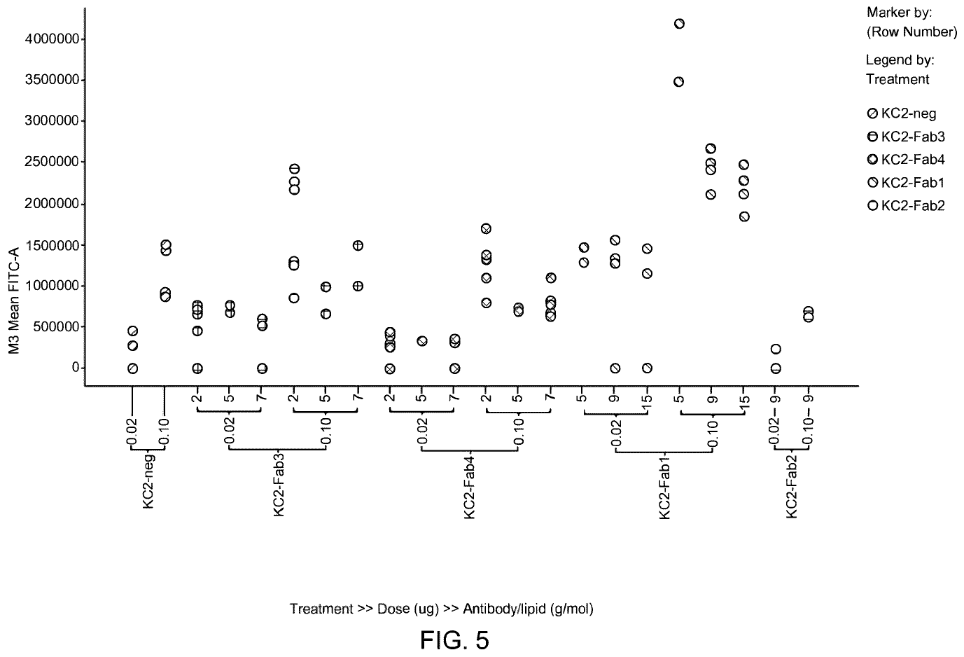

FIG. 5 is a chart outlining mean FITC-A expression in Bend3 murine cell line of the reporter nucleic acid cargos (GFP-mRNAs) as a function of mRNA dose into the cells. Overall, at least in part because the targeting moiety was specific to human TfR, the murine cells proved difficult to transfect. FIG. 5 shows an increase in GFP expression with an increase in mRNA dose.

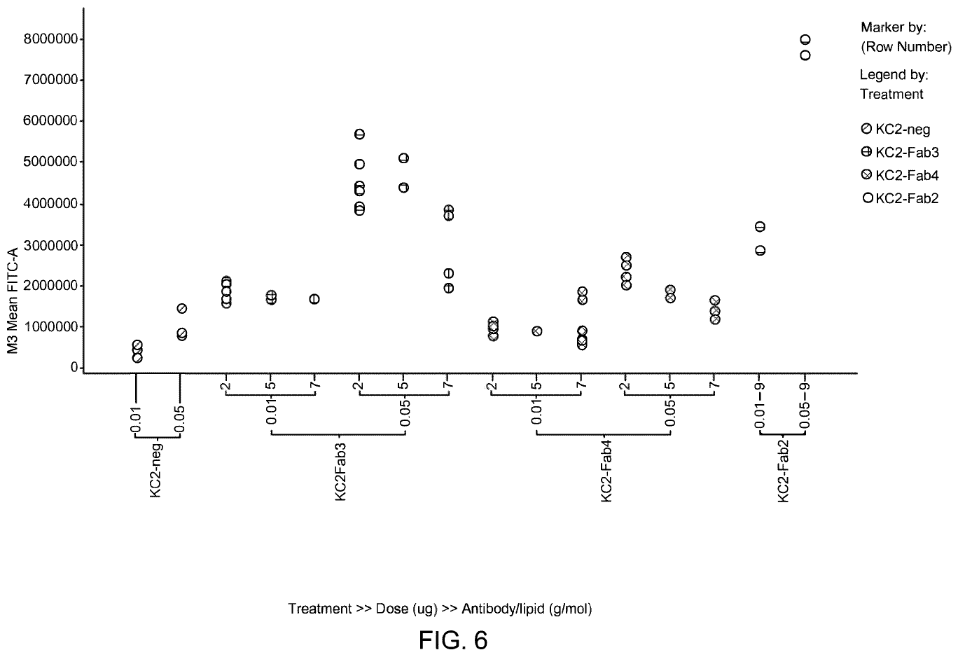

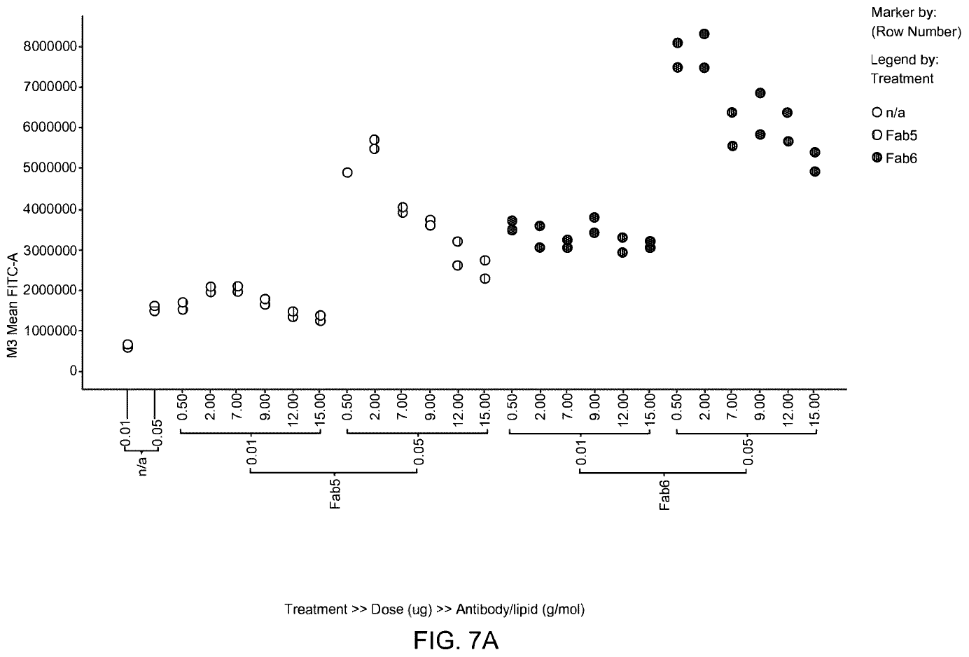

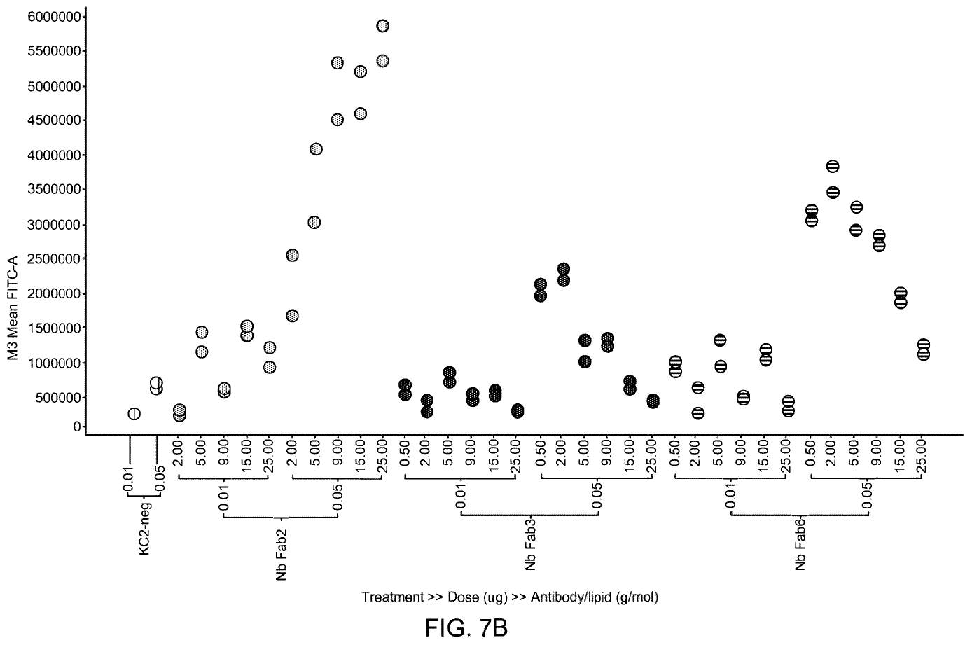

FIG. 6 is a chart outlining mean FITC-A expression in Bend3 murine cell line of the reporter nucleic acid cargos (GFP-mRNAs) as a function of mRNA dose into the cells and antibody grafting density (i.e., Ratio of antigen binding moiety/Fab to mRNA or antigen binding moiety-Fab to lipids). FIG. 6 shows an increase in GFP expression with an increase in mRNA dose. FIGS. 7A-7B (FIG. 7A-7B) are charts outlining mean FITC-A expression in hCMEC cell lines as a function of mRNA dose. FIGS. 7A-7B provide an expanded grafting density impact assessment.

### Example 2: Process Development for Insertion of a Targeting Moiety into a Lipid Nanoparticle

The following describes process development parameters and stability summary observed during the manufacturing of lipid nanoparticles.

### Materials and Methods

### Insertion Process

Insertion of the targeting ligand micelle into a pre-formed LNP is performed either using Thermomixer C (Eppendorf) or a BioFlo® reactor (Eppendorf). A pre-determined ratio of the targeting ligand micelle and LNP are mixed together in the reaction container and incubated at a pre-determined temperature and mixing speed for pre-determined amount of time. At the end of the incubation period the reaction is quenched using a heat exchanger to form targeted lipid nanoparticles.

### LNP Formation

The cargo to be encapsulated is diluted in a low pH buffer and mixed with the desired lipid mixture in ethanol at a predetermined ratio and total flow rate. The resultant solution with ethanol is then buffer exchanged into a specific formulation buffer to obtain the pre-formed LNPs.

TABLE 2

| | Data Summary | | |
|---|---|---|---|
| | LNP Size | Encapsulation | |
| | (Ph 1 | Efficiency | |
| | proposed | (Ph 1 proposed | |
| Targeting Moiety | spec: | spec: ≥80%) | In-vitro |
| insertion buffer | 50-200 nm) | nm) | Expression |
| 25 mM MES, | No impact | No impact after | Nominal change |
| 150 mM NaCl, | after 5 days at | 5 days at 25 C. | at dose of 1.0 |
| 10% sucrose, | 25 C. and 2-8 C. | and 2-8 C. | and 0.3 μg/mL |
| pH 6.5 | Nominal | Nominal | |
| | increase in | change post 3x | |
| | LNP size post | F/T | |
| | 3x F/T (100 to | (freeze/thaw) | |
| | 110 nm) | | |
| 25 mM MES, | No impact | No impact after | Nominal change |
| 150 mM NaCl, | after 5 days at | 5 days at 25 C. | at dose of 1.0 |
| 10% sucrose, | 25 C. and 2-8 C. | and 2-8 C. | and 0.3 μg/mL |
| pH 6.5 | Nominal | Nominal | |
| | increase in | change post | |
| | LNP size post | 3xF/T | |
| | 3x F/T (90 to | | |
| | 110 nm) | | |
| 25 mM HEPES, | No impact | No impact after | Nominal change |
| 150 mM NaCl, | after 5 days at | 5 days at 25 C. | at 1.0 μg/mL |
| 10% sucrose, | 25 C. and 2-8 C. | and 2-8 C. | 30-50% drop |
| pH 7.4 | Increase in | Nominal | in expression |
| | LNP size post | change post | at 0.3 μg/mL |
| | 3x F/T (70 to | 3xF/T | |
| | 110 nm) | | |
| 25 mM HEPES, | No impact | No impact after | 30-50% drop in |
| 150 mM NaCl, | after 5 days at | 5 days at | expression at |
| 10% sucrose, | 25 C. and 2-8 C. | 25 C. and | both 0.1 & |
| pH 7.4 | Increase in | 2-8 C. ~15% | 0.3 μg/mL |
| | LNP size post | decrease post | |
| | 3x F/T (70 to | 3xF/T | |
| | 110 nm) | | |

### Results

The following study describes the impact of temperature, incubation time, and mixing parameters into the incorporation of a targeting moiety into a lipid nanoparticle. In this example a variety of targeted lipid nanoparticles carrying nucleic acid cargos encoding reporter molecules were prepared under different conditions (e.g., insertion for a time-period of 4-14 hours) and assessed for the insertion process effect in the size, zeta potential (mV), encapsulation efficiency (%) and targeting efficiency of the resulting LNPs.

### Materials and Methods

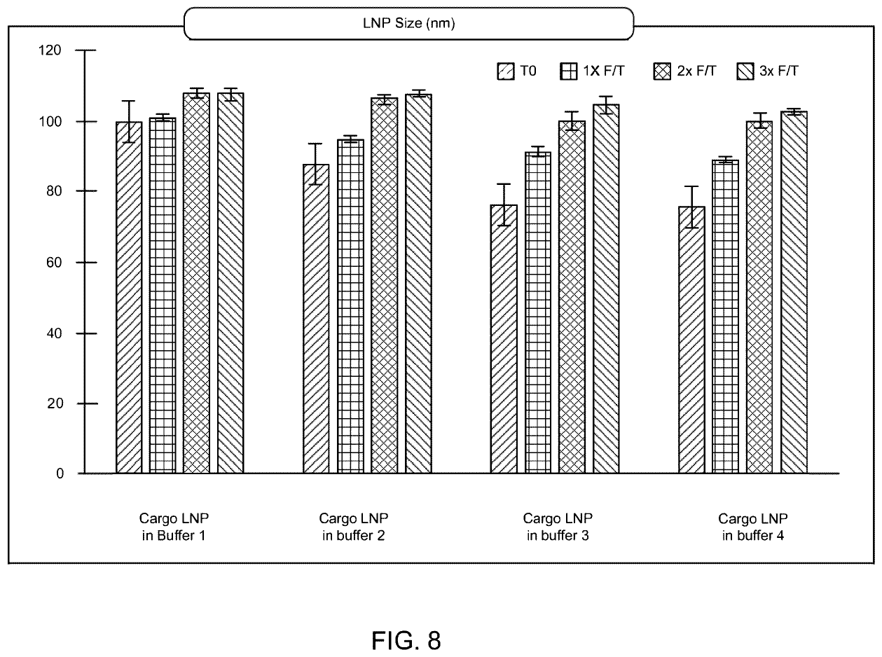

FIG. 8 (FIG. 8) is a chart outlining the total targeted lipid nanoparticle size (nm) of targeted lipid nanoparticles encapsulating a nucleic acid encoding a cargo in various buffers. The Y-axis depicts the impact in the size of the targeted lipid nanoparticle (nm) as a function of repeated freeze thaw (F/T) cycles.

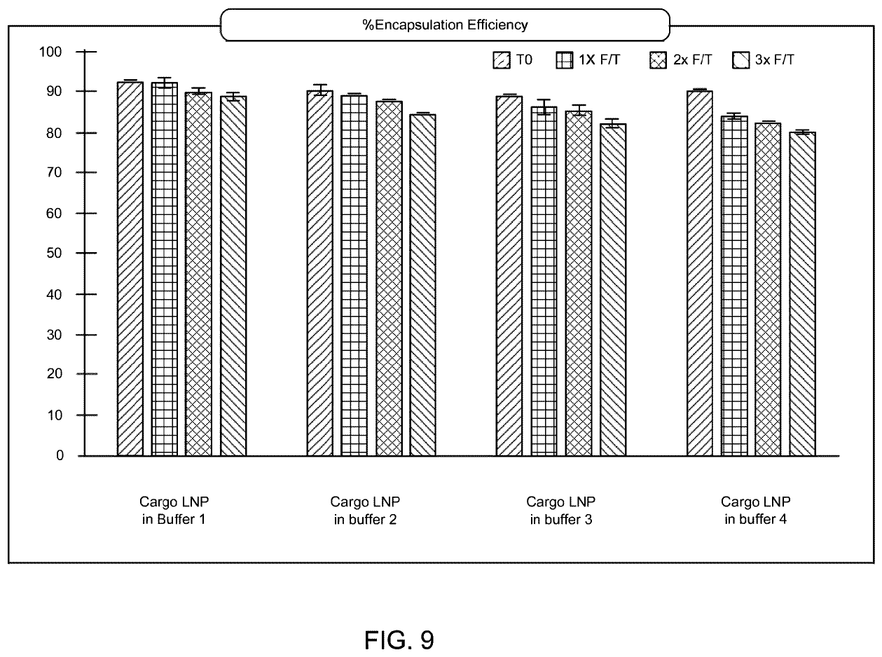

FIG. 9 (FIG. 9) is a chart outlining the percent (%) encapsulation efficiency of targeted lipid nanoparticles encapsulating a nucleic acid encoding a cargo in various buffers. The Y-axis depicts the impact in the size of the targeted lipid nanoparticle (nm) as a function of repeated freeze thaw (F/T) cycles.

### Example 3: Process Development for Insertion of a Targeting Moiety into a Lipid Nanoparticle

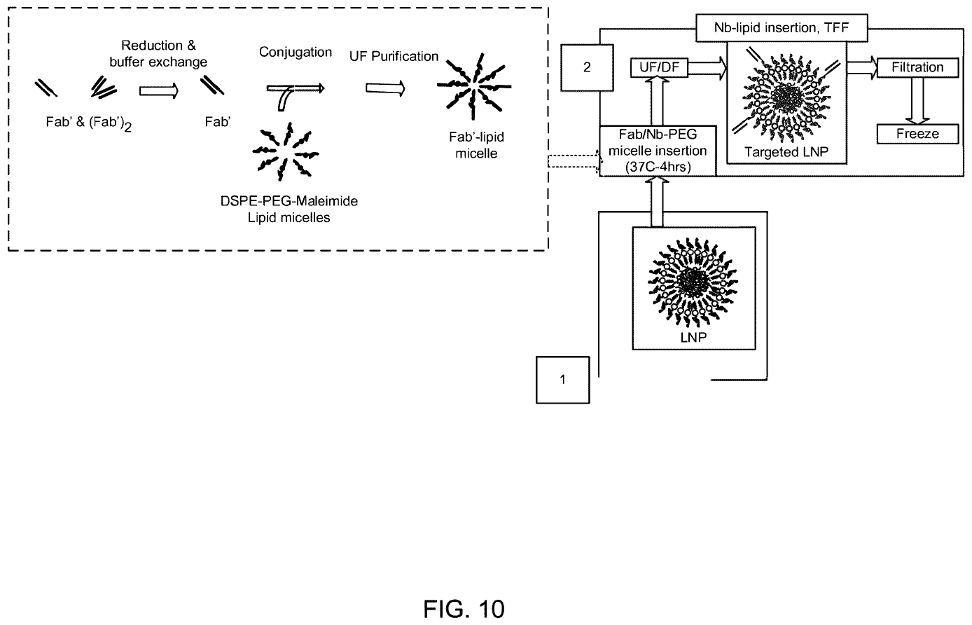

The following describes process development parameters evaluating quantity requirements of a Fab targeting moiety for insertion into a lipid nanoparticle. In this example, a Fab targeting moiety is incorporated into a lipid nanoparticle via DSPE-PEG-Maleimide linker lipid micelles (see FIG. 10). The example also assessed the impact of reaction time of 4 hours at 37° C., with quenching the reaction at room temperature. The impact of various buffers on the stability of the targeted lipid nanoparticle, the integrity of the nucleic acid cargo, and stability of the targeted lipid nanoparticle to freeze thaw cycles is evaluated.

### Methods

In this example, Fab′-lipid micelles are incorporated into lipid nanoparticles encapsulating nucleic acid cargos encoding a test nucleic acid cargo, namely 0.5 μg-25 g Fab per mol of total lipid in the reaction. Targeted lipid nanoparticles were produced by the reaction and their size, zeta potential (mV), encapsulation efficiency (%) was assessed. Materials and Methods used are substantially as described in Example 1.

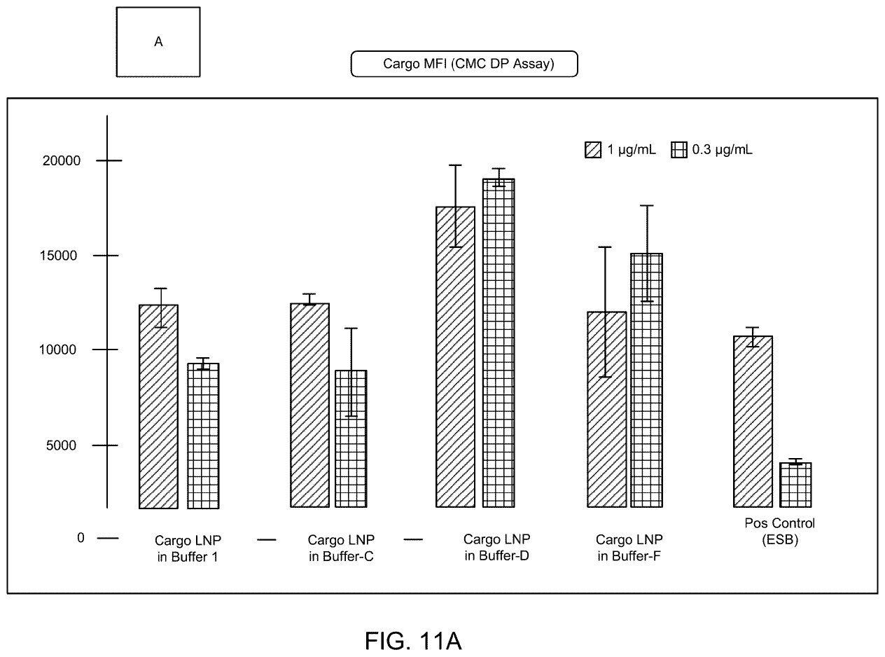

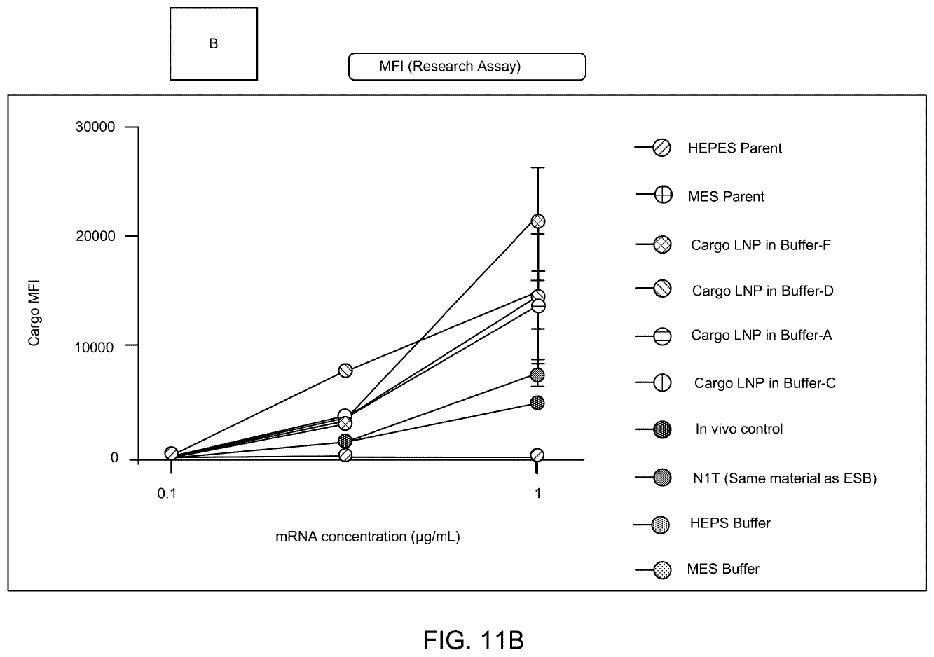

FIG. 11A-B (FIG. 11A-B) are charts evaluating the impact of various buffers on the stability of the nucleic acid cargo and the targeted lipid nanoparticle itself as measured by the mean fluorescence intensity of the nucleic acid cargo.

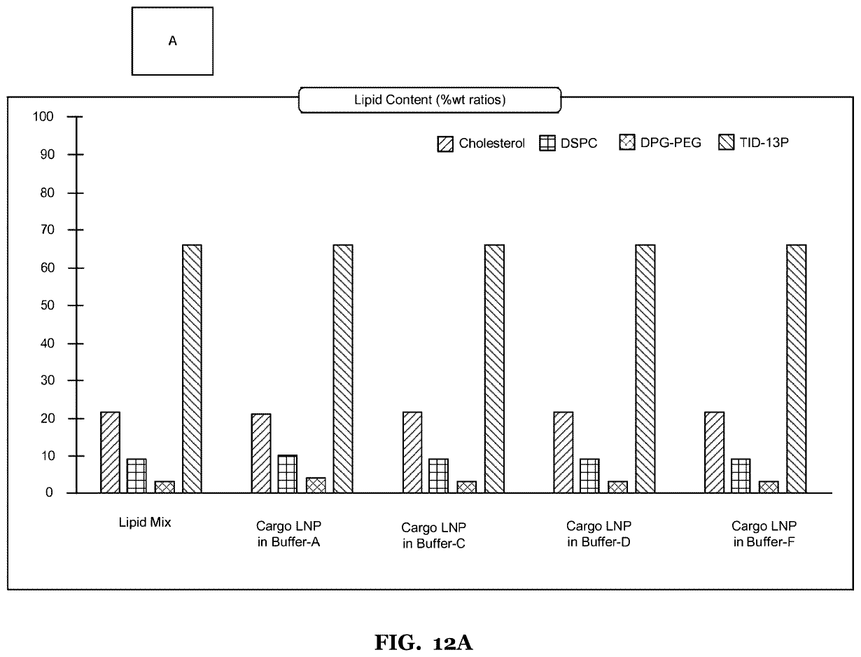

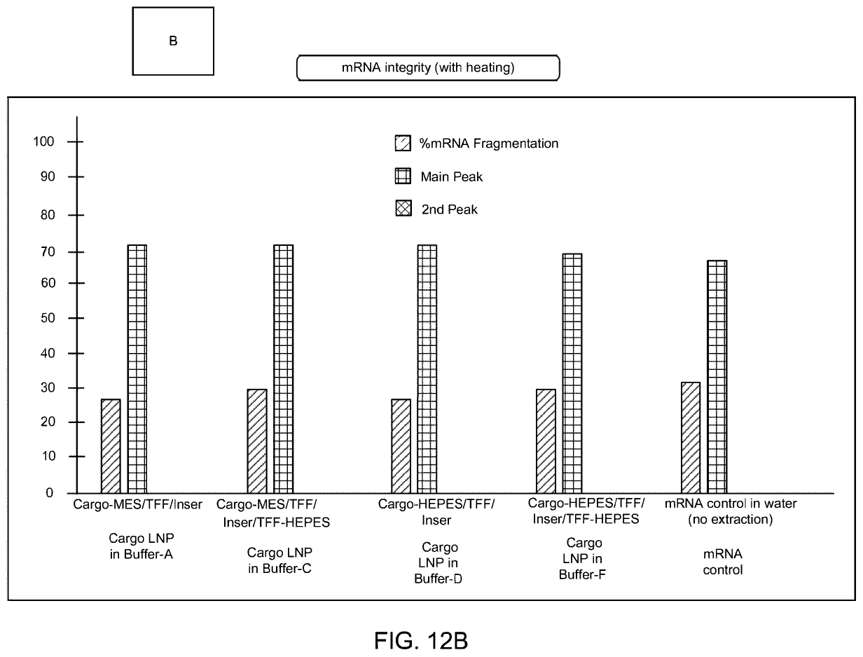

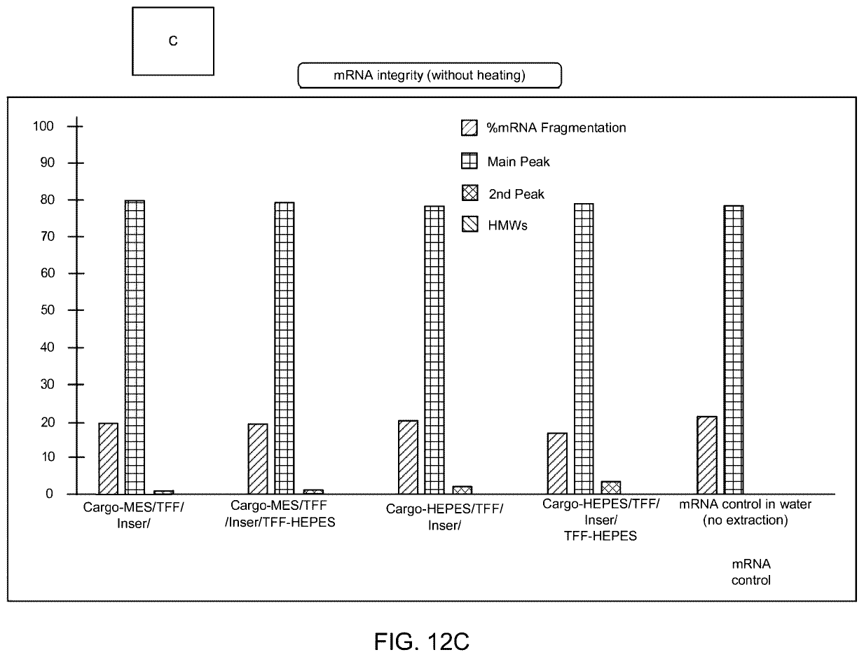

FIG. 12A-C (FIG. 12A-C) are charts outlining various biophysical properties of lipid nanoparticles manufactured in distinct buffers. FIG. 12A is a chart outlining the percent (%) distribution of various lipids in a targeted lipid nanoparticle. FIGS. 12A-B are charts outlining the integrity of the mRNA cargos after insertion of a targeting moiety when heating is used FIG. 12B and when heating is not FIG. 12C used in the reaction.

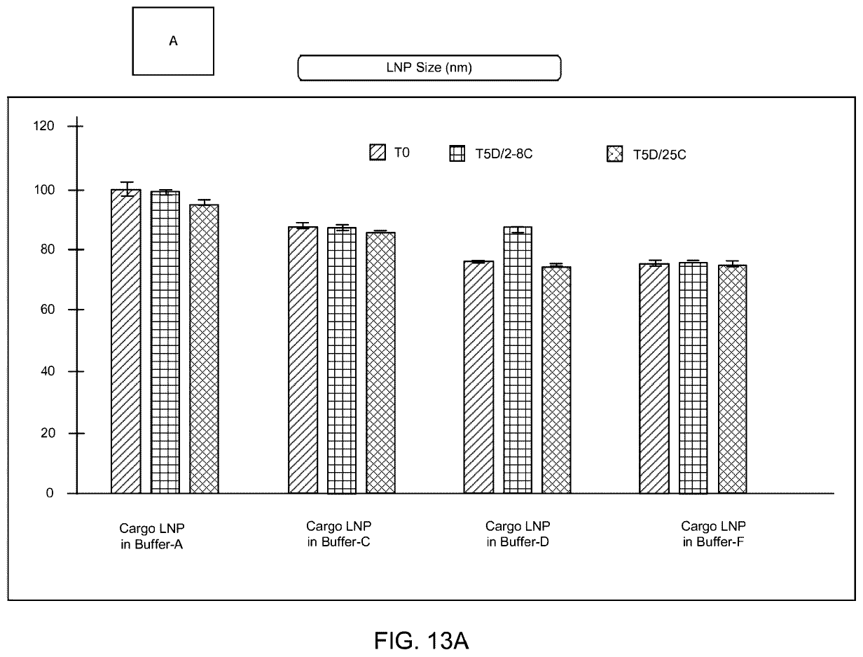

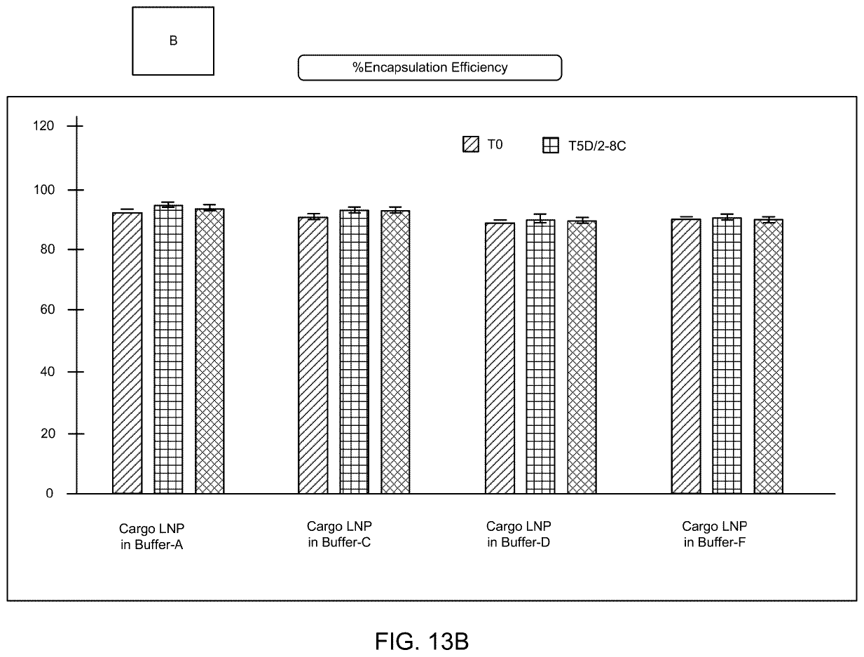

FIG. 13A-B (FIG. 13A-B) are charts outlining various biophysical properties of lipid nanoparticles manufactured as described herein. FIG. 13A is a chart outlining the thermostability of the targeted lipid nanoparticle encapsulating a nucleic acid encoding a cargo in various buffers. As shown in FIG. 13A T0=time zero, T5D/2-8° C. (thermostability for 5 days between 2-8° C.), and T5D/25 (thermostability for 5 days at 25° C.). FIG. 13B is a chart outlining the relationship between the thermostability of the lipid nanoparticles and the percent encapsulation efficiency of the targeted lipid encapsulating a nucleic acid encoding a cargo in various buffers.

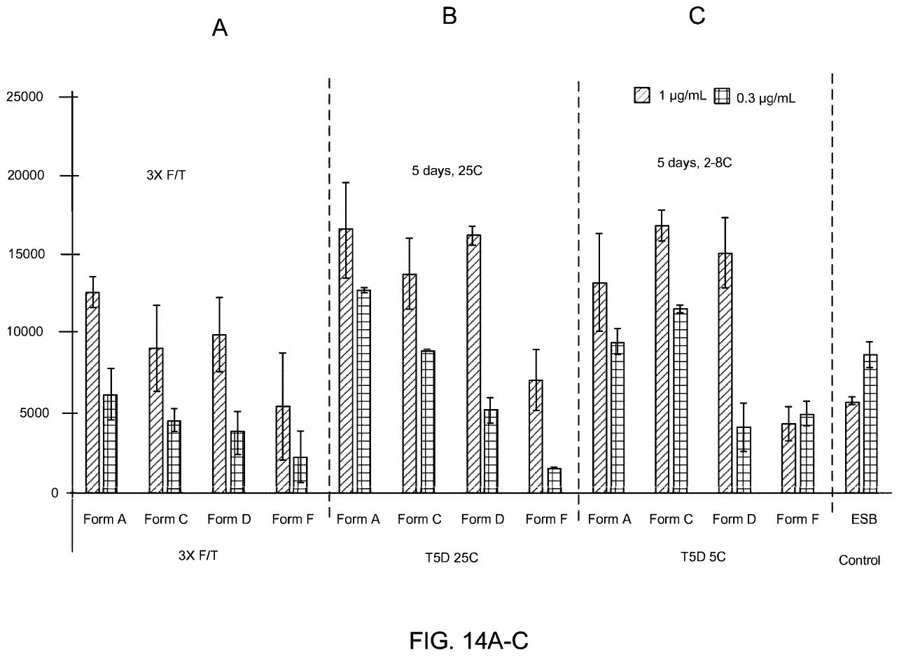

FIG. 14A-C (FIG. 14A-C) are charts evaluating the impact of various properties of lipid nanoparticles manufactured as described herein. FIG. 14A is a chart depicting the impact of various freeze thaw (F/T) cycles. FIG. 14B depicts the thermostability of the nanoparticle at T5D/25 (thermostability for 5 days at 25° C.). FIG. 14C depicts the thermostability of the nanoparticle for 5 days between 2-8° C.).

### Example 4: Impact of Temperature, Incubation Time, and Mixing

The following describes process development parameters evaluating the impact of temperature, incubation time, and mixing.

### Methods

Antigen binding protein-PEG micelle was inserted into a pre-formed LNP using a Thermomixer C. A pre-determined ratio of 168.7 mg of the targeting ligand micelle per g of encapsulated nucleic acid are mixed together in the reaction container and incubated at different temperatures (20° C., 37° C., 54° C.), mixing speed of (0, 300, 600 RPM) for a period of 0.5, 4 and 8 hours. Decrease in encapsulation efficiency was observed after incubation at 54° C. Hence the temperature range for further evaluation was narrowed down to 20° C. to 42° C. No impact was seen of the mixing rate. Other materials and methods used are substantially as described in Example 1.

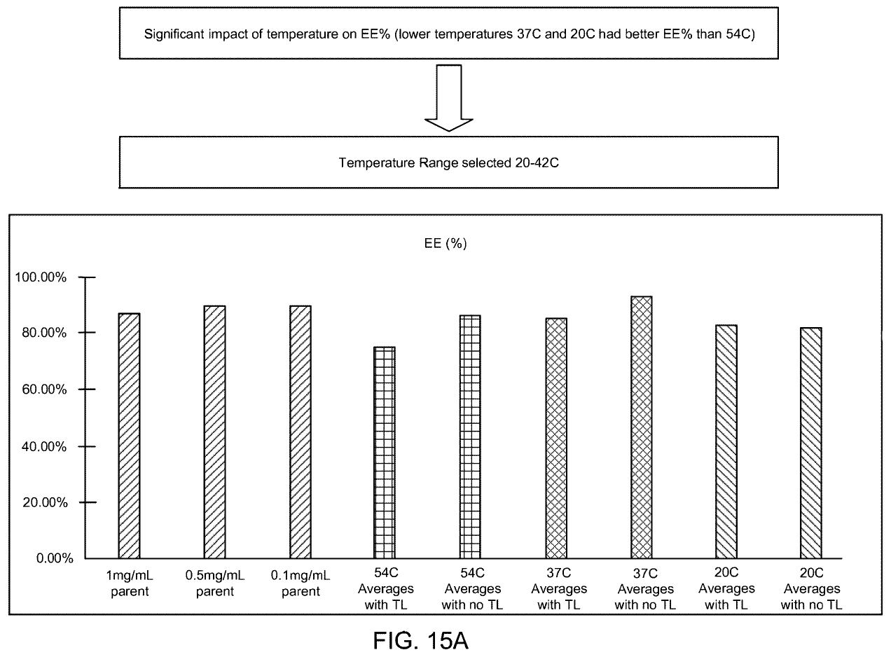

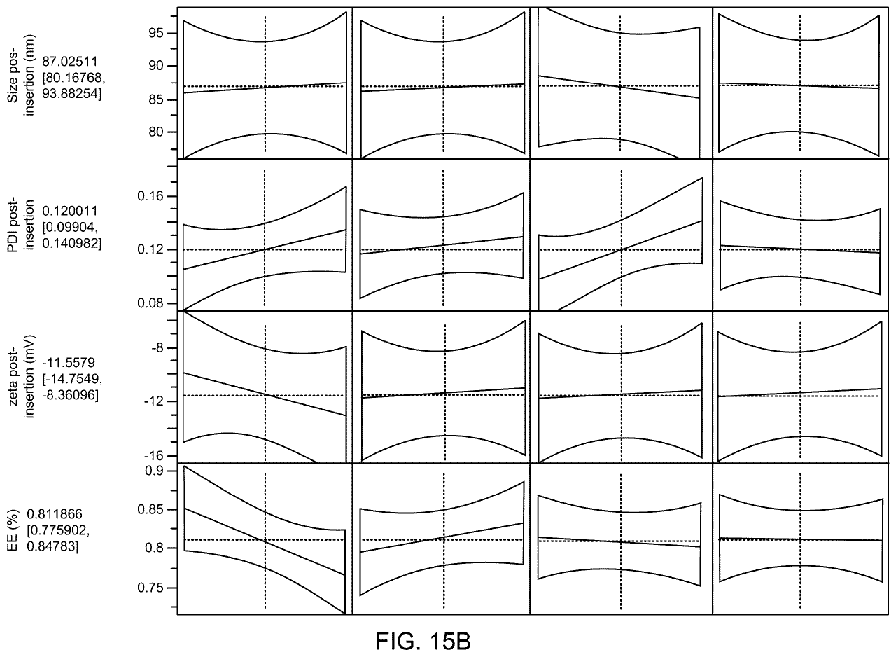

FIGS. 15A-B (FIG. 15A-B) are charts evaluating the impact of temperature, incubation time, and mixing in processes for insertion of targeting moieties into lipid nanoparticles.

### Example 5: Impact of Preheating

The following describes process development parameters evaluating the impact of temperature, incubation time, and mixing. The materials and methods are substantially as described in prior examples.

### Methods

Antigen binding protein-PEG micelle was inserted into a pre-formed LNP using a Thermomixer C. The incubation conditions were kept constant (temperature: 37° C., time: 4 hours, mixing speed: 300 RPM). Before mixing the antigen binding protein-PEG micelle with LNPs either the individual components or both were preheated to 37° C. or only heated after being mixed together. Increase in Size and lower in-vitro expression was observed when antigen binding protein-micelles are pre-heated. No impact was observed for all other conditions.

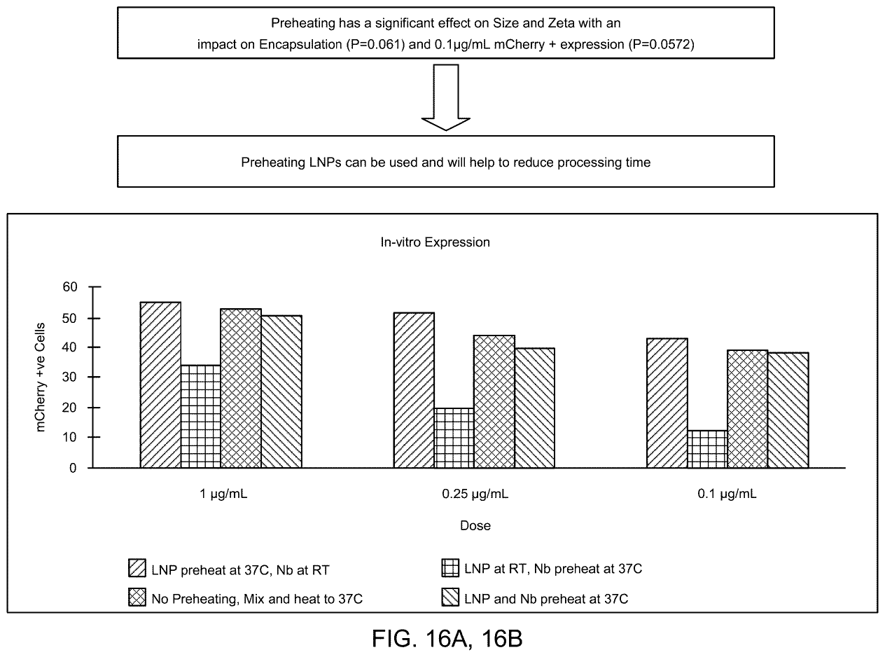

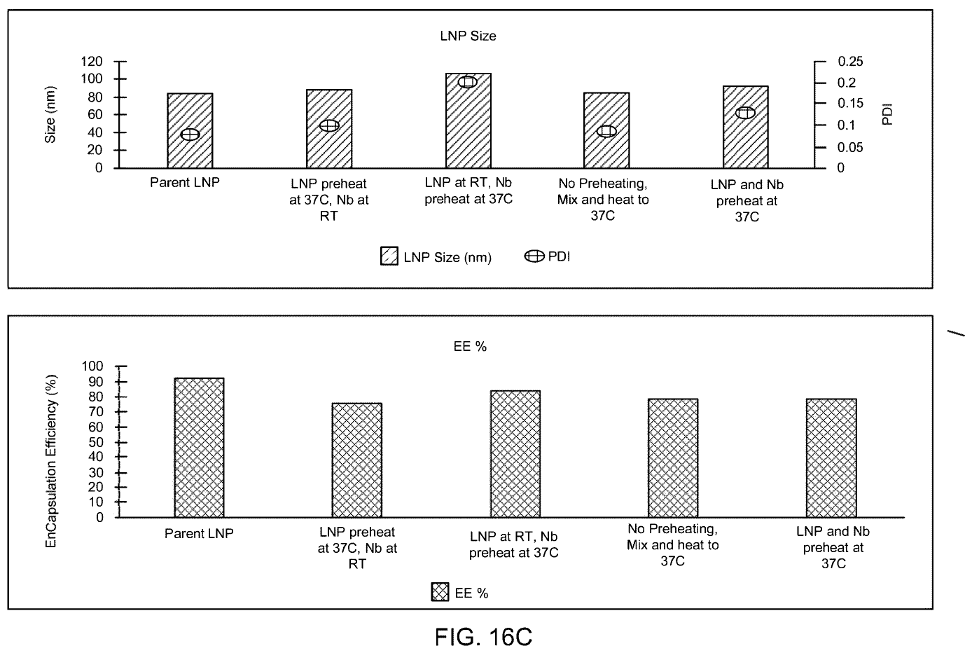

FIGS. 16A-C (FIG. 16A-C) are charts evaluating the impact of pre-heating in processes for insertion of targeting moieties into lipid nanoparticles.

### Example 6: Impact of Preheating

The following describes process development parameters evaluating the impact of incubation time and temperature for a targeted nucleic acid lipid nanoparticle encompassing an mRNA encoding a VHH cargo. The materials and methods are substantially as described in prior examples.

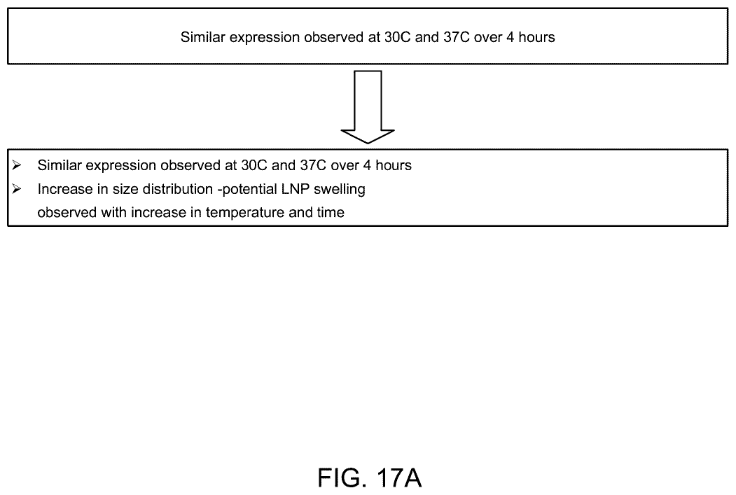

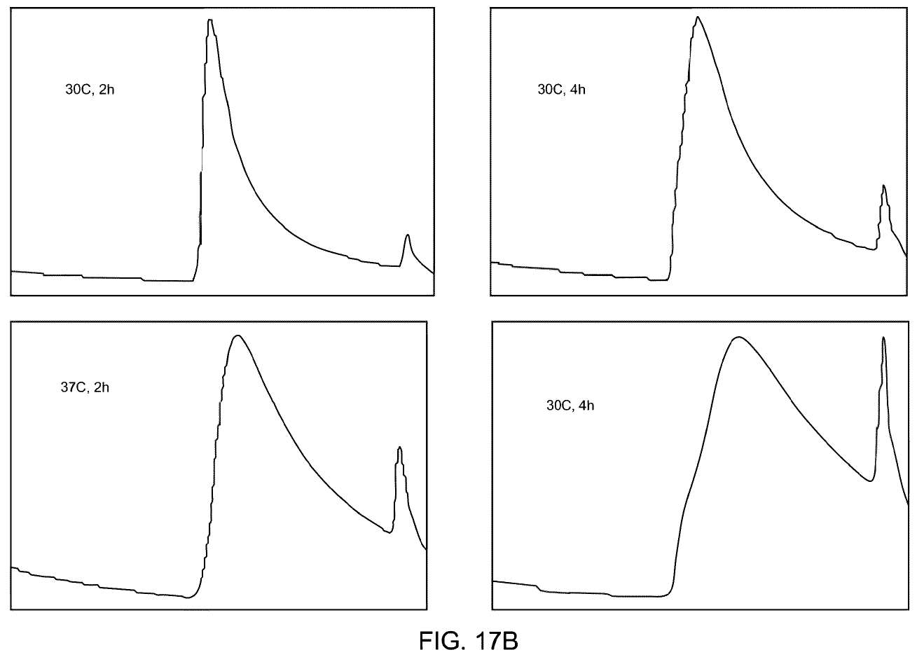

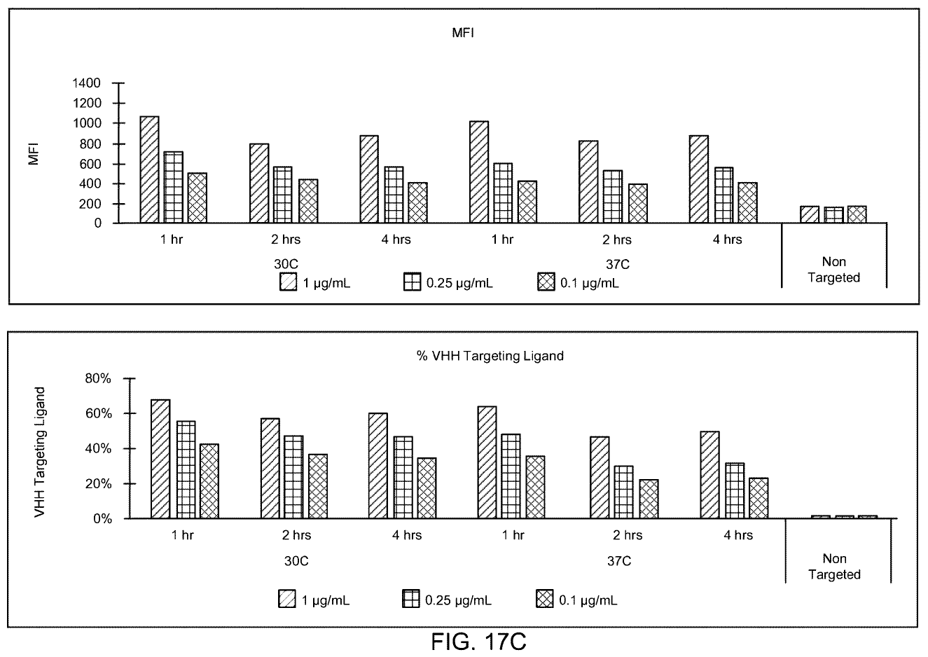

FIGS. 17A-C (FIG. 17A-C) are charts evaluating the impact of targeting moiety incubation time and temperature in processes for insertion of targeting moieties into lipid nanoparticles.

### Methods

Antigen binding protein-PEG micelle was inserted into a pre-formed LNP using a Thermomixer C. A pre-determined ratio of 168.7 mg of the targeting ligand micelle per g of encapsulated nucleic acid are mixed together in the reaction container and incubated at different temperatures (20° C., 30° C., 37° C., 42° C.), at mixing speed of 300 RPM for a period of 0.5, 1, 2 and 4 hours. No impact on encapsulation efficiency was observed. Similar in-vitro expression was observed at 30 C and 37 C. Increase in size distribution-potential LNP swelling observed with increase in temperature at 37° C. and time to 4 hours.

### Example 7: Insertion of an HSP34 Fab-PEG Micelle Targeting Moiety into LNPs with Distinct Lipid Compositions

The following describes process development parameters evaluating the impact of incubation time and temperature for a targeted nucleic acid lipid nanoparticle encompassing an mRNA reporter cargo. The materials and methods are substantially as described in prior examples.

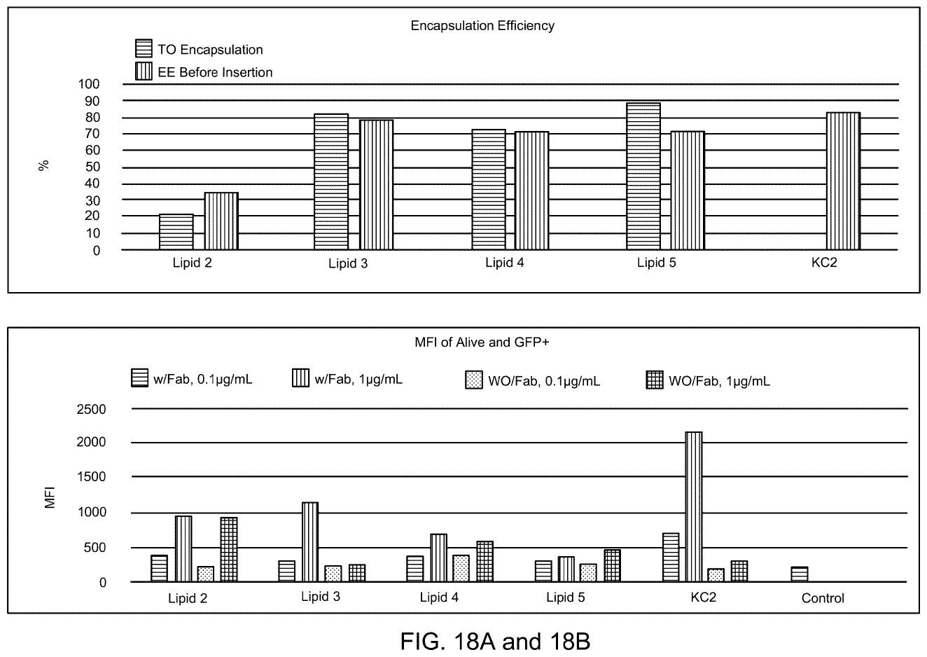

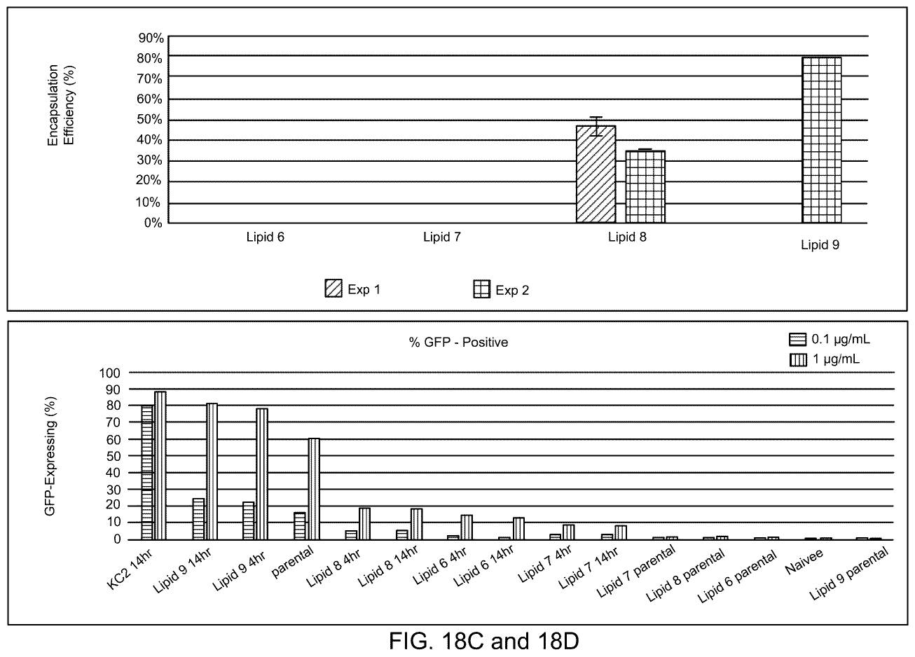

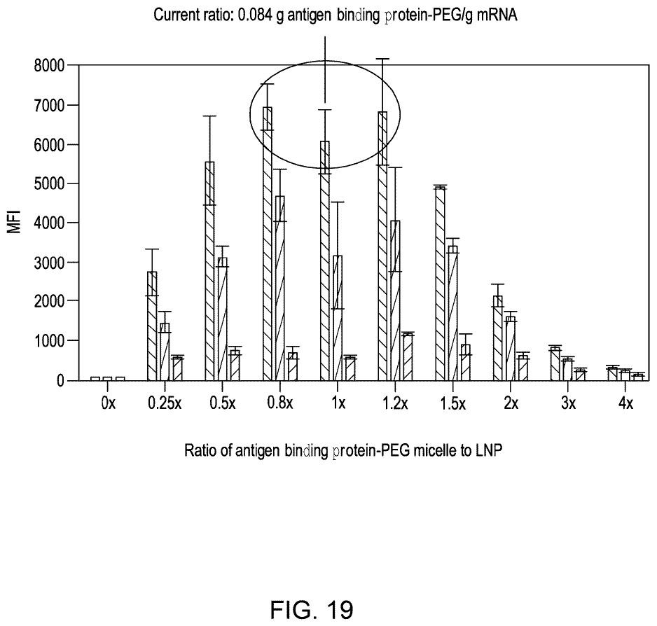

FIGS. 18A-D (FIG. 18A-D) are charts evaluating processes for insertion of targeting moieties into lipid nanoparticles with different lipid types. FIG. 19 (FIG. 19) is a chart evaluating the effects of antigen binding protein to LNP ratio.

### Example 8: Fab-PEG Micelle Insertion and Effects on LNP Properties

Additional studies were conducted to evaluate the impact of Fab-PEG micelle insertion and downstream processing steps on the physical properties and stability of LNPs within a manufacturing process. LNPs were prepared as described above, and following initial formulation, were subjected to buffer exchange (Amicon), sucrose spike-in, and filtration. The LNPs were then stored at 2-8° C. prior to Fab-PEG micelle insertion, which was evaluated at different insertion conditions outlined on the table below.

TABLE 3

| | Insertion | | | |
|---|---|---|---|---|
| | temperature | Insertion time | | |
| LNP# | (C.) | (hour) | Speed (rpm) | Pre-heating |
| LNP #1 | 20 | 0.5 | 0 | No |
| LNP #2 | 20 | 3.5 | 600 | No |
| LNP #3 | 20 | 0.5 | 0 | No |
| LNP #4 | 20 | 3.5 | 600 | No |
| LNP #5 | 40 | 0.5 | 0 | No |
| LNP #6 | 40 | 3.5 | 600 | No |
| LNP #7 | 40 | 0.5 | 0 | No |
| LNP #8 | 40 | 3.5 | 600 | No |
| LNP #9 | 30 | 2 | 300 | No |
| LNP #10 | 30 | 2 | 300 | No |
| LNP #11 | 30 | 2 | 300 | Yes |

Briefly, Fab-PEG micelle post-insertion was performed by mixing preformed LNPs with Fab-PEG micelles under the following conditions:
20° C., 0.5 h, 0 rpm (no agitation)
20° C., 0.5 h, 600 rpm
20° C., 3.5 h, 0 rpm
20° C., 3.5 h, 600 rpm
40° C., 0.5 h, 0 rpm
40° C., 0.5 h, 600 rpm
40° C., 3.5 h, 0 rpm
40° C., 3.5 h, 600 rpm
30° C., 2 h, 300 rpm (two independent replicates)
30° C., 2 h, 300 rpm with preheating of LNPs to 30° C. prior to insertion

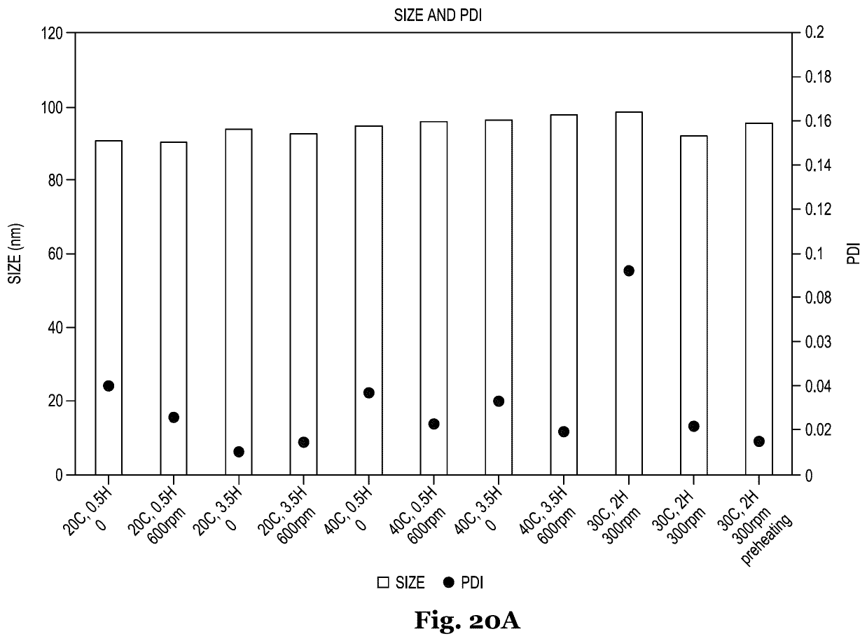

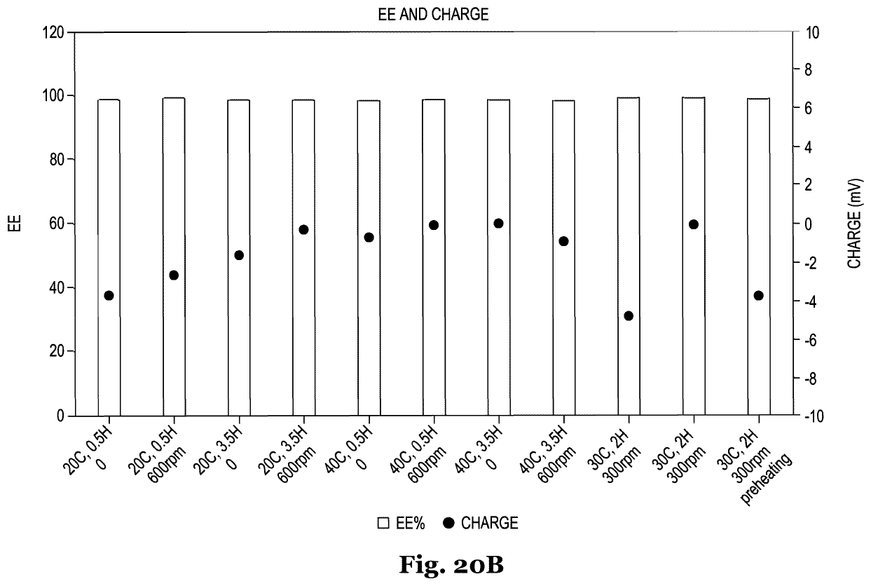

Unless otherwise specified in Table 3, no preheating was applied. After insertion, samples were equilibrated to room temperature prior to characterization. Particle size and PDI were measured with dynamic light scattering (FIG. 20A). Encapsulation efficiency (EE) for mRNA/gRNA was measured with a standard nuclease protection/quantification assays and the surface charge was calculated from the zeta potential (FIG. 20B). Ligand presentation was quantified by the preset GD input (1096 mg Ab/g RNA). Briefly, ligand presentation was not measured directly on the nanoparticle surface; instead, it was standardized and expressed by the fixed grafting-density (GD) input used in the insertion reaction-specifically, 1,096 mg of antibody (Ab) added per gram of encapsulated RNA. Briefly, no significant changes in size, PDI, encapsulation efficiency and charge were observed among the groups.

While certain embodiments have been described, these embodiments have been presented by way of example only and are not intended to limit the scope of the present disclosures. Indeed, the novel methods, apparatuses, modules, instruments and systems described herein can be embodied in a variety of other forms; furthermore, various omissions, substitutions and changes in the form of the methods, apparatuses, modules, instruments and systems described herein can be made without departing from the spirit of the present disclosures. The accompanying claims and their equivalents are intended to cover such forms or modifications as would fall within the scope and spirit of the present disclosures.

What is claimed is:

1. A process for the insertion of a targeting moiety into a lipid nanoparticle, the process comprising the steps of:
inserting the targeting moiety into the lipid nanoparticle in a reaction that combines an amount of a targeting moiety with an amount of total lipids in a lipid nanoparticle for a time-period of at least one hour at a temperature no higher than 42° C., thereby forming an amount of targeted lipid nanoparticles; and
quenching the insertion of the targeting moiety by cooling the reaction to about 20° C. or lower.

2. The process of claim 1, whereby the process yields greater than 80% of targeted nanoparticles.

3. The process of claim 1, whereby the amount of the targeting moiety in the reaction ranges from 0.15 μg of the targeting moiety per mol of the total lipids in the lipid nanoparticle to 30 g of the targeting moiety per mol of the total lipids in the lipid nanoparticle.

4. The process of claim 3, whereby the process produces a plurality of targeted nanoparticles comprising from 0.15 μg to 25 mg, from 0.15 μg to 20 mg, from 0.15 μg to 15 mg, from 0.15 μg to 10 mg, from 0.15 μg to 5 mg, from 0.15 μg to 1 mg of the targeted moiety incorporated into its outer membrane(s).

5. The process of claim 1, whereby the inserting is conducted at a pH from about 6.2 to about 7.5.

6. The process of claim 1, whereby the inserting is conducted at a pH of about 7.4.

7. The process of claim 1, whereby the quenching of the insertion is performed by cooling the reaction to about 10° C. to about 20° C.

8. The process of claim 1, whereby the quenching of the insertion is performed by cooling the reaction to about 15° C.

9. The process of claim 1, whereby the temperature is no higher than 32° C.

10. The process of claim 1, whereby the time-period is a time-period from about one hour to about four hours at a temperature between 20° C. and 32° C.

11. The process of claim 10, whereby the time-period is a time-period from about one hour to about two hours at a temperature between 20° C. and 32° C.

12. The process of claim 1, whereby the time-period is a time-period from about one hour to about four hours at a temperature between 20° C. and 42° C.

13. The process of claim 12, whereby the time-period is a time-period from about one hour to about two hours at a temperature between 20° C. and 42° C.

14. The process of claim 1, whereby the time-period is a time-period of about two hours at a temperature of about 30° C.

15. The process of claim 14, whereby the time-period is no more than four hours.

16. The process of claim 1, whereby the insertion is performed on a 2-(N-morpholino)ethanesulfonic acid (MES) buffer or a tris(hydroxymethyl)aminomethane (Tris) or a HEPES (4-(2-hydroxyethyl)-1-piperazineethanesulfonic acid) (HEPES) buffer.

17. The process of claim 16, whereby the buffer is a 25 mM MES buffer comprising 150 mM NaCl at pH 6.5, 50 mM Tris buffer comprising 150 mM NaCl at pH 7.5, or 25 mM HEPES buffer comprising 150 mM NaCl at pH 7.4.

18. The process of claim 1, wherein the targeting moiety is from about 10 kDA to about 160 kDA.

19. The process of claim 1, wherein the targeting moiety is an antigen-binding protein.

20. The process of claim 1, wherein the targeting moiety is a fragment antigen-binding (Fab).

21. The process of claim 20, whereby the amount of the Fab targeting moiety in the reaction whereby the ranges from 1 g per mol to about 10 g per mol of the Fab targeting moiety per 1 mol of the total lipids in the lipid nanoparticle.

22. The process of claim 1, wherein the targeting moiety is a variable heavy domain of a heavy chain (VHH).

23. The process of claim 22, whereby the amount of the VHH targeting moiety in the reaction whereby the ranges from about 1 g per mol to about 5 g per mol of the VHH targeting moiety per 1 mol of the total lipids in the lipid nanoparticle.

24. The process of claim 1, wherein the lipid nanoparticle comprises at least one ionizable lipid.

25. The process of claim 24, wherein the ionizable lipid is selected from the group consisting of:

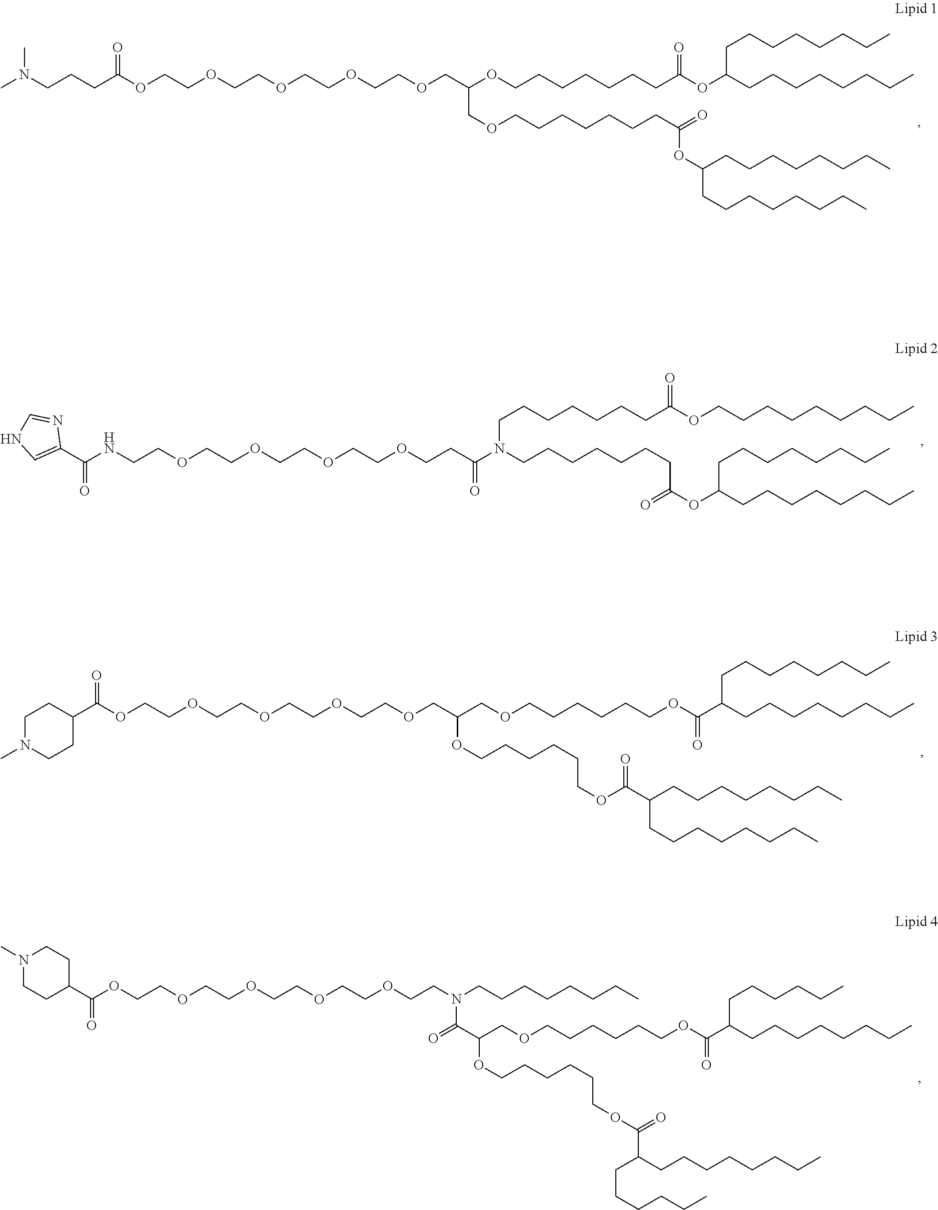

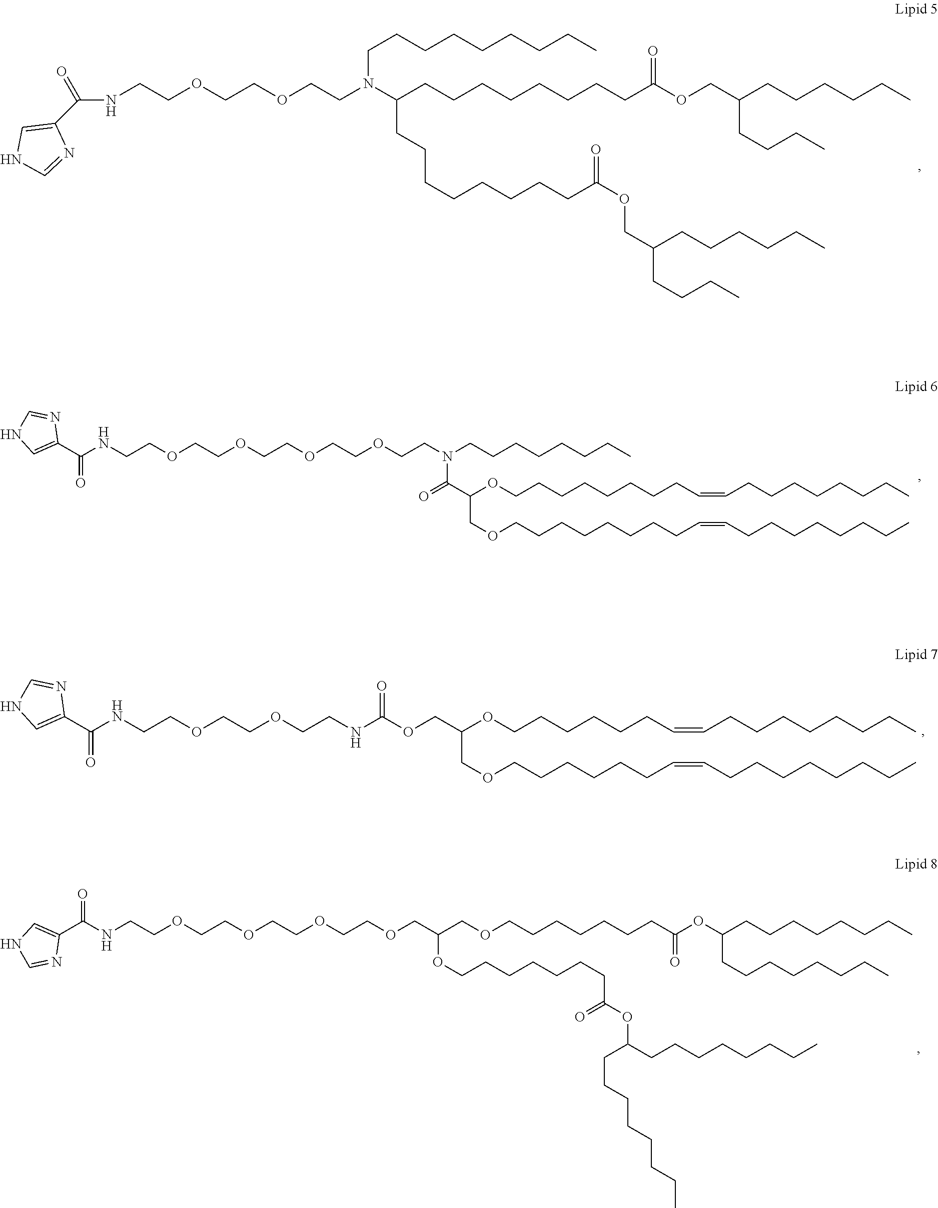 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC ; CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1 ; CCCCCCCC/C=C\CCCCCCOCC(COC(=O)NCCOCCOCCNC(=O)c1c[nH]cn1)OCCCCCC/C=C\CCCCCCCC ; CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

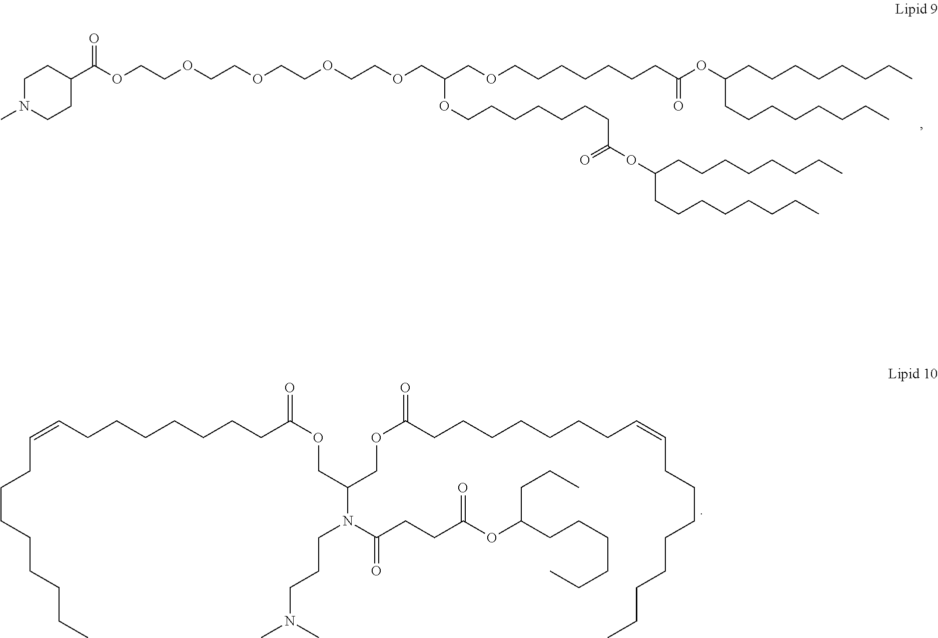 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC ; CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

26. The process of claim 1, whereby the lipid nanoparticle further comprises a nucleic acid cargo.

27. The process of claim 26, whereby the nucleic acid cargo is pre-encapsulated into the lipid nanoparticle prior to the insertion of the targeting moiety.

28. The process of claim 26, whereby insertion of the targeting moiety does not fragment the nucleic acid cargo.

29. The process of claim 26, whereby the amount of the targeting moiety is from 0.5× to 1.5× the amount of the nucleic acid cargo.

30. The process of claim 26, whereby the amount of the targeting moiety is from 800 mg to 1,200 mg of targeting moiety per gram of the nucleic acid cargo.

31. The process of claim 26, whereby greater than 50% of the nucleic acid cargo remains encapsulated in the targeted lipid nanoparticle during the reaction.
